SEQUENCE LISTING (105 sequences)

SEQ ID NO: 1 - protein, 783 aa, Homo sapiens
MSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 2 - DNA, 2352 bp, Artificial Sequence
  cDNA
atgagtggtggccaccagctgcagctggctgccctctggccctggctgctgatggctaccctgcaggcaggctttggacgcacaggactggtactggcagcagcggtggagtctgaaagatcagcagaacagaaagctattatcagagtgatccccttgaaaatggaccccacaggaaaactgaatctcactttggaaggtgtgtttgctggtgttgctgaaataactccagcagaaggaaaattaatgcagtcccacccgctgtacctgtgcaatgccagtgatgacgacaatctggagcctggattcatcagcatcgtcaagctggagagtcctcgacgggccccccgcccctgcctgtcactggctagcaaggctcggatggcgggtgagcgaggagccagtgctgtcctctttgacatcactgaggatcgagctgctgctgagcagctgcagcagccgctggggctgacctggccagtggtgttgatctggggtaatgacgctgagaagctgatggagtttgtgtacaagaaccaaaaggcccatgtgaggattgagctgaaggagcccccggcctggccagattatgatgtgtggatcctaatgacagtggtgggcaccatctttgtgatcatcctggcttcggtgctgcgcatccggtgccgcccccgccacagcaggccggatccgcttcagcagagaacagcctgggccatcagccagctggccaccaggaggtaccaggccagctgcaggcaggcccggggtgagtggccagactcagggagcagctgcagctcagcccctgtgtgtgccatctgtctggaggagttctctgaggggcaggagctacgggtcatttcctgcctccatgagttccatcgtaactgtgtggacccctggttacatcagcatcggacttgccccctctgcatgttcaacatcacagagggagattcattttcccagtccctgggaccctctcgatcttaccaagaaccaggtcgaagactccacctcattcgccagcatcccggccatgcccactaccacctccctgctgcctacctgttgggcccttcccggagtgcagtggctcggcccccacgacctggtcccttcctgccatcccaggagccaggcatgggccctcggcatcaccgcttccccagagctgcacatccccgggctccaggagagcagcagcgcctggcaggagcccagcacccctatgcacaaggctggggactgagccacctccaatccacctcacagcaccctgctgcttgcccagtgcccctacgccgggccaggccccctgacagcagtggatctggagaaagctattgcacagaacgcagtgggtacctggcagatgggccagccagtgactccagctcagggccctgtcatggctcttccagtgactctgtggtcaactgcacggacatcagcctacagggggtccatggcagcagttctactttctgcagctccctaagcagtgactttgaccccctagtgtactgcagccctaaaggggatccccagcgagtggacatgcagcctagtgtgacctctcggcctcgttccttggactcggtggtgcccacaggggaaacccaggtttccagccatgtccactaccaccgccaccggcaccaccactacaaaaagcggttccagtggcatggcaggaagcctggcccagaaaccggagtcccccagtccaggcctcctattcctcggacacagccccagccagagccaccttctcctgatcagcaagtcaccagatccaactcagcagccccttcggggcggctctctaacccacagtgccccagggccctccctgagccagcccctggcccagttgacgcctccagcatctgccccagtaccagcagtctgttcaacttgcaaaaatccagcctctctgcccgacacccacagaggaaaaggcgggggggtccctccgagcccacccctggctctcggccccaggatgcaactgtgcacccagcttgccagatttttccccattacacccccagtgtggcatatccttggtccccagaggcacaccccttgatctgtggacctccaggcctggacaagaggctgctaccagaaaccccaggcccctgttactcaaattcacagccagtgtggttgtgcctgactcctcgccagcccctggaaccacatccacctggggaggggccttctgaatggagttctgacaccgcagagggcaggccatgcccttatccgcactgccaggtgctgtcggcccagcctggctcagaggaggaactcgaggagctgtgtgaacaggctgtgtga

SEQ ID NO: 3 - protein, 936 aa, Homo sapiens
MRPRSGGRPGATGRRRRRLRRRPRGLRCSRLPPPPPLPLLLGLLLAAAGPGAARAKETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDMGIFLAFFVVVSLVCLILLVKIKLKQRRSQNSMNRLAVQALEKMETRKFNSKSKGRREGSCGALDTLSSSSTSDCAICLEKYIDGEELRVIPCTHRFHRKCVDPWLLQHHTCPHCRHNIIEQKGNPSAVCVETSNLSRGRQQRVTLPVHYPGRVHRTNAIPAYPTRTSMDSHGNPVTLLTMDRHGEQSLYSPQTPAYIRSYPPLHLDHSLAAHRCGLEHRAYSPAHPFRRPKLSGRSFSKAACFSQYETMYQHYYFQGLSYPEQEGQSPPSLAPRGPARAFPPSGSGSLLFPTVVHVAPPSHLESGSTSSFSCYHGHRSVCSGYLADCPGSDSSSSSSSGQCHCSSSDSVVDCTEVSNQGVYGSCSTFRSSLSSDYDPFIYRSRSPCRASEAGGSGSSGRGPALCFEGSPPPEELPAVHSHGAGRGEPWPGPASPSGDQVSTCSLEMNYSSNSSLEHRGPNSSTSEVGLEASPGAAPDLRRTWKGGHELPSCACCCEPQPSPAGPSAGAAGSSTLFLGPHLYEGSGPAGGEPQSGSSQGLYGLHPDHLPRTDGVKYEGLPCCFYEEKQVARGGGGGSGCYTEDYSVSVQYTLTEEPPPGCYPGARDLSQRIPIIPEDVDCDLGLPSDCQGTHSLGSWGGTRGPDTPRPHRGLGATREEERALCCQARALLRPGCPPEEAGAVRANFPSALQDTQESSTTATEAAGPRSHSADSSSPGA

SEQ ID NO: 4 - DNA, 2811 bp, Artificial Sequence
  cdna
atgaggccgcgctcgggcgggcgcccaggggccacgggccgccgccgccgccgcctgcgccgccgcccccgcggcctccggtgcagccgcctgccgccgccgccgccgctgccgctgctgctcgggctgctgctggcggccgcggggcccggcgcggcgcgggccaaggagacggcgttcgtggaggtggtgctgttcgagtcgagcccaagcggcgattacaccacctacaccaccggcctcacgggccgcttctcgcgggccggggccacgctcagcgccgagggcgagatcgtgcagatgcacccactgggcctatgtaataacaatgacgaagaggacttgtatgaatatggctgggtaggagtggtgaagctggaacagccagaattggacccgaaaccatgcctcactgtcctaggcaaggccaagcgagcagtacagcggggagctactgcagtcatctttgatgtgtctgaaaacccagaagctattgatcagctgaaccagggctctgaagacccgctcaagaggccggtggtgtatgtgaagggtgcagatgccattaagctgatgaacatcgtcaacaagcagaaagtggctcgagcaaggatccagcaccgccctcctcgacaacccactgaatactttgacatggggattttcctggctttcttcgtcgtggtctccttggtctgcctcatcctccttgtcaaaatcaagctgaagcagcgacgcagtcagaattccatgaacaggctggctgtgcaggctctagagaagatggaaaccagaaagttcaactccaagagcaaggggcgccgggaggggagctgtggggccctggacacactcagcagcagctccacgtccgactgtgccatctgtctggagaagtacattgatggagaggagctgcgggtcatcccctgtactcaccggtttcacaggaagtgcgtggacccctggctgctgcagcaccacacctgcccccactgtcggcacaacatcatagaacaaaagggaaacccaagcgcggtgtgtgtggagaccagcaacctctcacgtggtcggcagcagagggtgaccctgccggtgcattaccccggccgcgtgcacaggaccaacgccatcccagcctaccctacgaggacaagcatggactcccacggcaaccccgtcaccttgctgaccatggaccggcacggggagcagagcctctattccccgcagacccccgcctacatccgcagctacccacccctccacctggaccacagcctggccgctcaccgctgcggcctggagcaccgggcctactccccagcccaccccttccgcaggcccaagttgagtggccgcagcttctccaaggcagcttgcttctcccagtatgagaccatgtaccagcactactacttccagggcctcagctacccggagcaggaggggcagtccccacctagcctcgcaccccggggcccggcccgtgcctttcctccgagcggcagtggcagcctgctcttccccaccgtggtgcacgtggccccgccctcccacctggagagcggcagcacgtccagcttcagctgctatcacggccaccgctcggtgtgcagtggctacctggccgactgcccaggcagcgacagcagcagcagcagcagctccggccagtgccactgttcctccagtgactctgtggtagactgcactgaggtcagcaaccagggcgtgtacgggagctgctccaccttccgcagctccctcagcagcgactatgaccccttcatctaccgcagccggagcccctgtcgtgccagtgaggcggggggctcgggcagctcgggccggggacctgccctgtgcttcgagggctccccgcctcccgaggagctcccggcggtgcacagtcatggtgctgggcggggcgagccttggccgggccctgcctctccctcgggggatcaggtgtccacctgcagcctggagatgaactacagcagcaactcctccctggagcacagggggcccaatagctctacctcagaagtggggctcgaggcttctcctggggccgcccctgacctcaggaggacctggaaggggggccacgagttgccgtcgtgtgcctgctgctgcgagccccagccctccccagccgggcctagcgccggagcagctggcagcagcaccttgttcctggggccccacctctacgagggctctggcccggcgggtggggagccccagtcaggaagctcccagggcttgtacggccttcaccccgaccatttgcccaggacagatggggtgaaatacgagggtctgccctgctgcttctatgaagagaagcaggtggcccgcgggggcggagggggcagcggctgctacactgaggactactcggtgagtgtgcagtacacgctcaccgaggaaccaccgcccggctgctaccccggggcccgggacctgagccagcgcatccccatcattccagaggatgtggactgtgatctgggcctgccctcggactgccaagggacccacagcctcggctcctggggtgggacgcgaggcccggataccccacggccccacaggggcctgggagcaacccgggaagaggagcgggctctgtgctgccaggctagggccctactgcggcctggctgccctccggaggaggcgggtgctgtcagggccaacttccctagtgccctccaggacactcaggagtccagcaccactgccactgaggctgcaggaccgagatctcactcagcagacagcagcagcccgggagcctga

SEQ ID NO: 5 - protein, 381 aa, Homo sapiens
MLLSIGMLMLSATQVYTILTVQLFAFLNLLPVEADILAYNFENASQTFDDLPARFGYRLPAEGLKGFLINSKPENACEPIVPPPVKDNSSGTFIVLIRRLDCNFDIKVLNAQRAGYKAAIVHNVDSDDLISMGSNDIEVLKKIDIPSVFIGESSANSLKDEFTYEKGGHLILVPEFSLPLEYYLIPFLIIVGICLILIVIFMITKFVQDRHRARRNRLRKDQLKKLPVHKFKKGDEYDVCAICLDEYEDGDKLRILPCSHAYHCKCVDPWLTKTKKTCPVCKQKVVPSQGDSDSDTDSSQEENEVTEHTPLLRPLASVSAQSFGALSESRSHQNMTESSDYEEDDNEDTDSSDAENEINEHDVVVQLQPNGERDYNIANTV

SEQ ID NO: 6 - DNA, 1146 bp, Artificial Sequence
  cDNA
atgctgctctccatagggatgctcatgctgtcagccacacaagtctacaccatcttgactgtccagctctttgcattcttaaacctactgcctgtagaagcagacattttagcatataactttgaaaatgcatctcagacatttgatgacctccctgcaagatttggttatagacttccagctgaaggtttaaagggttttttgattaactcaaaaccagagaatgcctgtgaacccatagtgcctccaccagtaaaagacaattcatctggcactttcatcgtgttaattagaagacttgattgtaattttgatataaaggttttaaatgcacagagagcaggatacaaggcagccatagttcacaatgttgattctgatgacctcattagcatgggatccaacgacattgaggtactaaagaaaattgacattccatctgtctttattggtgaatcatcagctaattctctgaaagatgaattcacatatgaaaaagggggccaccttatcttagttccagaatttagtcttcctttggaatactacctaattcccttccttatcatagtgggcatctgtctcatcttgatagtcattttcatgatcacaaaatttgtccaggatagacatagagctagaagaaacagacttcgtaaagatcaacttaagaaacttcctgtacataaattcaagaaaggagatgagtatgatgtatgtgccatttgtttggatgagtatgaagatggagacaaactcagaatccttccctgttcccatgcttatcactgcaagtgtgtagacccttggctaactaaaaccaaaaaaacctgtccagtgtgcaagcaaaaagttgttccttctcaaggcgattcagactctgacacagacagtagtcaagaagaaaatgaagtgacagaacatacccctttactgagacctttagcttctgtcagtgcccagtcatttggggctttatcggaatcccgctcacatcagaacatgacagaatcttcagactatgaggaagacgacaatgaagatactgacagtagtgatgcagaaaatgaaattaatgaacatgatgtcgtggtccagttgcagcctaatggtgaacgggattacaacatagcaaatactgtttga

SEQ ID NO: 7 - protein, 643 aa, Homo sapiens [sequence omitted]

SEQ ID NO: 8 - DNA, 1932 bp, Artificial Sequence
  cDNA
atgccgctgctcttcctcgagcgcttcccctggcccagcctccgcacctacacgggcctcagcggcctggccctgctgggcaccatcatcagcgcctaccgcgcgctcagccagcccgaggccggccccggcgagccggaccagctaacggcctcgctgcagcctgagccgccggcgcccgcccggccgagcgccgggggaccccgggcccgcgatgtggcccagtacctgctctcagacagcctcttcgtgtgggttctagtaaataccgcttgctgtgttttgatgttggtggctaagctcatccagtgtattgtgtttggccctcttcgagtgagtgagagacagcatctcaaagacaaattttggaattttattttctacaagttcattttcatctttggtgtgctgaatgtccagacagtggaagaggtggtcatgtggtgcctctggtttgccggacttgtctttctgcacctgatggttcagctctgcaaggatcgatttgaatatctttccttctcgcccaccacgccgatgagcagccacggtcgagtcctgtccctgttggttgccatgctgctttcctgctgtggactggcggccgtctgctccatcaccggctacacccacggaatgcacaccttggctttcatggctgcagagtctcttcttgtgacagtgaggactgctcatgtgattttacgatacgtaattcacctctgggacctcaaccacgaagggacgtgggaaggaaaggggacgtatgtctattacacagactttgtcatggagctcactctcctgtccctggacctcatgcaccatattcacatgttgttatttggcaacatctggttatccatggccagcctggtcatctttatgcagctgcgttacctgtttcatgaggtgcaacgtcgaattcgtcggcacaagaactatctacgtgtggttggaaacatggaggccaggtttgcagttgcaactccagaggagctggctgtcaacaatgacgactgtgccatctgttgggactccatgcaggctgcgcggaaactgccctgtggacatcttttccacaactcctgtcttcgttcctggctagaacaagacacctcctgtccaacatgcagaatgtctcttaatattgccgacaataatcgtgtcagggaagaacatcaaggagagaacttggatgagaatttggttcctgtagcagcagccgaagggagacctcgcttaaaccaacacaatcacttcttccatttcgatgggtctcggattgcgagctggctgccgagtttttcggttgaagtgatgcacaccaccaacattcttggcattacgcaggccagcaactcccagctcaatgcaatggctcatcagattcaagagatgtttccccaggttccataccatctggtactgcaggacctccagctgacacgctcagttgaaataacaacagacaatattttagaaggacggattcaagtaccttttcctacacagcggtcagatagcatcagacctgcattgaacagtcctgtggaaaggccaagcagtgaccaggaagagggagaaacttctgctcagaccgagcgtgtgccactggacctcagtcctcgcctggaggagacgctggacttcggcgaggtggaagtggagcccagtgaggtggaagacttcgaggctcgtgggagccgcttctccaagtctgctgatgagagacagcgcatgctggtgcagcgtaaggacgaactcctccagcaagctcgcaaacgtttcttgaacaaaagttctgaagatgatgcggcctcagagagcttcctccccttggaaggtgcgtcctctgaccccgtgaccctgcgtcgaaggatgctggctgccgccgcggaacggaggcttcagaagcagcagacctcctag

SEQ ID NO: 9 - protein, 272 aa, Homo sapiens
MTSSHVCCNFLNMWKKSKISTMYYLNQDAKLSNLFLQASSPTTGTAPRSQSRLSVCPSTQDICRICHCEGDEESPLITPCRCTGTLRFVHQSCLHQWIKSSDTRCCELCKYDFIMETKLKPLRKWEKLQMTTSERRKIFCSVTFHVIAITCVVWSLYVLIDRTAEEIKQGNDNGVLEWPFWTKLVVVAIGFTGGLVFMYVQCKVYVQLWRRLKAYNRVIFVQNCPDTAKKLEKNFSCNVNTDIKDAVVVPVPQTGANSLPSAEGGPPEVVSV

SEQ ID NO: 10 - DNA, 819 bp, Artificial Sequence
  cDNA
atgaccagcagccacgtttgttgtaattttttaaatatgtggaaaaaaagcaagatatcaaccatgtattaccttaaccaagatgccaaattatctaacttgtttctccaggcaagcagcccaacaacagggacagctcccaggagccagtcaaggttgtctgtctgtccatccactcaggacatctgcagaatctgtcactgcgaaggggatgaagagagccccctcatcacaccctgtcgctgcactgggacactgcgctttgtccaccagtcctgcctccaccagtggataaagagctcagatacacgctgctgtgagctctgcaagtatgacttcataatggagaccaagctcaaacccctccggaagtgggagaaactacagatgaccacaagtgaaaggaggaaaatattctgctctgtcacattccacgtaatcgcaatcacctgtgtggtttggtctttgtatgtattgatagaccggacagcggaggaaatcaagcaaggcaatgacaatggtgtccttgaatggccattttggacaaaactggttgtggtagccattggcttcacaggaggtcttgtcttcatgtacgtacagtgtaaagtctatgttcagttgtggcgcaggctgaaggcctacaaccgtgtgatctttgtacaaaattgcccagacactgccaaaaaactggagaagaacttctcatgtaatgtaaacacagacatcaaagatgctgtggtagtgcctgtaccacaaacaggtgcaaattcactgccatctgcagagggtggcccccctgaagttgtatcagtctga

SEQ ID NO: 11 - protein, 704 aa, Homo sapiens
MESKPSRIPRRISVQPSSSLSARMMSGSRGSSLNDTYHSRDSSFRLDSEYQSTSASASASPFQSAWYSESEITQGARSRSQNQQRDHDSKRPKLSCTNCTTSAGRNVGNGLNTLSDSSWRHSQVPRSSSMVLGSFGTDLMRERRDLERRTDSSISNLMDYSHRSGDFTTSSYVQDRVPSYSQGARPKENSMSTLQLNTSSTNHQLPSEHQTILSSRDSRNSLRSNFSSRESESSRSNTQPGFSYSSSRDEAPIISNSERVVSSQRPFQESSDNEGRRTTRRLLSRIASSMSSTFFSRRSSQDSLNTRSLNSENSYVSPRILTASQSRSNVPSASEVPDNRASEASQGFRFLRRRWGLSSLSHNHSSESDSENFNQESEGRNTGPWLSSSLRNRCTPLFSRRRREGRDESSRIPTSDTSSRSHIFRRESNEVVHLEAQNDPLGAAANRPQASAASSSATTGGSTSDSAQGGRNTGISGILPGSLFRFAVPPALGSNLTDNVMITVDIIPSGWNSADGKSDKTKSAPSRDPERLQKIKESLLLEDSEEEEGDLCRICQMAAASSSNLLIEPCKCTGSLQYVHQDCMKKWLQAKINSGSSLEAVTTCELCKEKLELNLEDFDIHELHRAHANEQAEYEFISSGLYLVVLLHLCEQSFSDMMGNTNEPSTRVRFINLARTLQAHMEDLETSEDDSEEDGDHNRTFDIA

SEQ ID NO: 12 - DNA, 2115 bp, Artificial Sequence
  cDNA
atggagtctaaaccttcaaggattccaagaagaatttctgttcaaccttccagctccttaagtgctaggatgatgtctggaagcagaggaagtagtttaaatgatacctatcactcaagagactcttcatttagattggattctgaatatcagtctacatcagcatcagcatctgcgtcaccatttcaatctgcatggtatagtgaatctgagataactcagggagcacgctcaagatcgcagaaccagcaacgggatcatgattcaaaaagacctaaactttcctgtacaaactgtactacctcagctgggagaaatgttggaaatggtttaaacacattatcagattcatcttggaggcatagtcaagttcctagatcttcatcaatggtacttggatcatttggaacagacttaatgagagagaggagagatttggagagaagaacagattcctctattagtaatcttatggattatagtcaccgaagtggtgatttcacaacttcatcatatgttcaagacagagttccttcatattcacaaggagcaagaccaaaagaaaactcaatgagcactttacagttgaatacatcatccacaaaccaccaattgccttctgaacatcagaccatactaagttctagggactccagaaattctttaagatcaaatttttcttcaagagaatcagaatcttcccgaagcaatacgcagcctggattttcttacagttcaagtagagatgaagccccaatcataagcaattcagaaagggttgtttcatctcaaagaccatttcaagaatcttctgacaatgaaggtaggcggacaacgaggagattgctgtcacgcatagcttctagcatgtcatctacttttttttcacgaagatctagtcaggattccttgaatacaagatcattgaattctgaaaattcttacgtttctccaagaatcttgacagcttcacagtcccgtagtaatgtaccatcagcttctgaagttcccgataatagggcatctgaagcttctcagggatttcgatttcttaggcgaagatggggtttgtcatctcttagccacaatcatagctctgagtcagattcagaaaattttaaccaagaatctgaaggtagaaatacaggaccatggttatcttcctcacttagaaatagatgcacacctttgttctctagaaggaggcgagagggaagagatgaatcttcaaggatacctacctctgatacatcatctagatctcatatttttagaagagaatcaaatgaagtggttcaccttgaagcacagaatgatcctcttggagctgctgccaacagaccacaagcatctgcagcatcaagcagtgccacaacaggtggctctacatcagattcggctcaaggtggaagaaatacaggaatatcagggattcttcctggttccttattccggtttgcagtccctccagcacttgggagtaatttgaccgacaatgtcatgatcacagtagatattattccttcaggttggaattcagctgatggtaaaagtgataaaactaaaagtgcgccttcaagagatccagaaagattgcagaaaataaaagagagcctccttttagaggactcagaagaagaagaaggtgacttatgtagaatttgtcaaatggcagctgcatcatcatctaatttgctgatagagccatgcaagtgcacaggaagtttgcagtatgtccaccaagactgtatgaaaaagtggttacaggccaaaattaactctggttcttcattagaagctgtaaccacctgtgaactatgtaaagagaagttggagcttaacctggaggattttgatattcatgaactacatagagctcatgcaaatgaacaagctgagtatgagtttatcagctctggtctctacctagtggtgttattgcacttgtgcgaacaaagcttttctgatatgatgggaaatacaaatgaaccaagcacacgtgtccgatttattaaccttgcaagaactcttcaggcacatatggaagatctcgaaacttcagaggatgattccgaagaagacggagaccataacaggacatttgatattgcctaa

SEQ ID NO: 13 - protein, 246 aa, Homo sapiens
MTTGDCCHLPGSLCDCSGSPAFSKVVEATGLGPPQYVAQVTSRDGRLLSTVIRALDTPSDGPFCRICHEGANGECLLSPCGCTGTLGAVHKSCLEKWLSSSNTSYCELCHTEFAVEKRPRPLTEWLKDPGPRTEKRTLCCDMVCFLFITPLAAISGWLCLRGAQDHLRLHSQLEAVGLIALTIALFTIYVLWTLVSFRYHCQLYSEWRKTNQKVRLKIREADSPEGPQHSPLAAGLLKKVAEETPV

SEQ ID NO: 14 - DNA, 741 bp, Artificial Sequence
  cDNA
atgacgacgggtgactgctgccacctccccggctccctgtgtgactgctccggcagccctgccttctccaaggtcgtggaggctacgggcctcggaccgccccagtatgtggcacaggtgacttcaagggatggccggctcctctccaccgtcatccgtgccttggacacaccgagtgatggtcctttctgccggatctgccatgagggagcgaacggggagtgcttgctgtccccgtgtggctgcaccggcacgctgggtgccgtgcataagagctgtctggagaagtggctttcctcatctaacaccagctactgcgagctgtgccacacggagtttgcagtggagaaacggcctcgacccctcacagagtggctgaaggacccggggccgcggacggagaagcggacactgtgctgcgacatggtgtgtttcctgttcatcacaccgctggccgccatctcaggctggttgtgcctgcgcggggcccaggaccacctccggctccacagccagctggaggccgtgggtctcattgccctcaccatcgccctcttcaccatctatgtcctctggacgctggtctccttccgctaccactgccagctgtactccgagtggagaaagaccaaccagaaagttcgcctgaagatccgggaggcggacagccccgagggcccccagcattctccactggcagctggactcctgaagaaggtggcagaggagacaccagtatga

SEQ ID NO: 15 - protein, 291 aa, Homo sapiens
MSMPLHQISAIPSQDAISARVYRSKTKEKEREEQNEKTLGHFMSHSSNISKAGSPPSASAPAPVSSFSRTSITPSSQDICRICHCEGDDESPLITPCHCTGSLHFVHQACLQQWIKSSDTRCCELCKYEFIMETKLKPLRKWEKLQMTSSERRKIMCSVTFHVIAITCVVWSLYVLIDRTAEEIKQGQATGILEWPFWTKLVVVAIGFTGGLLFMYVQCKVYVQLWKRLKAYNRVIYVQNCPETSKKNIFEKSPLTEPNFENKHGHGICHSDTNSSCCTEPEDTGAEIIHV

SEQ ID NO: 16 - DNA, 876 bp, Artificial Sequence
  cDNA
atgagcatgccactgcatcagatctctgccattccatcccaggatgccatctctgctagagtctacagaagtaagaccaaagaaaaggagagggaagaacagaatgagaagactttgggacatttcatgagtcattcaagcaacatttctaaggctgggagtcctccgtcagcatcagctccggctccggtgtcctccttctctcgcacttctatcacgccatccagccaggacatctgcaggatctgccactgtgaaggagatgatgagagccccctgatcaccccctgccactgcacaggaagcctccacttcgtgcaccaggcctgcctgcagcagtggatcaagagctccgacacgcgctgctgcgagctctgcaagtatgagttcatcatggagaccaagctgaagccactgagaaaatgggagaagttgcagatgacgtccagcgagcgcaggaagatcatgtgctcagtgacattccacgtcattgccatcacatgtgtggtctggtccttgtatgtgctcattgaccgtactgctgaggagatcaagcaggggcaggcaacaggaatcctagaatggcccttttggactaaattggtggttgtggccatcggcttcaccggaggacttctttttatgtatgttcagtgtaaagtgtatgtgcaattgtggaagagactcaaggcctataatagagtgatctatgttcaaaactgtccagaaacaagcaaaaagaatatttttgaaaaatctccactaacagagcccaactttgaaaataaacatggacatggaatctgtcattccgacacaaactcttcttgttgcacagagcctgaagacactggagcagaaatcattcacgtctga

SEQ ID NO: 17 - protein, 346 aa, Homo sapiens
MLKSRLRMFLNELKLLVLTGGGRPRAEPQPRGGRGGGCGWAPFAGCSTRDGDGDEEEYYGSEPRARGLAGDKEPRAGPLPPPAPPLPPPGALDALSLSSSLDSGLRTPQCRICFQGPEQGELLSPCRCDGSVRCTHQPCLIRWISERGSWSCELCYFKYQVLAISTKNPLQWQAISLTVIEKVQIAAIVLGSLFLVASISWLIWSSLSPSAKWQRQDLLFQICYGMYGFMDVVCIGLIIHEGSSVYRIFKRWQAVNQQWKVLNYDKTKDIGGDAGGGTAGKSGPRNSRTGPTSGATSRPPAAQRMRTLLPQRCGYTILHLLGQLRPPDARSSSHSGREVVMRVTTV

SEQ ID NO: 18 - DNA, 1041 bp, Artificial Sequence
  cDNA
atgctcaagtctcggctccgcatgtttctgaacgagctgaagctgctggtgctgacaggcggggggcggccccgggccgagccgcaaccccgggggggccggggaggcggctgcggctgggcgcccttcgctggctgctccacccgggacggcgacggcgacgaggaggagtactacgggtcggagccgcgggcccggggcctggccggcgacaaggagccgcgggccggacccctgccgccgcccgcgccgccgctgccgcccccgggcgcgctggacgccctgtcgctcagcagtagcctggacagcggactccgaacccctcagtgccggatctgcttccagggcccggagcagggggagctcttaagcccctgccgctgcgacggctcagtgcgctgcacgcatcagccctgcctcatccgctggatcagcgagaggggctcctggagctgtgagctctgctacttcaagtaccaggtcctggcgatcagcaccaagaacccactgcagtggcaggccatctccctgacggtcatcgagaaggtccagattgctgccatagttctgggctcgctcttcctggttgccagcatctcctggctcatctggtcctcactcagcccttcagccaagtggcaacgacaggatctgctctttcagatctgctacggcatgtatggcttcatggatgtcgtctgcataggcctcatcatccatgaaggctcctctgtctaccgcatcttcaagcgctggcaggcagtgaaccagcagtggaaggtcctaaattatgacaagaccaaggacataggaggagatgcagggggagggacggcagggaagtcaggccccaggaactcacggacgggccccacctctggggccacgagccgccccccagctgcccagcgcatgcggacgctcttgcctcagcgctgcggttatacaatcttgcacctccttgggcagctgcggccaccagatgcccgttccagctcccattctggccgagaggttgtcatgagggtcactacagtctga

SEQ ID NO: 19 - protein, 400 aa, Homo sapiens
MAWRRREASVGARGVLALALLALALCVPGARGRALEWFSAVVNIEYVDPQTNLTVWSVSESGRFGDSSPKEGAHGLVGVPWAPGGDLEGCAPDTRFFVPEPGGRGAAPWVALVARGGCTFKDKVLVAARRNASAVVLYNEERYGNITLPMSHAGTGNIVVIMISYPKGREILELVQKGIPVTMTIGVGTRHVQEFISGQSVVFVAIAFITMMIISLAWLIFYYIQRFLYTGSQIGSQSHRKETKKVIGQLLLHTVKHGEKGIDVDAENCAVCIENFKVKDIIRILPCKHIFHRICIDPWLLDHRTCPMCKLDVIKALGYWGEPGDVQEMPAPESPPGRDPAANLSLALPDDDGSDDSSPPSASPAESEPQCDPSFKGDAGENTALLEAGRSDSRHGGPIS

SEQ ID NO: 20 - DNA, 1203 bp, Artificial Sequence
  cDNA
atggcgtggcggcggcgcgaagccagcgtcggggctcgcggcgtgttggctctggcgttgctcgccctggccctgtgcgtgcccggggcccggggccgggctctcgagtggttctcggccgtggtaaacatcgagtacgtggacccgcagaccaacctgacggtgtggagcgtctcggagagtggccgcttcggcgacagctcgcccaaggagggcgcgcatggcctggtgggcgtcccgtgggcgcccggcggagacctcgagggctgcgcgcccgacacgcgcttcttcgtgcccgagcccggcggccgaggggccgcgccctgggtcgccctggtggctcgtgggggctgcaccttcaaggacaaggtgctggtggcggcgcggaggaacgcctcggccgtcgtcctctacaatgaggagcgctacgggaacatcaccttgcccatgtctcacgcgggaacaggaaatatagtggtcattatgattagctatccaaaaggaagagaaattttggagctggtgcaaaaaggaattccagtaacgatgaccataggggttggcacccggcatgtacaggagttcatcagcggtcagtctgtggtgtttgtggccattgccttcatcaccatgatgattatctcgttagcctggctaatattttactatatacagcgtttcctatatactggctctcagattggaagtcagagccatagaaaagaaactaagaaagttattggccagcttctacttcatactgtaaagcatggagaaaagggaattgatgttgatgctgaaaattgtgcagtgtgtattgaaaatttcaaagtaaaggatattattagaattctgccatgcaagcatatttttcatagaatatgcattgacccatggcttttggatcaccgaacatgtccaatgtgtaaacttgatgtcatcaaagccctaggatattggggagagcctggggatgtacaggagatgcctgctccagaatctcctcctggaagggatccagctgcaaatttgagtctagctttaccagatgatgacggaagtgatgacagcagtccaccatcagcctcccctgctgaatctgagccacagtgtgatcccagctttaaaggagatgcaggagaaaatacggcattgctagaagccggcaggagtgactctcggcatggaggacccatctcctag

SEQ ID NO: 21 - protein, 663 aa, Homo sapiens
MAAKEKLEAVLNVALRVPSIMLLDVLYRWDVSSFFQQIQRSSLSNNPLFQYKYLALNMHYVGYILSVVLLTLPRQHLVQLYLYFLTALLLYAGHQISRDYVRSELEFAYEGPMYLEPLSMNRFTTALIGQLVVCTLCSCVMKTKQIWLFSAHMLPLLARLCLVPLETIVIINKFAMIFTGLEVLYFLGSNLLVPYNLAKSAYRELVQVVEVYGLLALGMSLWNQLVVPVLFMVFWLVLFALQIYSYFSTRDQPASRERLLFLFLTSIAECCSTPYSLLGLVFTVSFVALGVLTLCKFYLQGYRAFMNDPAMNRGMTEGVTLLILAVQTGLIELQVVHRAFLLSIILFIVVASILQSMLEIADPIVLALGASRDKSLWKHFRAVSLCLFLLVFPAYMAYMICQFFHMDFWLLIIISSSILTSLQVLGTLFIYVLFMVEEFRKEPVENMDDVIYYVNGTYRLLEFLVALCVVAYGVSETIFGEWTVMGSMIIFIHSYYNVWLRAQLGWKSFLLRRDAVNKIKSLPIATKEQLEKHNDICAICYQDMKSAVITPCSHFFHAGCLKKWLYVQETCPLCHCHLKNSSQLPGLGTEPVLQPHAGAEQNVMFQEGTEPPGQEHTPGTRIQEGSRDNNEYIARRPDNQEGAFDPKEYPHSAKDEAHPVESA

SEQ ID NO: 22 - DNA, 1992 bp, Artificial Sequence
  cDNA
atggctgcaaaggagaaactggaggcagtgttaaatgtggccctgagggtgccaagcatcatgctgttggatgtcctgtacagatgggatgtcagctcctttttccagcagatccaaagaagtagccttagtaataaccctcttttccagtataagtatttggctcttaatatgcattatgtaggttatatcttaagtgtggtgctgctaacattgcccaggcagcatctggttcagctttatctatattttttgactgctctgctcctctatgctggacatcaaatttccagggactatgttcggagtgaactggagtttgcctatgagggaccaatgtatttagaacctctctctatgaatcggtttaccacagccttaataggtcagttggtggtgtgtactttatgctcctgtgtcatgaaaacaaagcagatttggctgttttcagctcacatgcttcctctgctagcacgactctgccttgttcctttggagacaattgttatcatcaataaatttgctatgatttttactggattggaagttctctattttcttgggtctaatcttttggtaccttataaccttgctaaatctgcatacagagaattggttcaggtagtggaggtatatggccttctcgccttgggaatgtccctgtggaatcaactggtagtccctgttcttttcatggttttctggctcgtcttatttgctcttcagatttactcctatttcagtactcgagatcagcctgcatcacgtgagaggcttcttttcctttttctgacaagtattgcggaatgctgcagcactccttactctcttttgggtttggtcttcacggtttcttttgttgccttgggtgttctcacactctgcaagttttacttgcagggttatcgagctttcatgaatgatcctgccatgaatcggggcatgacagaaggagtaacgctgttaatcctggcagtgcagactgggctgatagaactgcaggttgttcatcgggcattcttgctcagtattatccttttcattgtcgtagcttctatcctacagtctatgttagaaattgcagatcctattgttttggcactgggagcatctagagacaagagcttgtggaaacacttccgtgctgtaagcctttgtttatttttattggtattccctgcttatatggcttatatgatttgccagtttttccacatggatttttggcttcttatcattatttccagcagcattcttacctctcttcaggttctgggaacactttttatttatgtcttatttatggttgaggaattcagaaaagagccagtggaaaacatggatgatgtcatctactatgtgaatggcacttaccgcctgctggagtttcttgtggccctctgtgtggtggcctatggcgtctcagagaccatctttggagaatggacagtgatgggctcaatgatcatcttcattcattcctactataacgtgtggcttcgggcccagctggggtggaagagctttcttctccgcagggatgctgtgaataagattaaatcgttacccattgctacgaaagagcagcttgagaaacacaatgatatttgtgccatctgttatcaggacatgaaatctgctgtgatcacgccttgcagtcattttttccatgcaggctgtcttaagaaatggctgtatgtccaggagacctgccctctgtgccactgccatctgaaaaactcctcccagcttccaggattaggaactgagccagttctacagcctcatgctggagctgagcaaaacgtcatgtttcaggaaggtactgaacccccaggccaggagcatactccagggaccaggatacaggaaggttccagggacaataatgagtacattgccagacgaccagataaccaggaaggggcttttgaccccaaagaatatcctcacagtgcgaaagatgaagcacatcctgttgaatcagcctag

SEQ ID NO: 23 - protein, 435 aa, Homo sapiens
MPLFLLSLPTPPSASGHERRQRPEAKTSGSEKKYLRAMQANRSQLHSPPGTGSSEDASTPQCVHTRLTGEGSCPHSGDVHIQINSIPKECAENASSRNIRSGVHSCAHGCVHSRLRGHSHSEARLTDDTAAESGDHGSSSFSEFRYLFKWLQKSLPYILILSVKLVMQHITGISLGIGLLTTFMYANKSIVNQVFLRERSSKIQCAWLLVFLAGSSVLLYYTFHSQSLYYSLIFLNPTLDHLSFWEVFWIVGITDFILKFFFMGLKCLILLVPSFIMPFKSKGYWYMLLEELCQYYRTFVPIPVWFRYLISYGEFGNVTRWSLGILLALLYLILKLLEFFGHLRTFRQVLRIFFTQPSYGVAASKRQCSDVDDICSICQAEFQKPILLICQHIFCEECMTLWFNREKTCPLCRTVISDHINKWKDGATSSHLQIY

SEQ ID NO: 24 - DNA, 1308 bp, Artificial Sequence
  cDNA
atgccgctgttcttgctgtcgctcccgacacctccgtccgcttctggtcatgagaggagacagaggcctgaagcaaagacatctgggtcagagaaaaagtatttaagggccatgcaagccaatcgtagccaactgcacagtcctccaggaactggaagcagtgaggatgcctcaacccctcagtgtgtccacacaagattgacaggagagggttcttgccctcattctggagatgttcatatccagataaactccatacctaaagaatgtgcagaaaatgcaagctccagaaatataaggtcaggtgtccatagctgtgcccatggatgtgtacacagtcgcttacggggtcactcccacagtgaagcaaggctgactgatgatactgccgcagaatctggagatcatggtagtagctccttctcagaattccgctatctcttcaagtggctgcaaaaaagtcttccatatattttgattctgagcgtcaaacttgttatgcagcatataacaggaatttctcttggaattgggctgctaacaacttttatgtatgcaaacaaaagcattgtaaatcaggtttttctaagagaaaggtcctcaaagattcagtgtgcttggttactggtattcttagcaggatcttctgttcttttatattacacctttcattctcagtcactttattacagcttaatttttttaaatcctactttggaccatttgagcttctgggaagtattttggattgttggaattacagacttcattctgaaattctttttcatgggcttaaaatgccttattttattggtgccttctttcatcatgccttttaaatctaagggttactggtatatgcttttagaagaattgtgtcaatactaccgaacttttgttcccataccagtttggtttcgctaccttataagctatggggagtttggtaacgtaactagatggagtcttgggatactgctggctttactctacctcatattaaaacttttggaattttttgggcatctgagaactttcagacaggttttacgaatattttttacacaaccaagttatggagtggctgccagcaagagacagtgttcagatgtggatgatatttgttcaatatgtcaagctgaatttcagaagccaattcttctcatttgtcagcatatattttgtgaagagtgcatgaccttatggtttaacagagagaaaacatgtccactctgcagaactgtgatttcagaccatataaacaaatggaaggatggagccacttcatcacaccttcaaatatattaa

SEQ ID NO: 25 - protein, 350 aa, Homo sapiens [sequence omitted]

SEQ ID NO: 26 - DNA, 1053 bp, Artificial Sequence
  cDNA
atgcaccctgcagccttcccgcttcctgtggttgtggccgctgtgctgtggggagcggccccgacccgggggctcattcgagcgacctcggaccacaatgccagcatggactttgcagaccttccagctctgtttggggctaccttgagccaggagggcctccaggggttccttgtggaggctcacccagacaatgcctgcagccccattgccccaccacccccagccccggtcaatgggtcagtctttattgcgctgcttcgaagattcgactgcaactttgacctcaaggtcctaaatgcccagaaggctggatatggtgccgctgtagtacacaatgtgaattccaatgaacttctgaacatggtgtggaatagtgaggaaatccagcagcagatctggatcccgtctgtatttattggggagagaagctccgagtacctgcgtgccctctttgtctacgagaagggggctcgggtgcttctggttccagacaataccttccccttgggctattacctcatccctttcacagggattgtgggactgctggttttggccatgggagcagtaatgatagctcgttgtatccagcaccggaaacggctccagcggaatcgacttaccaaagagcaactgaaacagattcctacacatgactatcagaagggagaccagtatgatgtctgtgccatttgcctggatgaatatgaggatggggacaagctgcgggtactcccctgtgctcatgcctaccacagccgctgcgtggacccctggctcactcagacccggaagacctgccccatttgcaagcagcctgttcatcggggtcctggggacgaagaccaagaggaagaaactcaagggcaagaggagggtgatgaaggggagccaagggaccaccctgcctcagaaaggaccccacttttgggttctagccccactcttcccacctcctttggttccttagccccagctccccttgtttttcctgggccttcaacagatcccccactgtcccctccctcttcccctgttatcctggtctaa

SEQ ID NO: 27 - protein, 419 aa, Homo sapiens
MSCAGRAGPARLAALALLTCSLWPARADNASQEYYTALINVTVQEPGRGAPLTFRIDRGRYGLDSPKAEVRGQVLAPLPLHGVADHLGCDPQTRFFVPPNIKQWIALLQRGNCTFKEKISRAAFHNAVAVVIYNNKSKEEPVTMTHPGTGDIIAVMITELRGKDILSYLEKNISVQMTIAVGTRMPPKNFSRGSLVFVSISFIVLMIISSAWLIFYFIQKIRYTNARDRNQRRLGDAAKKAISKLTTRTVKKGDKETDPDFDHCAVCIESYKQNDVVRILPCKHVFHKSCVDPWLSEHCTCPMCKLNILKALGIVPNLPCTDNVAFDMERLTRTQAVNRRSALGDLAGDNSLGLEPLRTSGISPLPQDGELTPRTGEINIAVTKEWFIIASFGLLSALTLCYMIIRATASLNANEVEWF

SEQ ID NO: 28 - DNA, 1260 bp, Artificial Sequence
  cDNA
atgagctgcgcggggcgggcgggccctgcccggctcgccgcgctcgccctgctgacctgcagcctgtggccggcacgggcagacaacgcgagccaggagtactacacggcgctcatcaacgtgacggtgcaggagcccggccgcggcgccccgctcacgtttcgcatcgaccgcgggcgctacgggcttgactcccccaaggccgaggtccgcggccaggtgctggcgccgctgcccctccacggagttgctgatcatctgggctgtgatccacaaacccggttctttgtccctcctaatatcaaacagtggattgccttgctgcagaggggaaactgcacgtttaaagagaaaatatcacgggccgctttccacaatgcagttgctgtagtcatctacaataataaatccaaagaggagccagttaccatgactcatccaggcactggagatattattgctgtcatgataacagaattgaggggtaaggatattttgagttatctggagaaaaacatctctgtacaaatgacaatagctgttggaactcgaatgccaccgaagaacttcagccgtggctctctagtcttcgtgtcaatatcctttattgttttgatgattatttcttcagcatggctcatattctacttcattcagaagatcaggtacacaaatgcacgcgacaggaaccagcgtcgtctcggagatgcagccaagaaagccatcagtaaattgacaaccaggacagtaaagaagggtgacaaggaaactgacccagactttgatcattgtgcagtctgcatagagagctataagcagaatgatgtcgtccgaattctcccctgcaagcatgttttccacaaatcctgcgtggatccctggcttagtgaacattgtacctgtcctatgtgcaaacttaatatattgaaggccctgggaattgtgccgaatttgccatgtactgataacgtagcattcgatatggaaaggctcaccagaacccaagctgttaaccgaagatcagccctcggcgacctcgccggcgacaactcccttggccttgagccacttcgaacttcggggatctcacctcttcctcaggatggggagctcactccgagaacaggagaaatcaacattgcagtaacaaaagaatggtttattattgccagttttggcctcctcagtgccctcacactctgctacatgatcatcagagccacagctagcttgaatgctaatgaggtagaatggttttga

SEQ ID NO: 29 - protein, 428 aa, Homo sapiens
MGPPPGAGVSCRGGCGFSRLLAWCFLLALSPQAPGSRGAEAVWTAYLNVSWRVPHTGVNRTVWELSEEGVYGQDSPLEPVAGVLVPPDGPGALNACNPHTNFTVPTVWGSTVQVSWLALIQRGGGCTFADKIHLAYERGASGAVIFNFPGTRNEVIPMSHPGAVDIVAIMIGNLKGTKILQSIQRGIQVTMVIEVGKKHGPWVNHYSIFFVSVSFFIITAATVGYFIFYSARRLRNARAQSRKQRQLKADAKKAIGRLQLRTLKQGDKEIGPDGDSCAVCIELYKPNDLVRILTCNHIFHKTCVDPWLLEHRTCPMCKCDILKALGIEVDVEDGSVSLQVPVSNEISNSASSHEEDNRSETASSGYASVQGTDEPPLEEHVQSTNESLQLVNHEANSVAVDVIPHVDNPTFEEDETPNQETAVREIKS

SEQ ID NO: 30 - DNA, 1287 bp, Artificial Sequence
  cDNA
atggggccgccgcctggggccggggtctcctgccgcggtggctgcggcttttccagattgctggcatggtgcttcctgctggccctgagtccgcaggcacccggttcccggggggctgaagcagtgtggaccgcgtacctcaacgtgtcctggcgggttccgcacacgggagtgaaccgtacggtgtgggagctgagcgaggagggcgtgtacggccaggactcgccgctggagcctgtggctggggtcctggtaccgcccgacgggcccggggcgcttaacgcctgtaacccgcacacgaatttcacggtgcccacggtttggggaagcaccgtgcaagtctcttggttggccctcatccaacgcggcgggggctgcaccttcgcagacaagatccatctggcttatgagagaggggcgtctggagccgtcatctttaacttccccgggacccgcaatgaggtcatccccatgtctcacccgggtgcagtagacattgttgcaatcatgatcggcaatctgaaaggcacaaaaattctgcaatctattcaaagaggcatacaagtgacaatggtcatagaagtagggaaaaaacatggcccttgggtgaatcactattcaatttttttcgtttctgtgtccttttttattattacggcggcaactgtgggctattttatcttttattctgctcgaaggctacggaatgcaagagctcaaagcaggaagcagaggcaattaaaggcagatgctaaaaaagctattggaaggcttcaactacgcacactgaaacaaggagacaaggaaattggccctgatggagatagttgtgctgtgtgcattgaattgtataaaccaaatgatttggtacgcatcttaacgtgcaaccatattttccataagacatgtgttgacccatggctgttagaacacaggacttgccccatgtgcaaatgtgacatactcaaagctttgggaattgaggtggatgttgaagatggatcagtgtctttacaagtccctgtatccaatgaaatatctaatagtgcctcctcccatgaagaggataatcgcagcgagaccgcatcatctggatatgcttcagtacagggaacagatgaaccgcctctggaggaacacgtgcagtcaacaaatgaaagtctacagctggtaaaccatgaagcaaattctgtggcagtggatgttattcctcatgttgacaacccaacctttgaagaagacgaaactcctaatcaagagactgctgttcgagaaattaaatcttaa

SEQ ID NO: 31 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 32 - DNA, 1512 bp, Artificial Sequence
  cDNA
atggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaaccgcactgtcattcaccatcgagtgccaaatgaagaggacccttcattagatcgcccttttatttcagagggtactacgttgaaagacttaatttatgatatgacaacgtcaggttctggctcaggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatgtaa

SEQ ID NO: 33 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 34 - DNA, 1779 bp, Artificial Sequence
  cDNA
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatag

SEQ ID NO: 35 - protein, 1210 aa, Homo sapiens [sequence omitted]

SEQ ID NO: 36 - DNA, 3633 bp, Artificial Sequence
  cDNA
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 37 - protein, 1255 aa, Homo sapiens [sequence omitted]

SEQ ID NO: 38 - DNA, 3768 bp, Artificial Sequence
  cDNA
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtga

SEQ ID NO: 39 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 40 - DNA, 4029 bp, Artificial Sequence
  cDNA
atgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaa

SEQ ID NO: 41 - protein, 355 aa, Homo sapiens
MGPGRCLLTALLLLALAPPPEASQYCGRLEYWNPDNKCCSSCLQRFGPPPCPDYEFRENCGLNDHGDFVTPPFRKCSSGQCNPDGAELCSPCGGGAVTPTPAAGGGRTPWRCRERPVPAKGHCPLTPGNPGAPSSQERSSPASSIAWRTPEPVPQQAWPNFLPLVVLVLLLTLAVIAILLFILLWHLCWPKEKADPYPYPGLVCGVPNTHTPSSSHLSSPGALETGDTWKEASLLPLLSRELSSLASQPLSRLLDELEVLEELIVLLDPEPGPGGGMAHGTTRHLAARYGLPAAWSTFAYSLRPSRSPLRALIEMVVAREPSASLGQLGTHLAQLGRADALRVLSKLGSSGVCWA

SEQ ID NO: 42 - DNA, 1068 bp, Artificial Sequence
  cDNA
atggggcctggacgatgcctcctgacggccttgttgcttctggccctggcgccaccgccggaagcctcccagtactgcggccgccttgaatactggaacccagacaacaagtgctgcagcagctgcctgcaacgcttcgggccgcccccctgcccggactatgagttccgggaaaactgcggactcaatgaccacggcgatttcgtaacgcccccgttccgaaagtgttcttctgggcagtgcaaccccgacggcgcggagctatgtagcccctgcggcggcggagccgtgacccctactcccgccgcgggcgggggcagaaccccgtggcgctgcagagagaggccggtccctgccaaggggcactgccccctcacacctggaaacccaggcgcccctagctcccaggagcgcagctcaccagcaagttccattgcctggaggacccctgagcctgtccctcagcaggcctggccgaatttccttccgctcgtggtgctggtcctgctcctgaccttggcggtgatagcgatcctcctgtttattctgctctggcatctctgctggcccaaggagaaagccgacccctatccctatcctggcttggtctgcggagtccccaacacccacaccccttcctcctcgcatctgtcctccccaggcgccctggagacaggggacacatggaaggaggcctcactacttccactcctgagcagggaactgtccagtctggcgtcacaacccctgtctcgcctcctggatgagctggaggtgctggaagagctgattgtactgctggaccctgagcctgggccaggtgggggtatggcccatggcactactcgacacctggccgcaagatatgggctgcctgctgcctggtccacctttgcctattcgctgaggccgagtcgctcgccgctgcgggctctgattgagatggtggtggcaagggagccctctgcctccctgggccagcttggcacacacctcgcccagctagggcgggcagatgcattgcgggtgctgtccaagcttggctcatctggggtttgctgggcttaa

SEQ ID NO: 43 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 44 - DNA, 4227 bp, Artificial Sequence
  cDNA
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtacttggtggaaagaacctctcaacattgtcagttttctattttgctttgccagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatag

SEQ ID NO: 45 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 46 - DNA, 4071 bp, Artificial Sequence
  cDNA
atgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaa

SEQ ID NO: 47 - protein, 976 aa, Homo sapiens [sequence omitted]

SEQ ID NO: 48 - DNA, 2919 bp, artificial sequence
  cDNA
atgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctga

SEQ ID NO: 49 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 50 - DNA, 2982 bp, Artificial Sequence
  cDNA
atgccggcgttggcgcgcgacggcggccagctgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaagctgccgctgtggaagtggatgtatctgcttccatcacactgcaagtgctggtcgacgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggacggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtagaaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtag

SEQ ID NO: 51 - protein, 643 aa, Homo sapiens [sequence omitted]

SEQ ID NO: 52 - DNA, 1932 bp, Artificial Sequence; identical to SEQ ID NO: 8
  cDNA

SEQ ID NO: 53 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 54 - DNA, 3270 bp, Artificial Sequence
  cDNA
atggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggatgacatcggcatagactcttcagacctggtggaagacagcttcctgtaa

SEQ ID NO: 55 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 56 - DNA, 3321 bp, Artificial Sequence
  cDNA
atgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtag

SEQ ID NO: 57 - protein, 647 aa, Homo sapiens
MAEEEAPKKSRAAGGGASWELCAGALSARLAEEGSGDAGGRRRPPVDPRRLARQLLLLLWLLEAPLLLGVRAQAAGQGPGQGPGPGQQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNPQHGGGGHRGGFPGGAGASERGKFSCPRALKVPSYLNYHFLGEKDCGAPCEPTKVYGLMYFGPEELRFSRTWIGIWSVLCCASTLFTVLTYLVDMRRFSYPERPIIFLSGCYTAVAVAYIAGFLLEDRVVCNDKFAEDGARTVAQGTKKEGCTILFMMLYFFSMASSIWWVILSLTWFLAAGMKWGHEAIEANSQYFHLAAWAVPAIKTITILALGQVDGDVLSGVCFVGLNNVDALRGFVLAPLFVYLFIGTSFLLAGFVSLFRIRTIMKHDGTKTEKLEKLMVRIGVFSVLYTVPATIVIACYFYEQAFRDQWERSWVAQSCKSYAIPCPHLQAGGGAPPHPPMSPDFTVFMIKYLMTLIVGITSGFWIWSGKTLNSWRKFYTRLTNSKQGETTV

SEQ ID NO: 58 - DNA, 1944 bp, Artificial Sequence
  cDNA
atggctgaggaggaggcgcctaagaagtcccgggccgccggcggtggcgcgagctgggaactttgtgccggggcgctctcggcccggctggcggaggagggcagcggggacgccggtggccgccgccgcccgccagttgacccccggcgattggcgcgccagctgctgctgctgctttggctgctggaggctccgctgctgctgggggtccgggcccaggcggcgggccaggggccaggccaggggcccgggccggggcagcaaccgccgccgccgcctcagcagcaacagagcgggcagcagtacaacggcgagcggggcatctccgtcccggaccacggctattgccagcccatctccatcccgctgtgcacggacatcgcgtacaaccagaccatcatgcccaacctgctgggccacacgaaccaggaggacgcgggcctggaggtgcaccagttctaccctctagtgaaagtgcagtgttccgctgagctcaagttcttcctgtgctccatgtacgcgcccgtgtgcaccgtgctagagcaggcgctgccgccctgccgctccctgtgcgagcgcgcgcgccagggctgcgaggcgctcatgaacaagttcggcttccagtggccagacacgctcaagtgtgagaagttcccggtgcacggcgccggcgagctgtgcgtgggccagaacacgtccgacaagggcaccccgacgccctcgctgcttccagagttctggaccagcaaccctcagcacggcggcggagggcaccgtggcggcttcccggggggcgccggcgcgtcggagcgaggcaagttctcctgcccgcgcgccctcaaggtgccctcctacctcaactaccacttcctgggggagaaggactgcggcgcaccttgtgagccgaccaaggtgtatgggctcatgtacttcgggcccgaggagctgcgcttctcgcgcacctggattggcatttggtcagtgctgtgctgcgcctccacgctcttcacggtgcttacgtacctggtggacatgcggcgcttcagctacccggagcggcccatcatcttcttgtccggctgttacacggccgtggccgtggcctacatcgccggcttcctcctggaagaccgagtggtgtgtaatgacaagttcgccgaggacggggcacgcactgtggcgcagggcaccaagaaggagggctgcaccatcctcttcatgatgctctacttcttcagcatggccagctccatctggtgggtgatcctgtcgctcacctggttcctggcggctggcatgaagtggggccacgaggccatcgaagccaactcacagtattttcacctggccgcctgggctgtgccggccatcaagaccatcaccatcctggcgctgggccaggtggacggcgatgtgctgagcggagtgtgcttcgtggggcttaacaacgtggacgcgctgcgtggcttcgtgctggcgcccctcttcgtgtacctgtttatcggcacgtcctttctgctggccggctttgtgtcgctcttccgcatccgcaccatcatgaagcacgatggcaccaagaccgagaagctggagaagctcatggtgcgcattggcgtcttcagcgtgctgtacactgtgccagccaccatcgtcatcgcctgctacttctacgagcaggccttccgggaccagtgggaacgcagctgggtggcccagagctgcaagagctacgctatcccctgccctcacctccaggcgggcggaggcgccccgccgcacccgcccatgagcccggacttcacggtcttcatgattaagtaccttatgacgctgatcgtgggcatcacgtcgggcttctggatctggtccggcaagaccctcaactcctggaggaagttctacacgaggctcaccaacagcaaacaaggggagactacagtctga

SEQ ID NO: 59 - protein, 565 aa, Homo sapiens
MRPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPPGLQPGAGGTPGGPGGGGAPPRYATLEHPFHCPRVLKVPSYLSYKFLGERDCAAPCEPARPDGSMFFSQEETRFARLWILTWSVLCCASTFFTVTTYLVDMQRFRYPERPIIFLSGCYTMVSVAYIAGFVLQERVVCNERFSEDGYRTVVQGTKKEGCTILFMMLYFFSMASSIWWVILSLTWFLAAGMKWGHEAIEANSQYFHLAAWAVPAVKTITILAMGQIDGDLLSGVCFVGLNSLDPLRGFVLAPLFVYLFIGTSFLLAGFVSLFRIRTIMKHDGTKTEKLERLMVRIGVFSVLYTVPATIVIACYFYEQAFREHWERSWVSQHCKSLAIPCPAHYTPRMSPDFTVYMIKYLMTLIVGITSGFWIWSGKTLHSWRKFYTRLTNSRHGETTV

SEQ ID NO: 60 - DNA, 1698 bp, Artificial Sequence
  cDNA
atgcggccccgcagcgccctgccccgcctgctgctgccgctgctgctgctgcccgccgccgggccggcccagttccacggggagaagggcatctccatcccggaccacggcttctgccagcccatctccatcccgctgtgcacggacatcgcctacaaccagaccatcatgcccaaccttctgggccacacgaaccaggaggacgcaggcctagaggtgcaccagttctatccgctggtgaaggtgcagtgctcgcccgaactgcgcttcttcctgtgctccatgtacgcacccgtgtgcaccgtgctggaacaggccatcccgccgtgccgctctatctgtgagcgcgcgcgccagggctgcgaagccctcatgaacaagttcggttttcagtggcccgagcgcctgcgctgcgagcacttcccgcgccacggcgccgagcagatctgcgtcggccagaaccactccgaggacggagctcccgcgctactcaccaccgcgccgccgccgggactgcagccgggtgccgggggcaccccgggtggcccgggcggcggcggcgctcccccgcgctacgccacgctggagcaccccttccactgcccgcgcgtcctcaaggtgccatcctatctcagctacaagtttctgggcgagcgtgattgtgctgcgccctgcgaacctgcgcggcccgatggttccatgttcttctcacaggaggagacgcgtttcgcgcgcctctggatcctcacctggtcggtgctgtgctgcgcttccaccttcttcactgtcaccacgtacttggtagacatgcagcgcttccgctacccagagcggcctatcatttttctgtcgggctgctacaccatggtgtcggtggcctacatcgcgggcttcgtgctccaggagcgcgtggtgtgcaacgagcgcttctccgaggacggttaccgcacggtggtgcagggcaccaagaaggagggctgcaccatcctcttcatgatgctctacttcttcagcatggccagctccatctggtgggtcatcctgtcgctcacctggttcctggcagccggcatgaagtggggccacgaggccatcgaggccaactctcagtacttccacctggccgcctgggccgtgccggccgtcaagaccatcaccatcctggccatgggccagatcgacggcgacctgctgagcggcgtgtgcttcgtaggcctcaacagcctggacccgctgcggggcttcgtgctagcgccgctcttcgtgtacctgttcatcggcacgtccttcctcctggccggcttcgtgtcgctcttccgcatccgcaccatcatgaagcacgacggcaccaagaccgaaaagctggagcggctcatggtgcgcatcggcgtcttctccgtgctctacacagtgcccgccaccatcgtcatcgcttgctacttctacgagcaggccttccgcgagcactgggagcgctcgtgggtgagccagcactgcaagagcctggccatcccgtgcccggcgcactacacgccgcgcatgtcgcccgacttcacggtctacatgatcaaatacctcatgacgctcatcgtgggcatcacgtcgggcttctggatctggtcgggcaagacgctgcactcgtggaggaagttctacactcgcctcaccaacagccgacacggtgagaccaccgtgtga

SEQ ID NO: 61 - protein, 666 aa, Homo sapiens
MAMTWIVFSLWPLTVFMGHIGGHSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDLNLAGEPTEGAPVAVQRDYGFWCPRELKIDPDLGYSFLHVRDCSPPCPNMYFRREELSFARYFIGLISIICLSATLFTFLTFLIDVTRFRYPERPIIFYAVCYMMVSLIFFIGFLLEDRVACNASIPAQYKASTVTQGSHNKACTMLFMILYFFTMAGSVWWVILTITWFLAAVPKWGSEAIEKKALLFHASAWGIPGTLTIILLAMNKIEGDNISGVCFVGLYDVDALRYFVLAPLCLYVVVGVSLLLAGIISLNRVRIEIPLEKENQDKLVKFMIRIGVFSILYLVPLLVVIGCYFYEQAYRGIWETTWIQERCREYHIPCPYQVTQMSRPDLILFLMKYLMALIVGIPSVFWVGSKKTCFEWASFFHGRRKKEIVNESRQVLQEPDFAQSLLRDPNTPIIRKSRGTSTQGTSTHASSTQLAMVDDQRSKAGSIHSKVSSYHGSLHRSRDGRYTPCSYRGMEERLPHGSMSRLTDHSRHSSSHRLNEQSRHSSIRDLSNNPMTHITHGTSMNRVIEEDGTSA

SEQ ID NO: 62 - DNA, 2001 bp, Artificial Sequence
  cDNA
atggctatgacttggattgtcttctctctttggcccttgactgtgttcatggggcatataggtgggcacagtttgttttcttgtgaacctattaccttgaggatgtgccaagatttgccttataatactaccttcatgcctaatcttctgaatcattatgaccaacagacagcagctttggcaatggagccattccaccctatggtgaatctggattgttctcgggatttccggccttttctttgtgcactctacgctcctatttgtatggaatatggacgtgtcacacttccctgtcgtaggctgtgtcagcgggcttacagtgagtgttcgaagctcatggagatgtttggtgttccttggcctgaagatatggaatgcagtaggttcccagattgtgatgagccatatcctcgacttgtggatctgaatttagctggagaaccaactgaaggagccccagtggcagtgcagagagactatggtttttggtgtccccgagagttaaaaattgatcctgatctgggttattcttttctgcatgtgcgtgattgttcacctccttgtccaaatatgtacttcagaagagaagaactgtcatttgctcgctatttcataggattgatttcaatcatttgcctctcggccacattgtttacttttttaacttttttgattgatgtcacaagattccgttatcctgaaaggcctattatattttatgcagtctgctacatgatggtatccttaattttcttcattggatttttgcttgaagatcgagtagcctgcaatgcatccatccctgcacaatataaggcttccacagtgacacaaggatctcataataaagcctgtaccatgctttttatgatactctatttttttactatggctggcagtgtatggtgggtaattcttaccatcacatggtttttagcagctgtgccaaagtggggtagtgaagctattgagaagaaagcattgctgtttcacgccagtgcatggggcatccccggaactctaaccatcatccttttagcgatgaataaaattgaaggtgacaatattagtggcgtgtgttttgttggcctctacgatgttgatgcattgagatattttgttcttgctcccctctgcctgtatgtggtagttggggtttctctcctcttagctggcattatatccctaaacagagttcgaattgagattccattagaaaaggagaaccaagataaattagtgaagtttatgatccggatcggtgttttcagcattctttatctcgtaccactcttggttgtaattggatgctacttttatgagcaagcttaccggggcatctgggaaacaacgtggatacaagaacgctgcagagaatatcacattccatgtccatatcaggttactcaaatgagtcgtccagacttgattctctttctgatgaaatacctgatggctctcatagttggcattccctctgtattttgggttggaagcaaaaagacatgctttgaatgggccagtttttttcatggtcgtaggaaaaaagagatagtgaatgagagccgacaggtactccaggaacctgattttgctcagtctctcctgagggatccaaatactcctatcataagaaagtcaaggggaacttccactcaaggaacatccacccatgcttcttcaactcagctggctatggtggatgatcaaagaagcaaagcaggaagcatccacagcaaagtgagcagctaccacggcagcctccacagatcacgtgatggcaggtacacgccctgcagttacagaggaatggaggagagactacctcatggcagcatgtcacgactaacagatcactccaggcatagtagttctcatcggctcaatgaacagtcacgacatagcagcatcagagatctcagtaataatcccatgactcatatcacacatggcaccagcatgaatcgggttattgaagaagatggaaccagtgcttaa

SEQ ID NO: 63 - protein, 537 aa, Homo sapiens
MAWRGAGPSVPGAPGGVGLSLGLLLQLLLLLGPARGFGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEVPLPHKTPIQPGEECHSVGTNSDQYIWVKRSLNCVLKCGYDAGLYSRSAKEFTDIWMAVWASLCFISTAFTVLTFLIDSSRFSYPERPIIFLSMCYNIYSIAYIVRLTVGRERISCDFEEAAEPVLIQEGLKNTGCAIIFLLMYFFGMASSIWWVILTLTWFLAAGLKWGHEAIEMHSSYFHIAAWAIPAVKTIVILIMRLVDADELTGLCYVGNQNLDALTGFVVAPLFTYLVIGTLFIAAGLVALFKIRSNLQKDGTKTDKLERLMVKIGVFSVLYTVPATCVIACYFYEISNWALFRYSADDSNMAVEMLKIFMSLLVGITSGMWIWSAKTLHTWQKCSNRLVNSGKVKREKRGNGWVKPGKGSETVV

SEQ ID NO: 64 - DNA, 1614 bp, Artificial Sequence
  cDNA
atggcctggcggggcgcagggccgagcgtcccgggggcgcccgggggcgtcggtctcagtctggggttgctcctgcagttgctgctgctcctggggccggcgcggggcttcggggacgaggaagagcggcgctgcgaccccatccgcatctccatgtgccagaacctcggctacaacgtgaccaagatgcccaacctggttgggcacgagctgcagacggacgccgagctgcagctgacaactttcacaccgctcatccagtacggctgctccagccagctgcagttcttcctttgttctgtttatgtgccaatgtgcacagagaagatcaacatccccattggcccatgcggcggcatgtgtctttcagtcaagagacgctgtgaacccgtcctgaaggaatttggatttgcctggccagagagtctgaactgcagcaaattcccaccacagaacgaccacaaccacatgtgcatggaagggccaggtgatgaagaggtgcccttacctcacaaaacccccatccagcctggggaagagtgtcactctgtgggaaccaattctgatcagtacatctgggtgaaaaggagcctgaactgtgtgctcaagtgtggctatgatgctggcttatacagccgctcagccaaggagttcactgatatctggatggctgtgtgggccagcctgtgtttcatctccactgccttcacagtactgaccttcctgatcgattcttctaggttttcctaccctgagcgccccatcatatttctcagtatgtgctataatatttatagcattgcttatattgtcaggctgactgtaggccgggaaaggatatcctgtgattttgaagaggcagcagaacctgttctcatccaagaaggacttaagaacacaggatgtgcaataattttcttgctgatgtacttttttggaatggccagctccatttggtgggttattctgacactcacttggtttttggcagcaggactcaaatggggtcatgaagccattgaaatgcacagctcttatttccacattgcagcctgggccatccccgcagtgaaaaccattgtcatcttgattatgagactggtggatgcagatgaactgactggcttgtgctatgttggaaaccaaaatctcgatgccctcaccgggttcgtggtggctcccctctttacttatttggtcattggaactttgttcattgctgcaggtttggtggccttgttcaaaattcggtcaaatcttcaaaaggatgggacaaagacagacaagttagaaagactgatggtcaagattggggtgttctcagtactgtacacagttcctgcaacgtgtgtgattgcctgttatttttatgaaatctccaactgggcactttttcggtattctgcagatgattccaacatggctgttgaaatgttgaaaatttttatgtctttgttggtgggcatcacttcaggcatgtggatttggtctgccaaaactcttcacacgtggcagaagtgttccaacagattggtgaattctggaaaggtaaagagagagaagagaggaaatggttgggtgaagcctggaaaaggcagtgagactgtggtataa

SEQ ID NO: 65 - protein, 585 aa, Homo sapiens
MARPDPSAPPSLLLLLLAQLVGRAAAASKAPVCQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLCMDYNRSEATTAPPRPFPAKPTLPGPPGAPASGGECPAGGPFVCKCREPFVPILKESHPLYNKVRTGQVPNCAVPCYQPSFSADERTFATFWIGLWSVLCFISTSTTVATFLIDMERFRYPERPIIFLSACYLCVSLGFLVRLVVGHASVACSREHNHIHYETTGPALCTIVFLLVYFFGMASSIWWVILSLTWFLAAGMKWGNEAIAGYAQYFHLAAWLIPSVKSITALALSSVDGDPVAGICYVGNQNLNSLRGFVLGPLVLYLLVGTLFLLAGFVSLFRIRSVIKQGGTKTDKLEKLMIRIGIFTLLYTVPASIVVACYLYEQHYRESWEAALTCACPGHDTGQPRAKPEYWVLMLKYFMCLVVGITSGVWIWSGKTVESWRRFTSRCCCRPRRGHKSGGAMAAGDYPEASAALTGRTGPPGPAATYHKQVSLSHV

SEQ ID NO: 66 - DNA, 1758 bp, Artificial Sequence
  cNDA
atggctcggcctgacccatccgcgccgccctcgctgttgctgctgctcctagcgcagctggtgggccgggcggccgccgcgtccaaggccccggtgtgccaggaaatcacggtgcccatgtgccgcggcatcggctacaacctgacgcacatgcccaaccagttcaaccacgacacgcaggacgaggcgggcctggaggtgcaccagttctggccgctggtggagatccaatgctcgccggacctgcgcttcttcctatgctctatgtacacgcccatctgtctgcccgactaccacaagccgctgccgccctgccgctcggtgtgcgagcgcgccaaggccggctgctcgccgctgatgcgccagtacggcttcgcctggcccgagcgcatgagctgcgaccgcctcccggtgctgggccgcgacgccgaggtcctctgcatggattacaaccgcagcgaggccaccacggcgccccccaggcctttcccagccaagcccacccttccaggcccgccaggggcgccggcctcggggggcgaatgccccgctgggggcccgttcgtgtgcaagtgtcgcgagcccttcgtgcccattctgaaggagtcacacccgctctacaacaaggtgcggacgggccaggtgcccaactgcgcggtaccctgctaccagccgtccttcagtgccgacgagcgcacgttcgccaccttctggataggcctgtggtcggtgctgtgcttcatctccacgtccaccacagtggccaccttcctcatcgacatggaacgcttccgctatcctgagcgccccatcatcttcctgtcagcctgctacctgtgcgtgtcgctgggcttcctggtgcgtctggtcgtgggccatgccagcgtggcctgcagccgcgagcacaaccacatccactacgagaccacgggccctgcactgtgcaccatcgtcttcctcctggtctacttcttcggcatggccagctccatctggtgggtcatcctgtcgctcacctggttcctggccgccggcatgaagtggggcaacgaggccatcgcgggctacgcgcagtacttccacctggctgcgtggctcatccccagcgtcaagtccatcacggcactggcgctgagctccgtggacggggacccagtggccggcatctgctacgtgggcaaccagaacctgaactcgctgcgcggcttcgtgctgggcccgctggtgctctacctgctggtgggcacgctcttcctgctggcgggcttcgtgtcgctcttccgcatccgcagcgtcatcaagcagggcggcaccaagacggacaagctggagaagctcatgatccgcatcggcatcttcacgctgctctacacggtccccgccagcattgtggtggcctgctacctgtacgagcagcactaccgcgagagctgggaggcggcgctcacctgcgcctgcccgggccacgacaccggccagccgcgcgccaagcccgagtactgggtgctcatgctcaagtacttcatgtgcctggtggtgggcatcacgtcgggcgtctggatctggtcgggcaagacggtggagtcgtggcggcgtttcaccagccgctgctgctgccgcccgcggcgcggccacaagagcgggggcgccatggccgcaggggactaccccgaggcgagcgccgcgctcacaggcaggaccgggccgccgggccccgccgccacctaccacaagcaggtgtccctgtcgcacgtgtag

SEQ ID NO: 67 - protein, 706 aa, Homo sapiens
MEMFTFLLTCIFLPLLRGHSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLGPQKKTEQVQRDIGFWCPRHLKTSGGQGYKFLGIDQCAPPCPNMYFKSDELEFAKSFIGTVSIFCLCATLFTFLTFLIDVRRFRYPERPIIYYSVCYSIVSLMYFIGFLLGDSTACNKADEKLELGDTVVLGSQNKACTVLFMLLYFFTMAGTVWWVILTITWFLAAGRKWSCEAIEQKAVWFHAVAWGTPGFLTVMLLAMNKVEGDNISGVCFVGLYDLDASRYFVLLPLCLCVFVGLSLLLAGIISLNHVRQVIQHDGRNQEKLKKFMIRIGVFSGLYLVPLVTLLGCYVYEQVNRITWEITWVSDHCRQYHIPCPYQAKAKARPELALFMIKYLMTLIVGISAVFWVGSKKTCTEWAGFFKRNRKRDPISESRRVLQESCEFFLKHNSKVKHKKKHYKPSSHKLKVISKSMGTSTGATANHGTSAVAITSHDYLGQETLTEIQTSPETSMREVKADGASTPRLREQDCGEPASPAASISRLSGEQVDGKGQAGSVSESARSEGRISPKSDITDTGLAQSNNLQVPSSSEPSSLKGSTSLLVHPVSGVRKEQGGGCHSDT

SEQ ID NO: 68 - DNA, 2121 bp, Artificial Sequence
  cDNA
atggaaatgtttacatttttgttgacgtgtatttttctacccctcctaagagggcacagtctcttcacctgtgaaccaattactgttcccagatgtatgaaaatggcctacaacatgacgtttttccctaatctgatgggtcattatgaccagagtattgccgcggtggaaatggagcattttcttcctctcgcaaatctggaatgttcaccaaacattgaaactttcctctgcaaagcatttgtaccaacctgcatagaacaaattcatgtggttccaccttgtcgtaaactttgtgagaaagtatattctgattgcaaaaaattaattgacacttttgggatccgatggcctgaggagcttgaatgtgacagattacaatactgtgatgagactgttcctgtaacttttgatccacacacagaatttcttggtcctcagaagaaaacagaacaagtccaaagagacattggattttggtgtccaaggcatcttaagacttctgggggacaaggatataagtttctgggaattgaccagtgtgcgcctccatgccccaacatgtattttaaaagtgatgagctagagtttgcaaaaagttttattggaacagtttcaatattttgtctttgtgcaactctgttcacattccttacttttttaattgatgttagaagattcagatacccagagagaccaattatatattactctgtctgttacagcattgtatctcttatgtacttcattggatttttgctaggcgatagcacagcctgcaataaggcagatgagaagctagaacttggtgacactgttgtcctaggctctcaaaataaggcttgcaccgttttgttcatgcttttgtattttttcacaatggctggcactgtgtggtgggtgattcttaccattacttggttcttagctgcaggaagaaaatggagttgtgaagccatcgagcaaaaagcagtgtggtttcatgctgttgcatggggaacaccaggtttcctgactgttatgcttcttgctatgaacaaagttgaaggagacaacattagtggagtttgctttgttggcctttatgacctggatgcttctcgctactttgtactcttgccactgtgcctttgtgtgtttgttgggctctctcttcttttagctggcattatttccttaaatcatgttcgacaagtcatacaacatgatggccggaaccaagaaaaactaaagaaatttatgattcgaattggagtcttcagcggcttgtatcttgtgccattagtgacacttctcggatgttacgtctatgagcaagtgaacaggattacctgggagataacttgggtctctgatcattgtcgtcagtaccatatcccatgtccttatcaggcaaaagcaaaagctcgaccagaattggctttatttatgataaaatacctgatgacattaattgttggcatctctgctgtcttctgggttggaagcaaaaagacatgcacagaatgggctgggttttttaaacgaaatcgcaagagagatccaatcagtgaaagtcgaagagtactacaggaatcatgtgagtttttcttaaagcacaattctaaagttaaacacaaaaagaagcactataaaccaagttcacacaagctgaaggtcatttccaaatccatgggaaccagcacaggagctacagcaaatcatggcacttctgcagtagcaattactagccatgattacctaggacaagaaactttgacagaaatccaaacctcaccagaaacatcaatgagagaggtgaaagcggacggagctagcacccccaggttaagagaacaggactgtggtgaacctgcctcgccagcagcatccatctccagactctctggggaacaggtcgacgggaagggccaggcaggcagtgtatctgaaagtgcgcggagtgaaggaaggattagtccaaagagtgatattactgacactggcctggcacagagcaacaatttgcaggtccccagttcttcagaaccaagcagcctcaaaggttccacatctctgcttgttcacccggtttcaggagtgagaaaagagcagggaggtggttgtcattcagatacttga

SEQ ID NO: 69 - protein, 574 aa, Homo sapiens
MRDPGAAAPLSSLGLCALVLALLGALSAGAGAQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGPGGGPTAYPTAPYLPDLPFTALPPGASDGRGRPAFPFSCPRQLKVPPYLGYRFLGERDCGAPCEPGRANGLMYFKEEERRFARLWVGVWSVLCCASTLFTVLTYLVDMRRFSYPERPIIFLSGCYFMVAVAHVAGFLLEDRAVCVERFSDDGYRTVAQGTKKEGCTILFMVLYFFGMASSIWWVILSLTWFLAAGMKWGHEAIEANSQYFHLAAWAVPAVKTITILAMGQVDGDLLSGVCYVGLSSVDALRGFVLAPLFVYLFIGTSFLLAGFVSLFRIRTIMKHDGTKTEKLEKLMVRIGVFSVLYTVPATIVLACYFYEQAFREHWERTWLLQTCKSYAVPCPPGHFPPMSPDFTVFMIKYLMTMIVGITTGFWIWSGKTLQSWRRFYHRLSHSSKGETAV

SEQ ID NO: 70 - DNA, 1725 bp, Artificial Sequence
  cDNA
atgcgggaccccggcgcggccgctccgctttcgtccctgggcctctgtgccctggtgctggcgctgctgggcgcactgtccgcgggcgccggggcgcagccgtaccacggagagaagggcatctccgtgccggaccacggcttctgccagcccatctccatcccgctgtgcacggacatcgcctacaaccagaccatcctgcccaacctgctgggccacacgaaccaagaggacgcgggcctcgaggtgcaccagttctacccgctggtgaaggtgcagtgttctcccgaactccgctttttcttatgctccatgtatgcgcccgtgtgcaccgtgctcgatcaggccatcccgccgtgtcgttctctgtgcgagcgcgcccgccagggctgcgaggcgctcatgaacaagttcggcttccagtggcccgagcggctgcgctgcgagaacttcccggtgcacggtgcgggcgagatctgcgtgggccagaacacgtcggacggctccgggggcccaggcggcggccccactgcctaccctaccgcgccctacctgccggacctgcccttcaccgcgctgcccccgggggcctcagatggcagggggcgtcccgccttccccttctcatgcccccgtcagctcaaggtgcccccgtacctgggctaccgcttcctgggtgagcgcgattgtggcgccccgtgcgaaccgggccgtgccaacggcctgatgtactttaaggaggaggagaggcgcttcgcccgcctctgggtgggcgtgtggtccgtgctgtgctgcgcctcgacgctctttaccgttctcacctacctggtggacatgcggcgcttcagctacccagagcggcccatcatcttcctgtcgggctgctacttcatggtggccgtggcgcacgtggccggcttccttctagaggaccgcgccgtgtgcgtggagcgcttctcggacgatggctaccgcacggtggcgcagggcaccaagaaggagggctgcaccatcctcttcatggtgctctacttcttcggcatggccagctccatctggtgggtcattctgtctctcacttggttcctggcggccggcatgaagtggggccacgaggccatcgaggccaactcgcagtacttccacctggccgcgtgggccgtgcccgccgtcaagaccatcactatcctggccatgggccaggtagacggggacctgctgagcggggtgtgctacgttggcctctccagtgtggacgcgctgcggggcttcgtgctggcgcctctgttcgtctacctcttcataggcacgtccttcttgctggccggcttcgtgtccctcttccgtatccgcaccatcatgaaacacgacggcaccaagaccgagaagctggagaagctcatggtgcgcatcggcgtcttcagcgtgctctacacagtgcccgccaccatcgtcctggcctgctacttctacgagcaggccttccgcgagcactgggagcgcacctggctcctgcagacgtgcaagagctatgccgtgccctgcccgcccggccacttcccgcccatgagccccgacttcaccgtcttcatgatcaagtacctgatgaccatgatcgtcggcatcaccactggcttctggatctggtcgggcaagaccctgcagtcgtggcgccgcttctaccacagacttagccacagcagcaagggggagactgcggtatga

SEQ ID NO: 71 - protein, 694 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGAGGAGGAAAGAGAAGAGAGGPGGRGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQDGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRSLCTRCCWASKGAAVGGGAGATAAGGGGGPGGGGGGGPGGGGGPGGGGGSLYSDVSTGLTWRSGTASSVSYPKQMPLSQV

SEQ ID NO: 72 - DNA, 2085 bp, Artificial Sequence
  cDNA
atggagtggggttacctgttggaagtgacctcgctgctggccgccttggcgctgctgcagcgctctagcggcgctgcggccgcctcggccaaggagctggcatgccaagagatcaccgtgccgctgtgtaagggcatcggctacaactacacctacatgcccaatcagttcaaccacgacacgcaagacgaggcgggcctggaggtgcaccagttctggccgctggtggagatccagtgctcgcccgatctcaagttcttcctgtgcagcatgtacacgcccatctgcctagaggactacaagaagccgctgccgccctgccgctcggtgtgcgagcgcgccaaggccggctgcgcgccgctcatgcgccagtacggcttcgcctggcccgaccgcatgcgctgcgaccggctgcccgagcaaggcaaccctgacacgctgtgcatggactacaaccgcaccgacctaaccaccgccgcgcccagcccgccgcgccgcctgccgccgccgccgcccggcgagcagccgccttcgggcagcggccacggccgcccgccgggggccaggcccccgcaccgcggcggcggcaggggcggtggcggcggggacgcggcggcgcccccagctcgcggcggcggcggtggcgggaaggcgcggccccctggcggcggcgcggctccctgcgagcccgggtgccagtgccgcgcgcctatggtgagcgtgtccagcgagcgccacccgctctacaaccgcgtcaagacaggccagatcgctaactgcgcgctgccctgccacaacccctttttcagccaggacgagcgcgccttcaccgtcttctggatcggcctgtggtcggtgctctgcttcgtgtccaccttcgccaccgtctccaccttccttatcgacatggagcgcttcaagtacccggagcggcccattatcttcctctcggcctgctacctcttcgtgtcggtgggctacctagtgcgcctggtggcgggccacgagaaggtggcgtgcagcggtggcgcgccgggcgcggggggcgctgggggcgcgggcggcgcggcggcgggcgcgggcgcggcgggcgcgggcgcgggcggcccgggcgggcgcggcgagtacgaggagctgggcgcggtggagcagcacgtgcgctacgagaccaccggccccgcgctgtgcaccgtggtcttcttgctggtctacttcttcggcatggccagctccatctggtgggtgatcttgtcgctcacatggttcctggcggccggtatgaagtggggcaacgaagccatcgccggctactcgcagtacttccacctggccgcgtggcttgtgcccagcgtcaagtccatcgcggtgctggcgctcagctcggtggacggcgacccggtggcgggcatctgctacgtgggcaaccagagcctggacaacctgcgcggcttcgtgctggcgccgctggtcatctacctcttcatcggcaccatgttcctgctggccggcttcgtgtccctcttccgcatccgctcggtcatcaagcaacaggacggccccaccaagacgcacaagctggagaagctgatgatccgcctgggcctgttcaccgtgctctacaccgtgcccgccgcggtggtggtcgcctgcctcttctacgagcagcacaaccgcccgcgctgggaggccacgcacaactgcccgtgcctgcgggacctgcagcccgaccaggcacgcaggcccgactacgccgtcttcatgctcaagtacttcatgtgcctagtggtgggcatcacctcgggcgtgtgggtctggtccggcaagacgctggagtcctggcgctccctgtgcacccgctgctgctgggccagcaagggcgccgcggtgggcgggggcgcgggcgccacggccgcggggggtggcggcgggccggggggcggcggcggcgggggacccggcggcggcggggggccgggcggcggcgggggctccctctacagcgacgtcagcactggcctgacgtggcggtcgggcacggcgagctccgtgtcttatccaaagcagatgccattgtcccaggtctga

SEQ ID NO: 73 - protein, 591 aa, Homo sapiens
MAVAPLRGALLLWQLLAAGGAALEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENATAGPAEPHKGLGMLPVAPRPARPPGDLGPGAGGSGTCENPEKFQYVEKSRSCAPRCGPGVEVFWSRRDKDFALVWMAVWSALCFFSTAFTVLTFLLEPHRFQYPERPIIFLSMCYNVYSLAFLIRAVAGAQSVACDQEAGALYVIQEGLENTGCTLVFLLLYYFGMASSLWWVVLTLTWFLAAGKKWGHEAIEAHGSYFHMAAWGLPALKTIVILTLRKVAGDELTGLCYVASTDAAALTGFVLVPLSGYLVLGSSFLLTGFVALFHIRKIMKTGGTNTEKLEKLMVKIGVFSILYTVPATCVIVCYVYERLNMDFWRLRATEQPCAAAAGPGGRRDCSLPGGSVPTVAVFMLKIFMSLVVGITSGVWVWSSKTFQTWQSLCYRKIAAGRARAKACRAPGSYGRGTHCHYKAPTVVLHMTKTDPSLENPTHL

SEQ ID NO: 74 - DNA, 1776 bp, Artificial Sequence
  cDNA
atggccgtggcgcctctgcggggggcgctgctgctgtggcagctgctggcggcgggcggcgcggcactggagatcggccgcttcgacccggagcgcgggcgcggggctgcgccgtgccaggcggtggagatccccatgtgccgcggcatcggctacaacctgacccgcatgcccaacctgctgggccacacgtcgcagggcgaggcggctgccgagctagcggagttcgcgccgctggtgcagtacggctgccacagccacctgcgcttcttcctgtgctcgctctacgcgcccatgtgcaccgaccaggtctcgacgcccattcccgcctgccggcccatgtgcgagcaggcgcgcctgcgctgcgcgcccatcatggagcagttcaacttcggctggccggactcgctcgactgcgcccggctgcccacgcgcaacgacccgcacgcgctgtgcatggaggcgcccgagaacgccacggccggccccgcggagccccacaagggcctgggcatgctgcccgtggcgccgcggcccgcgcgccctcccggagacctgggcccgggcgcgggcggcagtggcacctgcgagaaccccgagaagttccagtacgtggagaagagccgctcgtgcgcaccgcgctgcgggcccggcgtcgaggtgttctggtcccggcgcgacaaggacttcgcgctggtctggatggccgtgtggtcggcgctgtgcttcttctccaccgccttcactgtgctcaccttcttgctggagccccaccgcttccagtaccccgagcgccccatcatcttcctctccatgtgctacaacgtctactcgctggccttcctgatccgtgcggtggccggagcgcagagcgtggcctgtgaccaggaggcgggcgcgctctacgtgatccaggagggcctggagaacacgggctgcacgctggtcttcctactgctctactacttcggcatggccagctcgctctggtgggtggtcctgacgctcacctggttcctggctgccgggaagaaatggggccacgaggccatcgaggcccacggcagctatttccacatggctgcctggggcctgcccgcgctcaagaccatcgtcatcctgaccctgcgcaaggtggcgggtgatgagctgactgggctttgctacgtggccagcacggatgcagcagcgctcacgggcttcgtgctggtgcccctctctggctacctggtgctgggcagtagtttcctcctgaccggcttcgtggccctcttccacatccgcaagatcatgaagacgggcggcaccaacacagagaagctggagaagctcatggtcaagatcggggtcttctccatcctctacacggtgcccgccacctgcgtcatcgtttgctatgtctacgaacgcctcaacatggacttctggcgccttcgggccacagagcagccatgcgcagcggccgcggggcccggaggccggagggactgctcgctgccagggggctcggtgcccaccgtggcggtcttcatgctcaaaattttcatgtcactggtggtggggatcaccagcggcgtctgggtgtggagctccaagactttccagacctggcagagcctgtgctaccgcaagatagcagctggccgggcccgggccaaggcctgccgcgcccccgggagctacggacgtggcacgcactgccactataaggctcccaccgtggtcttgcacatgactaagacggacccctctttggagaaccccacacacctctag

SEQ ID NO: 75 - protein, 581 aa, Homo sapiens
MQRPGPRLWLVLQVMGSCAAISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNGSDEPTRGSGLFPPLFRPQRPHSAQEHPLKDGGPGRGGCDNPGKFHHVEKSASCAPLCTPGVDVYWSREDKRFAVVWLAIWAVLCFFSSAFTVLTFLIDPARFRYPERPIIFLSMCYCVYSVGYLIRLFAGAESIACDRDSGQLYVIQEGLESTGCTLVFLVLYYFGMASSLWWVVLTLTWFLAAGKKWGHEAIEANSSYFHLAAWAIPAVKTILILVMRRVAGDELTGVCYVGSMDVNALTGFVLIPLACYLVIGTSFILSGFVALFHIRRVMKTGGENTDKLEKLMVRIGLFSVLYTVPATCVIACYFYERLNMDYWKILAAQHKCKMNNQTKTLDCLMAASIPAVEIFMVKIFMLLVVGITSGMWIWTSKTLQSWQQVCSRRLKKKSRRKPASVITSGGIYKKAQHPQKTHHGKYEIPAQSPTCV

SEQ ID NO: 76 - DNA, 1746 bp, Artificial Sequence
  cDNA
atgcagcgcccgggcccccgcctgtggctggtcctgcaggtgatgggctcgtgcgccgccatcagctccatggacatggagcgcccgggcgacggcaaatgccagcccatcgagatcccgatgtgcaaggacatcggctacaacatgactcgtatgcccaacctgatgggccacgagaaccagcgcgaggcagccatccagttgcacgagttcgcgccgctggtggagtacggctgccacggccacctccgcttcttcctgtgctcgctgtacgcgccgatgtgcaccgagcaggtctctacccccatccccgcctgccgggtcatgtgcgagcaggcccggctcaagtgctccccgattatggagcagttcaacttcaagtggcccgactccctggactgccggaaactccccaacaagaacgaccccaactacctgtgcatggaggcgcccaacaacggctcggacgagcccacccggggctcgggcctgttcccgccgctgttccggccgcagcggccccacagcgcgcaggagcacccgctgaaggacgggggccccgggcgcggcggctgcgacaacccgggcaagttccaccacgtggagaagagcgcgtcgtgcgcgccgctctgcacgcccggcgtggacgtgtactggagccgcgaggacaagcgcttcgcagtggtctggctggccatctgggcggtgctgtgcttcttctccagcgccttcaccgtgctcaccttcctcatcgacccggcccgcttccgctaccccgagcgccccatcatcttcctctccatgtgctactgcgtctactccgtgggctacctcatccgcctcttcgccggcgccgagagcatcgcctgcgaccgggacagcggccagctctatgtcatccaggagggactggagagcaccggctgcacgctggtcttcctggtcctctactacttcggcatggccagctcgctgtggtgggtggtcctcacgctcacctggttcctggccgccggcaagaagtggggccacgaggccatcgaagccaacagcagctacttccacctggcagcctgggccatcccggcggtgaagaccatcctgatcctggtcatgcgcagggtggcgggggacgagctcaccggggtctgctacgtgggcagcatggacgtcaacgcgctcaccggcttcgtgctcattcccctggcctgctacctggtcatcggcacgtccttcatcctctcgggcttcgtggccctgttccacatccggagggtgatgaagacgggcggcgagaacacggacaagctggagaagctcatggtgcgtatcgggctcttctctgtgctgtacaccgtgccggccacctgtgtgatcgcctgctacttttacgaacgcctcaacatggattactggaagatcctggcggcgcagcacaagtgcaaaatgaacaaccagactaaaacgctggactgcctgatggccgcctccatccccgccgtggagatcttcatggtgaagatctttatgctgctggtggtggggatcaccagcgggatgtggatttggacctccaagactctgcagtcctggcagcaggtgtgcagccgtaggttaaagaagaagagccggagaaaaccggccagcgtgatcaccagcggtgggatttacaaaaaagcccagcatccccagaaaactcaccacgggaaatatgagatccctgcccagtcgcccacctgcgtgtga

SEQ ID NO: 77 - protein, 1615 aa, Homo sapiens [sequence omitted]

SEQ ID NO: 78 - DNA, 4848 bp, Artificial Sequence
  cDNA
atggaggcagcgccgcccgggccgccgtggccgctgctgctgctgctgctgctgctgctggcgctgtgcggctgcccggcccccgccgcggcctcgccgctcctgctatttgccaaccgccgggacgtacggctggtggacgccggcggagtcaagctggagtccaccatcgtggtcagcggcctggaggatgcggccgcagtggacttccagttttccaagggagccgtgtactggacagacgtgagcgaggaggccatcaagcagacctacctgaaccagacgggggccgccgtgcagaacgtggtcatctccggcctggtctctcccgacggcctcgcctgcgactgggtgggcaagaagctgtactggacggactcagagaccaaccgcatcgaggtggccaacctcaatggcacatcccggaaggtgctcttctggcaggaccttgaccagccgagggccatcgccttggaccccgctcacgggtacatgtactggacagactggggtgagacgccccggattgagcgggcagggatggatggcagcacccggaagatcattgtggactcggacatttactggcccaatggactgaccatcgacctggaggagcagaagctctactgggctgacgccaagctcagcttcatccaccgtgccaacctggacggctcgttccggcagaaggtggtggagggcagcctgacgcaccccttcgccctgacgctctccggggacactctgtactggacagactggcagacccgctccatccatgcctgcaacaagcgcactggggggaagaggaaggagatcctgagtgccctctactcacccatggacatccaggtgctgagccaggagcggcagcctttcttccacactcgctgtgaggaggacaatggcggctgctcccacctgtgcctgctgtccccaagcgagcctttctacacatgcgcctgccccacgggtgtgcagctgcaggacaacggcaggacgtgtaaggcaggagccgaggaggtgctgctgctggcccggcggacggacctacggaggatctcgctggacacgccggacttcaccgacatcgtgctgcaggtggacgacatccggcacgccattgccatcgactacgacccgctagagggctatgtctactggacagatgacgaggtgcgggccatccgcagggcgtacctggacgggtctggggcgcagacgctggtcaacaccgagatcaacgaccccgatggcatcgcggtcgactgggtggcccgaaacctctactggaccgacacgggcacggaccgcatcgaggtgacgcgcctcaacggcacctcccgcaagatcctggtgtcggaggacctggacgagccccgagccatcgcactgcaccccgtgatgggcctcatgtactggacagactggggagagaaccctaaaatcgagtgtgccaacttggatgggcaggagcggcgtgtgctggtcaatgcctccctcgggtggcccaacggcctggccctggacctgcaggaggggaagctctactggggagacgccaagacagacaagatcgaggtgatcaatgttgatgggacgaagaggcggaccctcctggaggacaagctcccgcacattttcgggttcacgctgctgggggacttcatctactggactgactggcagcgccgcagcatcgagcgggtgcacaaggtcaaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacgtggccatcccgctcacgggcgtcaaggaggcctcagccctggactttgatgtgtccaacaaccacatttactggacagacgtcagcctgaagaccatcagccgcgccttcatgaacgggagctcggtggagcacgtggtggagtttggccttgactaccccgagggcatggccgttgactggatgggcaagaacctctactgggccgacactgggaccaatagaatcgaagtggcgcggctggacgggcagttccggcaagtcctcgtgtggagggacttggacaacccgaggtcgctggccctggatcccaccaagggctacatctactggaccgagtggggcggcaagccgaggatcgtgcgggccttcatggacgggaccaactgcatgacgctggtggacaaggtgggccgggccaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccggacgaccagcacagcccggatctcatcctgcccctgcatggactgaggaacgtcaaagccatcgactatgacccactggacaagttcatctactgggtggatgggcgccagaacatcaagcgagccaaggacgacgggacccagccctttgttttgacctctctgagccaaggccaaaacccagacaggcagccccacgacctcagcatcgacatctacagccggacactgttctggacgtgcgaggccaccaataccatcaacgtccacaggctgagcggggaagccatgggggtggtgctgcgtggggaccgcgacaagcccagggccatcgtcgtcaacgcggagcgagggtacctgtacttcaccaacatgcaggaccgggcagccaagatcgaacgcgcagccctggacggcaccgagcgcgaggtcctcttcaccaccggcctcatccgccctgtggccctggtggtggacaacacactgggcaagctgttctgggtggacgcggacctgaagcgcattgagagctgtgacctgtcaggggccaaccgcctgaccctggaggacgccaacatcgtgcagcctctgggcctgaccatccttggcaagcatctctactggatcgaccgccagcagcagatgatcgagcgtgtggagaagaccaccggggacaagcggactcgcatccagggccgtgtcgcccacctcactggcatccatgcagtggaggaagtcagcctggaggagttctcagcccacccatgtgcccgtgacaatggtggctgctcccacatctgtattgccaagggtgatgggacaccacggtgctcatgcccagtccacctcgtgctcctgcagaacctgctgacctgtggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggtggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgcgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctga

SEQ ID NO: 79 - protein, 1613 aa, Homo sapiens
MGAVLRSLLACSFCVLLRAAPLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSVGVVLKGEQDRPRAVVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 80 - DNA, 4842 bp, Artificial Sequence
  cDNA
atgggggccgtcctgaggagcctcctggcctgcagcttctgtgtgctcctgagagcggcccctttgttgctttatgcaaacagacgggacttgcgattggttgatgctacaaatggcaaagagaatgctacgattgtagttggaggcttggaggatgcagctgcggtggactttgtgtttagtcatggcttgatatactggagtgatgtcagcgaagaagccattaaacgaacagaatttaacaaaactgagagtgtgcagaatgttgttgtttctggattattgtcccccgatgggctggcatgtgattggcttggagaaaaattgtactggacagattctgaaactaatcggattgaagtttctaatttagatggatctttacgaaaagttttattttggcaagagttggatcaacccagagctattgccttagatccttcaagtgggttcatgtactggacagactggggagaagtgccaaagatagaacgtgctggaatggatggttcaagtcgcttcattataataaacagtgaaatttactggccaaatggactgactttggattatgaagaacaaaagctttattgggcagatgcaaaacttaatttcatccacaaatcaaatctggatggaacaaatcggcaggcagtggttaaaggttcccttccacatccttttgccttgacgttatttgaggacatattgtactggactgactggagcacacactccattttggcttgcaacaagtatactggtgagggtctgcgtgaaatccattctgacatcttctctcccatggatatacatgccttcagccaacagaggcagccaaatgccacaaatccatgtggaattgacaatgggggttgttcccatttgtgtttgatgtctccagtcaagcctttttatcagtgtgcttgccccactggggtcaaactcctggagaatggaaaaacctgcaaagatggtgccacagaattattgcttttagctcgaaggacagacttgagacgcatttctttggatacaccagattttacagacattgttctgcagttagaagacatccgtcatgccattgccatagattacgatcctgtggaaggctacatctactggactgatgatgaagtgagggccatacgccgttcatttatagatggatctggcagtcagtttgtggtcactgctcaaattgcccatcctgatggtattgctgtggactgggttgcacgaaatctttattggacagacactggcactgatcgaatagaagtgacaaggctcaatgggaccatgaggaagatcttgatttcagaggacttagaggaaccccgggctattgtgttagatcccatggttgggtacatgtattggactgactggggagaaattccgaaaattgagcgagcagctctggatggttctgaccgtgtagtattggttaacacttctcttggttggccaaatggtttagccttggattatgatgaaggcaaaatatactggggagatgccaaaacagacaagattgaggttatgaatactgatggcactgggagacgagtactagtggaagacaaaattcctcacatatttggatttactttgttgggtgactatgtttactggactgactggcagaggcgtagcattgaaagagttcataaacgaagtgcagagagggaagtgatcatagatcagctgcctgacctcatgggcctaaaggctacaaatgttcatcgagtgattggttccaacccctgtgctgaggaaaacgggggatgtagccatctctgcctctatagacctcagggccttcgctgtgcttgccctattggctttgaactcatcagtgacatgaagacctgcattgtcccagaggctttccttttgttttcacggagagcagatatcagacgaatttctctggaaacaaacaataataatgtggctattccactcactggtgtcaaagaagcttctgctttggattttgatgtgacagacaaccgaatttattggactgatatatcactcaagaccatcagcagagcctttatgaatggcagtgcactggaacatgtggtagaattcggcttagattatccagaaggcatggcagtagactggcttgggaagaacttgtactgggcagacacaggaacgaatcgaattgaggtgtcaaagttggatgggcagcaccgacaagttttggtgtggaaagacctagatagtcccagagctctcgcgttggaccctgccgaaggatttatgtattggactgaatggggtggaaaacctaagatagacagagctgcaatggatggaagtgaacgtactaccttagttccaaatgtggggcgggcaaacggcctaactattgattatgctaaaaggaggctttattggacagacctggacaccaacttaatagaatcttcaaatatgcttgggctcaaccgtgaagttatagcagatgacttgcctcatccttttggcttaactcagtaccaagattatatctactggacggactggagccgacgcagcattgagcgtgccaacaaaaccagtggccaaaaccgcaccatcattcagggccatttggattatgtgatggacatcctcgtctttcactcatctcgacagtcagggtggaatgaatgtgcttccagcaatgggcactgctcccacctctgcttggctgtgccagttgggggttttgtttgtggatgccctgcccactactctcttaatgctgacaacaggacttgtagtgctcctacgactttcctgctcttcagtcaaaagagtgccatcaaccgcatggtgattgatgaacaacagagccccgacatcatccttcccatccacagccttcggaatgtccgggccattgactatgacccactggacaagcaactctattggattgactcacgacaaaacatgatccgaaaggcacaagaagatggcagccagggctttactgtggttgtgagctcagttccgagtcagaacctggaaatacaaccctatgacctcagcattgatatttacagccgctacatctactggacttgtgaggctaccaatgtcattaatgtgacaagattagatgggagatcagttggagtggtgctgaaaggcgagcaggacagacctcgagccgttgtggtaaacccagagaaagggtatatgtattttaccaatcttcaggaaaggtctcctaaaattgaacgggctgctttggatgggacagaacgggaggtcctctttttcagtggcttaagtaaaccaattgctttagcccttgatagcaggctgggcaagctcttttgggctgattcagatctccggcgaattgaaagcagtgatctctcaggtgctaaccggatagtattagaagactccaatatcttgcagcctgtgggacttactgtgtttgaaaactggctctattggattgataaacagcagcaaatgattgaaaaaattgacatgacaggtcgagagggtagaaccaaagtccaagctcgaattgcccagcttagtgacattcatgcagtaaaggagctgaaccttcaagaatacagacagcacccttgtgctcaggataatggtggctgttcacatatttgtcttgtaaagggggatggtactacaaggtgttcttgccccatgcacctggttctacttcaagatgagctatcatgtggagaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctga

SEQ ID NO: 81 - protein, 11 aa, artificial sequence
  linker
SPKKKRKVEAS

SEQ ID NO: 82 - protein, 16 aa, artificial sequence
  linker
SGSETPGTSESATPES

SEQ ID NO: 83 - protein, 17 aa, artificial sequence
  linker
KSGSETPGTSESATPES

SEQ ID NO: 84 - protein, 638 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 85 - DNA, 1917 bp, artificial sequence
  cDNA
atggatctctggcagctgctgttgaccttggcactggcaggatcaagtgatgctttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtgcgtgtgagatccaaacaacgaaactctggaaattatggcgagttcagtgaggtgctctatgtaacacttcctcagatgagccaatttacatgtgaagaagatttctactttccatggctcttaattattatctttggaatatttgggctaacagtgatgctatttgtattcttattttctaaacagcaaaggattaaaatgctgattctgcccccagttccagttccaaagattaaaggaatcgatccagatctcctcaaggaaggaaaattagaggaggtgaacacaatcttagccattcatgatagctataaacccgaattccacagtgatgactcttgggttgaatttattgagctagatattgatgagccagatgaaaagactgaggaatcagacacagacagacttctaagcagtgaccatgagaaatcacatagtaacctaggggtgaaggatggcgactctggacgtaccagctgttgtgaacctgacattctggagactgatttcaatgccaatgacatacatgagggtacctcagaggttgctcagccacagaggttaaaaggggaagcagatctcttatgccttgaccagaagaatcaaaataactcaccttatcatgatgcttgccctgctactcagcagcccagtgttatccaagcagagaaaaacaaaccacaaccacttcctactgaaggagctgagtcaactcaccaagctgcccatattcagctaagcaatccaagttcactgtcaaacatcgacttttatgcccaggtgagcgacattacaccagcaggtagtgtggtcctttccccgggccaaaagaataaggcagggatgtcccaatgtgacatgcacccggaaatggtctcactctgccaagaaaacttccttatggacaatgcctacttctgtgaggcagatgccaaaaagtgcatccctgtggctcctcacatcaaggttgaatcacacatacagccaagcttaaaccaagaggacatttacatcaccacagaaagccttaccactgctgctgggaggcctgggacaggagaacatgttccaggttctgagatgcctgtcccagactatacctccattcatatagtacagtccccacagggcctcatactcaatgcgactgccttgcccttgcctgacaaagagtttctctcatcatgtggctatgtgagcacagaccaactgaacaaaatcatgccttag

SEQ ID NO: 86 - protein, 288 aa, homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 87 - DNA, 867 bp, Artificial sequence
  cDNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 88 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 89 - DNA, 873 bp, Artificial sequence
  cDNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 90 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 91 - DNA, 672 bp, Artificial sequence
  cDNA
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 92 - protein, 183 aa, Homo sapiens
MENGAVYSPTTEEDPGPARGPRSGLAAYFFMGRLPLLRRVLKGLQLLLSLLAFICEEVVSQCTLCGGLYFFEFVSCSAFLLSLLILIVYCTPFYERVDTTKVKSSDFYITLGTGCVFLLASIIFVSTHDRTSAEIAAIVFGFIASFMFLLDFITMLYEKRQESQLRKPENTTRAEALTEPLNA

SEQ ID NO: 93 - DNA, 552 bp, Artificial sequence
  cDNA
atggagaacggagcggtgtacagccccactacggaggaggacccgggccccgccagaggcccccggagcggcctcgctgcctactttttcatgggccggctcccattgctccggcgcgttctcaagggcttgcagctgttgctgtctctgctggccttcatctgtgaagaagttgtatcacaatgtactttatgtggaggactttatttttttgagtttgtaagctgcagtgcctttcttctgagtctccttatactgattgtgtattgcactccattttatgagagagttgataccacaaaagtaaaatcatcggatttttatattactttgggaacaggatgtgtgtttttgttggcatccatcatttttgtttccacacatgacaggacttcagctgagattgctgcaattgtgtttggatttatagcaagttttatgttcctacttgactttatcactatgctgtatgaaaaacgacaggagtcccagctgagaaaacctgaaaataccactagggctgaagccctcactgagccacttaatgcctaa

SEQ ID NO: 94 - protein, 234 aa, Homo sapiens
MRSGEELDGFEGEASSTSMISGASSPYQPTTEPVSQRRGLAGLRCDPDYLRGALGRLKVAQVILALIAFICIETIMACSPCEGLYFFEFVSCSAFVVTGVLLIMFSLNLHMRIPQINWNLTDLVNTGLSAFLFFIASIVLAALNHRAGAEIAAVIFGFLATAAYAVNTFLAVQKWRVSVRQQSTNDYIRARTESRDVDSRPEIQRLDTFSYSTNVTVRKKSPTNLLSLNHWQLA

SEQ ID NO: 95 - DNA, 627 bp, Artificial sequence
  cDNA
atgcggagcggcgaggagctggacggcttcgagggcgaggcctcgagcacctccatgatctcgggcgccagcagcccgtaccagcccaccaccgagccggtgagccagcgccgcgggctggccggcctgcgctgcgaccccgactacctgcgcggcgcgctcggccgcctcaaggtcgcccaagtgatcttggccctgattgcattcatctgcatagagaccatcatggcatgctccccgtgtgaaggcctctacttttttgagtttgtgagctgcagtgcgtttgtggtgactggcgtcttgctgattatgttcagtctcaacctgcacatgaggatcccccagatcaactggaatctgacagatttggtcaacactggactcagcgctttccttttctttattgcttcaatcgtactggctgctttaaaccatagagccggagcagaaattgctgccgtgatatttggcttcttggcgactgcggcatatgcagtgaacacattcctggcagtgcagaaatggagagtcagcgtccgccagcagagcaccaatgactacatccgagcccgcacggagtccagggatgtggacagtcgccctgagatccagcgcctggacacgtga

SEQ ID NO: 96 - protein, 16 aa, Artificial sequence
  alpha tag
MPSRLEEELRRRLTEP

SEQ ID NO: 97 - protein, 14 aa, Artificial sequence
  E6 tag
QADQEAKELARQIS

SEQ ID NO: 98 - protein, 122 aa, Artificial sequence
  anti-alpha VHH
EVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 99 - protein, 18 aa, Artificial sequence
  CDR3 anti-E6 VHH
VYYCSKSGAYWGQGTQVT

SEQ ID NO: 100 - protein, 541 aa, Homo sapiens
MAGAIIENMSTKKLCIVGGILLVFQIIAFLVGGLIAPGPTTAVSYMSVKCVDARKNHHKTKWFVPWGPNHCDKIRDIEEAIPREIEANDIVFSVHIPLPHMEMSPWFQFMLFILQLDIAFKLNNQIRENAEVSMDVSLAYRDDAFAEWTEMAHERVPRKLKCTFTSPKTPEHEGRYYECDVLPFMEIGSVAHKFYLLNIRLPVNEKKKINVGIGEIKDIRLVGIHQNGGFTKVWFAMKTFLTPSIFIIMVWYWRRITMMSRPPVLLEKVIFALGISMTFINIPVEWFSIGFDWTWMLLFGDIRQGIFYAMLLSFWIIFCGEHMMDQHERNHIAGYWKQVGPIAVGSFCLFIFDMCERGVQLTNPFYSIWTTDIGTELAMAFIIVAGICLCLYFLFLCFMVFQVFRNISGKQSSLPAMSKVRRLHYEGLIFRFKFLMLITLACAAMTVIFFIVSQVTEGHWKWGGVTVQVNSAFFTGIYGMWNLYVFALMFLYAPSHKNYGEDQSNGDLGVHSGEELQLTTTITHVDGPTEIYKLTRKEAQE

SEQ ID NO: 101 - DNA, 1626 bp, Artificial sequence
  cDNA
atggctggggcaattatagaaaacatgagcaccaagaagctgtgcattgttggtgggattctgctcgtgttccaaatcatcgcctttctggtgggaggcttgattgctccagggcccacaacggcagtgtcctacatgtcggtgaaatgtgtggatgcccgtaagaaccatcacaagacaaaatggttcgtgccttggggacccaatcattgtgacaagatccgagacattgaagaggcaattccaagggaaattgaagccaatgacatcgtgttttctgttcacattcccctcccccacatggagatgagtccttggttccaattcatgctgtttatcctgcagctggacattgccttcaagctaaacaaccaaatcagagaaaatgcagaagtctccatggacgtttccctggcttaccgtgatgacgcgtttgctgagtggactgaaatggcccatgaaagagtaccacggaaactcaaatgcaccttcacatctcccaagactccagagcatgagggccgttactatgaatgtgatgtccttcctttcatggaaattgggtctgtggcccataagttttaccttttaaacatccggctgcctgtgaatgagaagaagaaaatcaatgtgggaattggggagataaaggatatccggttggtggggatccaccaaaatggaggcttcaccaaggtgtggtttgccatgaagaccttccttacgcccagcatcttcatcattatggtgtggtattggaggaggatcaccatgatgtcccgacccccagtgcttctggaaaaagtcatctttgcccttgggatttccatgacctttatcaatatcccagtggaatggttttccatcgggtttgactggacctggatgctgctgtttggtgacatccgacagggcatcttctatgcgatgcttctgtccttctggatcatcttctgtggcgagcacatgatggatcagcacgagcggaaccacatcgcagggtattggaagcaagtcggacccattgccgttggctccttctgcctcttcatatttgacatgtgtgagagaggggtacaactcacgaatcccttctacagtatctggactacagacattggaacagagctggccatggccttcatcatcgtggctggaatctgcctctgcctctacttcctgtttctatgcttcatggtatttcaggtgtttcggaacatcagtgggaagcagtccagcctgccagctatgagcaaagtccggcggctacactatgaggggctaatttttaggttcaagttcctcatgcttatcaccttggcctgcgctgccatgactgtcatcttcttcatcgttagtcaggtaacggaaggccattggaaatggggcggcgtcacagtccaagtgaacagtgcctttttcacaggcatctatgggatgtggaatctgtatgtctttgctctgatgttcttgtatgcaccatcccataaaaactatggagaagaccagtccaatggcgatctgggtgtccatagtggggaagaactccagctcaccaccactatcacccatgtggacggacccactgagatctacaagttgacccgcaaggaggcccaggagtag

SEQ ID NO: 102 - protein, 507 aa, homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 103 - DNA, 1524 bp, artificial sequence
  cDNA
atggcgggtgcgggcccgaagcggcgcgcgctagcggcgccggcggccgaggagaaggaagaggcgcgggagaagatgctggccgccaagagcgcggacggctcggcgccggcaggcgagggcgagggcgtgaccctgcagcggaacatcacgctgctcaacggcgtggccatcatcgtggggaccattatcggctcgggcatcttcgtgacgcccacgggcgtgctcaaggaggcaggctcgccggggctggcgctggtggtgtgggccgcgtgcggcgtcttctccatcgtgggcgcgctctgctacgcggagctcggcaccaccatctccaaatcgggcggcgactacgcctacatgctggaggtctacggctcgctgcccgccttcctcaagctctggatcgagctgctcatcatccggccttcatcgcagtacatcgtggccctggtcttcgccacctacctgctcaagccgctcttccccacctgcccggtgcccgaggaggcagccaagctcgtggcctgcctctgcgtgctgctgctcacggccgtgaactgctacagcgtgaaggccgccacccgggtccaggatgcctttgccgccgccaagctcctggccctggccctgatcatcctgctgggcttcgtccagatcgggaagggtgatgtgtccaatctagatcccaacttctcatttgaaggcaccaaactggatgtggggaacattgtgctggcattatacagcggcctctttgcctatggaggatggaattacttgaatttcgtcacagaggaaatgatcaacccctacagaaacctgcccctggccatcatcatctccctgcccatcgtgacgctggtgtacgtgctgaccaacctggcctacttcaccaccctgtccaccgagcagatgctgtcgtccgaggccgtggccgtggacttcgggaactatcacctgggcgtcatgtcctggatcatccccgtcttcgtgggcctgtcctgcttcggctccgtcaatgggtccctgttcacatcctccaggctcttcttcgtggggtcccgggaaggccacctgccctccatcctctccatgatccacccacagctcctcacccccgtgccgtccctcgtgttcacgtgtgtgatgacgctgctctacgccttctccaaggacatcttctccgtcatcaacttcttcagcttcttcaactggctctgcgtggccctggccatcatcggcatgatctggctgcgccacagaaagcctgagcttgagcggcccatcaaggtgaacctggccctgcctgtgttcttcatcctggcctgcctcttcctgatcgccgtctccttctggaagacacccgtggagtgtggcatcggcttcaccatcatcctcagcgggctgcccgtctacttcttcggggtctggtggaaaaacaagcccaagtggctcctccagggcatcttctccacgaccgtcctgtgtcagaagctcatgcaggtggtcccccaggagacatag

SEQ ID NO: 104 - protein, 478 aa, homo sapiens
MPPMPSAPPVHPPPDGGWGWIVVGAAFISIGFSYAFPKAVTVFFKEIQQIFHTTYSEIAWISSIMLAVMYAGGPVSSVLVNKYGSRPVVIAGGLLCCLGMVLASFSSSVVQLYLTMGFITGLGLAFNLQPALTIIGKYFYRKRPMANGLAMAGSPVFLSSLAPFNQYLFNTFGWKGSFLILGSLLLNACVAGSLMRPLGPNQTTSKSKNKTGKTEDDSSPKKIKTKKSTWEKVNKYLDFSLFKHRGFLIYLSGNVIMFLGFFAPIIFLAPYAKDQGIDEYSAAFLLSVMAFVDMFARPSVGLIANSKYIRPRIQYFFSFAIMFNGVCHLLCPLAQDYTSLVLYAVFFGLGFGSVSSVLFETLMDLVGAPRFSSAVGLVTIVECGPVLLGPPLAGKLVDLTGEYKYMYMSCGAIVVAASVWLLIGNAINYRLLAKERKEENARQKTRESEPLSKSKHSEDVNVKVSNAQSVTSERETNI

SEQ ID NO: 105 - DNA, 1437 bp, artificial sequence
  cDNA
atgccaccaatgccaagtgccccacctgtgcatccacctccagatggaggatggggttggattgtggttggagcagcttttatctccattggattttcctatgcattccccaaagctgtcaccgtattcttcaaagaaattcagcaaatattccacactacctacagtgaaatagcatggatttcatccattatgctggctgttatgtacgcaggaggtcctgtaagtagtgttttggtgaataaatacggcagccggccggtggtgatagcaggaggcttattatgctgtcttggaatggtgttggcctcctttagtagcagcgtggtacagctgtacctcactatgggattcattacaggtttaggtttagccttcaacctgcaacccgccttaaccataattggcaaatacttctataggaagcgacccatggcaaatggattggccatggcaggaagtcctgttttcttaagttcattggctcctttcaatcagtacctttttaatacttttggctggaaaggaagcttcctgattttgggaagtctacttttgaatgcctgtgtggctggttccctcatgagaccccttggacccaatcaaaccacttctaagtctaaaaataagactggcaaaacagaagatgattcaagcccaaagaaaatcaaaacgaagaaatcaacttgggaaaaagttaataagtatttagatttctccctttttaagcatagaggatttctgatatatctgtctggaaatgtcattatgttcctaggtttttttgcccccattatattcttggctccatatgctaaagaccaaggaattgatgagtactcggcagcttttctgctatctgttatggctttcgttgatatgtttgctaggccttctgtaggattaattgcaaactccaaatatattcgacctcgaattcagtacttcttcagttttgcaatcatgttcaatggagtgtgtcacctcttgtgcccactggcacaggactacacaagcctggtattatatgctgtattttttggccttggatttgggagtgttagcagtgttctctttgaaactctcatggacctcgtgggtgcaccaagattttccagtgccgtcggacttgtcacaattgtggagtgtggcccagttcttcttggccctcctcttgcaggtaaattggtggatttaactggagaatataaatacatgtacatgtcctgtggggctattgtggtagcagcaagcgtgtggctgctcattggcaatgctatcaactatagattgcttgcaaaggaaaggaaggaggaaaatgcaaggcagaagaccagagaatctgaacccttgagcaaatctaaacattcggaagatgttaacgtcaaagtttcaaatgcacagagtgtaacctcagaaagagaaactaacatttaa

(19) United States
(12) Patent Application Publication
(10) Pub. No.: US 2023/0184745 A1
(43) Pub. Date: Jun. 15, 2023

(54) Screening method for effective target - E3 ligase combinations

(71) Applicant: UMC Utrecht Holding B.V., Utrecht (NL)

(72) Inventor: Madelon Maria Maurice, De Bilt (NL)

(73) Assignee: UMC Utrecht Holding B.V., Utrecht (NL)

(21) Appl. No.: 17/926,668

(22) PCT Filed: Jun. 18, 2021
(86) PCT No.: PCT/EP2021/066696

§ 371 Date: Nov. 21, 2022

(30) Foreign Application Priority Data
Mar. 5, 2020 (WO) PCT/EP2021/055551
Jun. 18, 2020 (EP) 20180740.1

(51) Int. Cl. G01N 33/50; C12N 9/10; C07K 14/705; C07K 14/52; C07K 14/71; C07K 16/28; C07K 16/40

(52) U.S. Cl. CPC G01N 33/5035; C12N 9/104; C07K 14/70521; C07K 14/70532; C07K 14/522; C07K 14/71; C07K 16/28; C07K 16/40; C07K 2317/31; C07K 2317/569; G01N 2333/9015; G01N 2440/36; C07K 2319/40

(57) ABSTRACT

The invention pertains to a method to identify an effective combination of a transmembrane E3 ubiquitin ligase and a membrane-bound protein, wherein the combination is effective when the transmembrane E3 ubiquitin ligase is capable of decreasing the surface level of the membrane-bound protein upon forced dimerization, preferably by ubiquitination of the membrane-bound protein. The method of the invention comprises a step of exposing a cell to a heterobifunctional molecule, wherein the heterobifunctional molecule comprises a first binding domain capable of specific binding to an extracellular portion of the transmembrane E3 ubiquitin ligase, and a second binding domain capable of specific binding to an extracellular portion of the membrane-bound protein. The method further comprises a step of determining the decrease in surface level of the membrane-bound protein. The invention additionally pertains to a heterobifunctional molecule targeting an effective combination of a transmembrane E3 ubiquitin ligase and a membrane-bound protein.

### FIELD OF THE INVENTION

The invention pertains to the field of molecular cell biology, in particular to the fields of targeted molecular therapy and cancer cell biology. The invention pertains to a method for screening effective combinations of a target membrane-bound protein and an E3 ubiquitin ligase, and the generation of heterobifunctional molecules simultaneously targeting these discovered effective combinations. Hence, the invention further pertains to the use of heterobifunctional molecules that can simultaneously bind to a transmembrane ubiquitin ligase and a membrane-bound protein, to mediate internalisation of the membrane-bound protein.

### BACKGROUND

Cells communicate with their environment by the activity of plasma membrane-embedded receptors that capture external chemical signals and initiate an intracellular signaling cascade to drive a cellular response. Receptor availability at the cell surface is a critical determinant of signal specificity and sensitivity and misregulation of these events is frequently linked to the development or progression of a disease, such as, but not limited to, cancer, auto-immune diseases, neurological disorders and inflammatory disorders, as well as therapy resistance.

For example, mutational activation or overexpression of receptors is a major and widely recognized cancer-promoting mechanism in multiple tissues (e.g. EGFR, ERBB2, PDGFR, TGFβR, IGFR1, GHR, FZD, LRP6). The dependency of cancer cells on aberrant receptor activity has instigated the development of various neutralizing antibodies and small molecule inhibitors. Successful neutralization of receptor activity however requires the generation of potent binders that reach sufficient plasma concentrations to display high efficacy without inducing toxicity, which may prove difficult in case of non-covalent interactors. Furthermore, compensatory receptor stabilization or upregulation is a major pathway of resistance.

Posttranslational modification of the cytosolic regions of membrane-bound receptors with ubiquitin drives their rapid removal from the cell surface via induced endocytosis. The internalised receptors may subsequently be subjected to lysosomal degradation. In healthy stem cells, high levels of Wnt signalling drive the expression of two homologous membrane-bound ubiquitin ligases, RNF43 and ZNRF3, that are known to mediate ubiquitination and removal of Frizzled (FZD), the receptors for Wnt, from the cell surface (Koo et al, Nature 2012, 488(7413):665-9). This negative feedback loop thus serves to regulate the sensitivity of stem cells to Wnt by controlling the effective number of Frizzled (FZD) receptors on the cell surface. The activity of RNF43/ZNRF3 towards FZD is neutralized in the stem cell niche by the secreted protein R-spondin (RSPO) that forms a complex with LGR4/5 receptors as well as RNF43/ZNRF3 (Hao et al, Nature 2012, 485(7397):195-200). Next, this trimeric RSPO-LGR4/5-RNF43/ZNRF3 complex undergoes removal from the cell surface, leading to stabilization of FZD receptor expression and increased levels of Wnt signaling. Wnt signaling is frequently misregulated in cancer. Such cancers display increased expression of Wnt target genes, including RNF43 and ZNRF3.

E3 ubiquitin ligases recruit an E2 ubiquitin-conjugating enzyme that has been loaded with ubiquitin to a protein substrate and assists or directly catalyses the transfer of ubiquitin to the protein substrate.

Ubiquitination of receptors mediated by transmembrane ubiquitin E3 ligases is known to result in endocytosis and subsequent breakdown of the ubiquitinated substrate. It is known in the art that such breakdown preferably takes place in the lysosome. Lysosomal degradation requires ligation of monoubiquitin, multiubiquitin, Lys11-, Lys29-, Lys48- or Lys63-linked poly-ubiquitin chains to membrane-bound receptors. This is in contrast to the activity of cytosolic ubiquitin ligases, which mainly employ the proteasomal degradation pathway, i.e. by the coupling of Lys11-, Lys29- or Lys48-linked poly-ubiquitin chains to cytosolic target proteins.

Hence, transmembrane E3 ubiquitin ligases may interact with different members of the E2 enzyme family to selectively target membrane-bound substrates. The ubiquitinated substrate will be internalised and may subsequently be degraded, preferably via lysosomal degradation.

There is still a strong need in the art to effectively target and inhibit activity of membrane-bound receptors, especially membrane-bound receptors that are involved in the development or progression of a disease. There is in particular a strong need in the art to effectively target and inhibit the activity of membrane-bound receptors that are involved in the development or progression of e.g. cancer, auto-immune diseases, neurological disorders, rare diseases and inflammatory disorders, as well as therapy resistance.

### SUMMARY OF THE INVENTION

The invention is summarized in the following embodiments:

Embodiment 1. A method for identifying an effective combination of a transmembrane E3 ubiquitin ligase and a membrane-bound protein, wherein the combination is effective when the transmembrane E3 ubiquitin ligase is capable of decreasing the surface level of the membrane-bound protein upon simultaneous binding to a heterobifunctional molecule, preferably by ubiquitination of the membrane-bound protein, and wherein the method comprises the steps of:
a) Providing a cell, wherein the cell expresses the transmembrane E3 ubiquitin ligase and the membrane-bound protein at its cell surface;
b) Exposing the cell to the heterobifunctional molecule, wherein the heterobifunctional molecule comprises:
   i) a first binding domain capable of specific binding to an extracellular portion of the transmembrane E3 ubiquitin ligase; and
   ii) a second binding domain capable of specific binding to an extracellular portion of the membrane-bound protein; and
c) determining the surface level of the membrane-bound protein of the cell,
wherein a decrease in the surface level of the membrane-bound protein indicates that the combination is an effective combination, and wherein the decrease is preferably a decrease as compared to the surface level of the membrane-bound protein of the cell prior to step b).

Embodiment 2. A method according to embodiment 1, wherein the membrane-bound protein is a transmembrane protein.

Embodiment 3. A method according to embodiment 1 or 2, wherein the transmembrane E3 ubiquitin ligase is selected from the group consisting RNF43, RNF167, ZNRF3, RNF13, AMFR, MARCH1, MARCH2, MARCH4, MARCH8, MARCH9, RNF149, RNF145, RNFT1, RNF130 and RNF128.

Embodiment 4. A method according to any one of the preceding embodiments, wherein at least one of:
the transmembrane E3 ubiquitin ligase comprises a first extracellular non-native epitope tag, and wherein the first binding domain of the heterobifunctional molecule binds to the first non-native epitope tag; and
the membrane-bound protein comprises a second extracellular non-native epitope tag, and wherein the second binding domain of the heterobifunctional molecule binds to the second non-native epitope tag.

Embodiment 5. A method according to embodiment 4, wherein the first and second non-native epitope tags are different tags.

Embodiment 6. A method according to embodiment 4 or 5, wherein the first non-native epitope tag is at least one of an alpha tag and an E6 tag, and/or wherein the second non-native epitope tag is at least one of an alpha tag and an E6 tag.

Embodiment 7. A method according to any one of embodiments 4-6, wherein at least one of the first and second non-native epitope tag is located in at least one of
i) the N-terminus;
ii) the C-terminus; and/or
iii) an extracellular loop region, of respectively the transmembrane E3 ubiquitin ligase and the membrane-bound protein.

Embodiment 8. A method according to any one of the preceding embodiments, wherein the heterobifunctional molecule is a bi-specific antibody, preferably a bi-specific nanobody.

Embodiment 9. A method according to embodiment 8, wherein the first binding domain of the heterobifunctional molecule is an anti-Alpha VHH and the second binding domain is an anti-E6 VHH, or wherein the first binding domain of the heterobifunctional molecule is an anti-E6 VHH and the second binding domain is an anti-Alpha VHH.

Embodiment 10. A method according to any one of the preceding embodiments, wherein the membrane-bound protein comprises a third non-native epitope tag and/or wherein the transmembrane ubiquitin E3 ligase comprises a fourth non-native epitope tag, preferably wherein the third and/or fourth epitope tag is at least one of a His-tag, FLAG-tag, and a myc-tag.

Embodiment 11. A method according to any one of the preceding embodiments, wherein the cell surface levels of the membrane-bound protein in step c) are determined by detecting the protein on the cell surface, preferably by immunofluorescence.

Embodiment 12. A method according to any one of the preceding embodiments, wherein the combination is effective when the cell surface levels of the membrane-bound protein are decreased at least about 10%, 20%, 30%, 40%, 50%, 60%, 70%, 80%, 90% or at least about 95% as compared to the cell surface levels of the membrane-bound protein prior to step b), preferably at least about 60%, 70%, 80%, 90% or at least about 95% as compared to the cell surface levels of the membrane-bound protein prior to step b).

Embodiment 13. A method according to any one of embodiments 4-11, wherein in step a) a first and a second cell is provided, wherein
the first cell expresses a first transmembrane E3 ubiquitin ligase and a first membrane-bound protein at its cell surface; and
the second cell expresses a second transmembrane E3 ubiquitin ligase and the first membrane-bound protein and its cell surface,
wherein the first and second transmembrane E3 ubiquitin ligase are different ligases comprising the same first extracellular non-native epitope tag;
wherein in step b) the first and the second cell is exposed the heterobifunctional molecule,
wherein the heterobifunctional molecule comprises:
   i) a first binding domain capable of specific binding to the first non-native epitope tag; and
   ii) a second binding domain capable of specific binding to an extracellular portion of the membrane-bound protein, preferably to the second non-native epitope tag; and
wherein in step c) the surface level of the membrane-bound protein of the first and second cell are determined, and wherein a combination is effective when the cell surface levels of the membrane-bound protein in the first cell are decreased at least about 10%, 20%, 30%, 40%, 50% or at least about 60% as compared to the cell surface levels of the membrane-bound protein in the second cell after step b).

Embodiment 14. A method according to embodiment 13, wherein a third, fourth or further cells are provided expressing respectively a third, a fourth or a further transmembrane E3 ubiquitin ligase and the first membrane-bound protein at their cell surface,
wherein the transmembrane E3 ubiquitin ligases are different ligases comprising the same first extracellular non-native epitope tag,
and wherein the combination is effective when the cell surface levels of the membrane-bound protein in the first cell are decreased at least about 5%, 10%, 20%, 30%, 40%, 50%, 60%, 70%, 80%, 90% or at least about 95% as compared to the cell surface levels of the membrane-bound protein in the second, third, fourth and further cells after step b),
and/or wherein the method is performed in a multiplexed manner.

Embodiment 15. A method according to any one of the preceding embodiments, wherein the decrease in the surface level of the membrane-bound protein is determined by a decrease in the total amount of the membrane-bound protein in the cell, preferably as determined by microscopy, biochemical analysis and/or FACS.

Embodiment 16. A method according to any one of the preceding embodiments, wherein the cell provided in step a) overexpresses, optionally permanently overexpresses, at least one of the transmembrane E3 ubiquitin ligase and the membrane-bound protein.

Embodiment 17. A method according to any one of the preceding embodiments, wherein the cell provided in step a) expresses the transmembrane E3 ubiquitin ligase and the membrane-bound protein at endogenous levels.

Embodiment 18. A method according to embodiment 17, wherein in the cell provided in step a) a genomic sequence encoding the transmembrane E3 ubiquitin ligase has been modified to incorporate a sequence encoding the first, and optional fourth, non-native epitope tag.

Embodiment 19. A method according to embodiment 17 or 18, wherein in the cell provided in step a) a genomic sequence encoding the membrane-bound protein has been modified to incorporate a sequence encoding the second, and optional third, non-native epitope tag.

Embodiment 20. A method according to any one of the preceding embodiments, wherein the heterobifunctional molecule comprises a peptide linker between the first binding domain and the second binding domain, and wherein preferably the peptide linker is (GGGGS)n, wherein n is preferably 1, 2, 3, 4, 5, 6, 7, 8, 9, 10, 11, 12, 13, 14 or 15, preferably wherein n is 3 or 5.

Embodiment 21. A heterobifunctional molecule comprising a first and a second binding domain, wherein
i) the first binding domain is capable of specific binding to a transmembrane E3 ubiquitin ligase; and
ii) the second binding domain is capable of specific binding to a membrane-bound protein,
and wherein the transmembrane E3 ligase and the membrane-bound protein are an effective combination as determined in the method of any one of embodiments 1-20.

Embodiment 22. A heterobifunctional molecule according to embodiment 21, wherein the molecule binds an extracellular portion of the transmembrane E3 ubiquitin ligase and an extracellular portion of the membrane-bound protein.

Embodiment 23. A heterobifunctional molecule according to embodiment 21 or 22, wherein the membrane-bound protein is a receptor, preferably a receptor involved in at least one of cancer, an auto-immune disease, a neurological disorder and an inflammatory disorder.

Embodiment 24. A heterobifunctional molecule according to any one of embodiments 21-23, wherein the heterobifunctional molecule is a bi-specific antibody, preferably a bi-specific nanobody.

Embodiment 25. A heterobifunctional molecule according to any one of embodiments 21-24 for use as a medicament.

### Definitions

Various terms relating to the methods, compositions, formulations, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. The indefinite article “a” or “an” thus usually means “at least one”. Thus, for example, reference to “a cell” includes a combination of two or more cells, and the like.

“About” and “approximately”: these terms, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: The term “and/or” refers to a situation wherein one or more of the stated cases may occur, alone or in combination with at least one of the stated cases, up to with all of the stated cases.

“Comprising”: this term is construed as being inclusive and open ended, and not exclusive. Specifically, the term and variations thereof mean the specified features, steps or components are included. These terms are not to be interpreted to exclude the presence of other features, steps or components.

Exemplary”: this terms means “serving as an example, instance, or illustration,” and should not be construed as excluding other configurations disclosed herein.

The term “heterobifunctional molecule” is defined herein as a molecule comprising two different functional binding domains. In particular, the heterobifunctional molecule of the invention has a first functional binding domain for binding a transmembrane E3 ubiquitin ligase and a separate second functional binding domain for binding a second molecule. As the name “heterobifunctional” already indicates, the second functional binding domain binds a second molecule, wherein the second molecule is not the same molecule, i.e. not the same transmembrane E3 ubiquitin ligase, that can be bound by the first functional binding domain. Preferably, the second functional binding domain does not bind to a transmembrane E3 ubiquitin ligase.

The term “protein” or “polypeptide” refers to a molecule consisting of a chain of amino acids, without reference to a specific mode of action, size, 3 dimensional structure or origin. A “fragment” or “portion” of a protein may thus still be referred to as a “protein.” A protein as defined herein and as used in any method as defined herein may be an isolated protein. An “isolated protein” is used to refer to a protein which is no longer in its natural environment, for example in vitro or in a recombinant bacterial or plant host cell. Preferably, the protein comprises more than 50 amino acid residues.

The term “proteinaceous molecule” is herein understood as a molecule comprising a short chain of amino acid monomers linked by peptide (amide) bonds. The short chain of amino acid monomers comprise 2 or more amino acid residues. Preferably, the chain of amino acids has at least 2, 3, 4, 5, 6, 7, 8, 9, 10, 11, 12, 13, 14, 15 amino acid residues. Preferably, the there are no more than 100 amino acid residues. Preferably, there are no more than 50 amino acid residues in the proteinaceous molecule. Preferably, the proteinaceous molecule has about 2-100, 3-50, 4-40 or 5-30, or 6-20 amino acid residues. Preferably, the proteinaceous molecule has 2, 3, 4, 5, 6, 7, 8, 9, 10, 11, 12, 13, 14 or 15 amino acid residues. Optionally, the proteinaceous molecule comprises one or more additional organic moieties, such as, but not limited to a linking moiety to generate a cyclised proteinaceous molecule.

An “aptamer” preferably is a nucleic acid molecule having a particular nucleotide sequence. An aptamer can include any suitable number of nucleotides. An aptamer may comprise RNA or DNA, or comprises both ribonucleotide residues and deoxyribonucleotide residues. An aptamer may be single stranded, double stranded, or contain double stranded or triple stranded regions. In addition, an aptamer may comprise chemical modified residues, e.g. to improve its stability.

An aptamer will typically be between about 10 and about 300 nucleotides in length. More commonly, an aptamer will be between about 30 and about 100 nucleotides in length.

Aptamers to a given target (i.e. a transmembrane E3 ubiquitin ligase or a further transmembrane protein) include nucleic acids that may be identified from a candidate mixture of nucleic acids using a method comprising the steps of: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield an enriched mixture of nucleic acids, whereby aptamers of the target molecule are identified.

It is recognized that affinity interactions are a matter of degree; however, in this context, the “specific binding affinity” of an aptamer for its target means that the aptamer binds to its target generally with a much higher degree of affinity than it binds to other, non-target, components in a mixture or sample.

Aptamers have specific binding regions which are capable of forming complexes with an intended target molecule in an environment wherein other substances in the same environment are not complexed to the nucleic acid. The specificity of the binding can be defined in terms of the comparative dissociation constants (Kd) of the aptamer for its ligand as compared to the dissociation constant of the aptamer for other materials in the environment or unrelated molecules in general. Typically, the Kd for the aptamer with respect to its ligand will be at least about 10-fold less than the Kd for the aptamer with unrelated material or accompanying material in the environment. Even more preferably, the Kd will be at least about 50-fold less, more preferably at least about 100-fold less, and most preferably at least about 200-fold less.

In certain embodiments, an aptamer that binds to the transmembrane protein has a dissociation constant (K_{d}) of ≤1 mM, ≤100 nM, ≤10 nM, ≤1 nM, or ≤0.1 nM. In certain embodiments, the anti-transmembrane protein antibody binds to an epitope that is conserved among different species.

In certain embodiments, an aptamer that binds to the transmembrane E3 ubiquitin ligase has a dissociation constant (K_{d}) of 1 mM, 100 nM, 10 nM, 1 nM, or 0.1 nM. In certain embodiments, the anti-transmembrane protein antibody binds to an epitope that is conserved among different species.

The term “antibody” is used in the broadest sense and specifically covers, e.g. monoclonal antibodies, including agonists and antagonist, neutralizing antibodies, full length or intact monoclonal antibodies, polyclonal antibodies, multivalent antibodies, single chain antibodies and functional fragments of antibodies, including Fab, Fab′, F(ab′)2 and Fv fragments, diabodies, triabodies, single domain antibodies (sdAbs), heavy-chain antibodies, nanobodies, as long as they exhibit the desired biological and/or immunological activity.

The term “immunoglobulin” (Ig) is used interchangeable with antibody herein. An antibody can be human and/or humanized.

The term “anti-transmembrane E3 ubiquitin ligase antibody” specifically covers, e.g. single anti-transmembrane E3 ubiquitin ligase monoclonal antibodies, including agonists and antagonist, preferably agonists, neutralizing antibodies, full length or intact monoclonal antibodies, polyclonal antibodies, naked antibodies, multivalent antibodies, single chain anti-transmembrane E3 ubiquitin ligase antibodies and fragments of anti-transmembrane E3 ubiquitin ligase antibodies, including Fab, Fab′, F(ab′)2 and Fv fragments, diabodies, triabodies, single domain antibodies (sdAbs), heavy-chain antibodies and nanobodies, as long as they exhibit the desired biological and/or immunological activity. A preferred antibody can be a nanobody. Preferably, the anti-transmembrane E3 ubiquitin ligase antibody binds specifically to an E3 ubiquitin ligase as defined herein below.

The term “anti-transmembrane protein antibody” specifically covers, e.g. single anti-transmembrane protein monoclonal antibodies, including agonists and antagonist, preferably antagonists, neutralizing antibodies, full length or intact monoclonal antibodies, polyclonal antibodies, naked antibodies, multivalent antibodies, single chain anti-transmembrane protein antibodies and fragments of anti-transmembrane protein antibodies, including Fab, Fab′, F(ab′)2 and Fv fragments, diabodies, triabodies, single domain antibodies (sdAbs), heavy-chain antibodies and nanobodies, as long as they exhibit the desired biological and/or immunological activity. A preferred antibody can be a nanobody. Preferably, the anti-transmembrane protein antibody binds specifically to a transmembrane protein as defined herein below.

The term “anti-transmembrane E3 ubiquitin ligase antibody” or “an antibody that binds to a transmembrane E3 ubiquitin ligase” refers to an antibody that is capable of binding an transmembrane E3 ubiquitin ligase with sufficient affinity such that the antibody is useful as a first binding domain of a heterobifunctional molecule as defined herein. Preferably, the extent of binding of an anti-transmembrane E3 ubiquitin ligase antibody to an unrelated protein is less than about 10% of the binding of the antibody to the transmembrane E3 ubiquitin ligase as measured, e.g., by a radioimmunoassay (RIA) or ELISA. In certain embodiments, an antibody that binds to the transmembrane E≤3 ubiquitin ligase has a dissociation constant (K_{d}) of ≤1 mM, ≤100 nM, ≤10 nM, ≤1 nM, or ≤0.1 nM. In certain embodiments, the anti-transmembrane E3 ubiquitin ligase antibody binds to an epitope that is conserved among different species.

The term “anti-transmembrane protein antibody” or “an antibody that binds to a transmembrane protein” refers to an antibody that is capable of binding a specific or selected transmembrane protein with sufficient affinity such that the antibody is useful as a second binding domain of a heterobifunctional molecule as defined herein. Preferably, the extent of binding of an anti-transmembrane protein antibody to an unrelated protein is less than about 10% of the binding of the antibody to the transmembrane protein as measured, e.g., by a radioimmunoassay (RIA) or ELISA. In certain embodiments, an antibody that binds to the transmembrane protein has a dissociation constant (K_{d}) of ≤1 mM, ≤100 nM, ≤10 nM, ≤1 nM, or ≤0.1 nM. In certain embodiments, the anti-transmembrane protein antibody binds to an epitope that is conserved among different species.

An antibody “which binds” an antigen of interest, i.e. the transmembrane E3 ubiquitin ligase or a further transmembrane protein of interest, is one that binds said antigen with sufficient affinity such that the antibody is useful as respectively a first binding domain or second binding domain of a heterobifunctional molecule as defined herein.

The antibody acting as a first binding domain or as a second binding domain in the heterobifunctional molecule can be a basic 4-chain antibody. Such basic 4-chain antibody unit is preferably a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain).

In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for p and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}”. These domains are generally the most variable parts of an antibody and contain the antigen-binding sites.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” (HVRs) that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

An “intact” antibody is one which comprises an antigen-binding site as well as a CL and at least heavy chain constant domains, C_{H}1, C_{H}2 and C_{H}3. The constant domains may be native sequence constant domains (e.g. human native sequence constant domains) or amino acid sequence variants thereof.

“Antibody fragments” comprise a portion of an intact antibody, preferably at least the antigen binding and/or variable region of the intact antibody. Examples of antibody fragments include Fab, Fab′, F(ab′)₂, and Fv fragments; diabodies; triabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments. In one embodiment, an antibody fragment comprises an antigen binding site of the intact antibody and thus retains the ability to bind the antigen.

The term “nanobody” is well-known in the art. A nanobody is an antibody fragment comprising or consisting of a VHH domain of a heavy chain only antibody. The terms “nanobody”, “single-domain antibody” and “single-domain antibody fragment” may be used interchangeable herein. The single-domain antibody fragment has a single monomeric variable antibody domain, and preferably a molecular weight of about 12-15 kDa. A nanobody, like a whole antibody, is able to selectively bind a specific antigen. A nanobody may be derivable from dromedaries, camels, llamas, alpacas or sharks. A preferred nanobody is derivable from the camelidae family, preferably derivable from a Llama.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site.

Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy-terminus of the C_{H}1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments, which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen recognition and binding site. This fragment consists of a dimer of one heavy- and one light-chain variable region domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site, in contrast to polyclonal antibody preparations which include different antibodies directed against different determinants (epitopes). Monoclonal antibodies are advantageous in that they may be synthesized uncontaminated by other antibodies. The modifier “monoclonal” is not to be construed as requiring production of the antibody by any particular method. For example, the monoclonal antibodies useful as a first or second binding domain in the heterobifunctional molecule of the invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc.), and human constant region sequences.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, a few framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma and Immunol., 1:105-115 (1998); Harris, Biochem. Soc. Transactions, 23:1035-1038 (1995); Hurle and Gross, Curr. Op. Biotech., 5:428-433 (1994).

The term “hypervariable region”, “HVR”, when used herein refers to the regions of an antibody variable domain which are hypervariable in sequence and/or form structurally defined loops that are responsible for antigen binding. Generally, antibodies comprise six hypervariable regions; three in the VH (H1, H2, H3), and three in the VL (L1, L2, L3). A number of hypervariable region delineations are in use and are encompassed herein. The hypervariable regions generally comprise amino acid residues from a “complementarity determining region” or “CDR” (e.g., around about residues 24-34 (L1), 50-56 (L2) and 89-97 (L3) in the VL, and around about 31-35 (H1), 50-65 (H2) and 95-102 (H3) in the VH when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the VL, and 26-32 (H1), 52-56 (H2) and 95-101 (H3) in the VH when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the VL, and 27-38 (H1), 56-65 (H2) and 105-120 (H3) in the VH when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the VL, and 28, 36 (H1), 63, 74-75 (H2) and 123 (H3) in the VH when numbered in accordance with Honneger, A. and Plunkthun, A. J. (Mol. Biol. 309:657-670 (2001)). The hypervariable regions/CDRs of the antibodies of the invention are preferably defined and numbered in accordance with the IMGT numbering system.

“Framework” or “FR” residues are those variable domain residues other than the hypervariable region residues herein defined.

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody which mimics at least one of the functional activities of a polypeptide of interest.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

A “K_{d}” or “K_{d} value” can be measured by using surface plasmon resonance assays using a BIAcore™-2000 or a BIAcore™-3000 (BIAcore, Inc., Piscataway, N.J.) at 25° C. with immobilized antigen CM5 chips at ˜10-50 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 μg/ml (˜0.2 μM) before injection at a flow rate of 5 μl/minute to achieve approximately 10 response units (RU) of coupled protein. Following the injection of antigen, 1M ethanolamine is injected to block unreacted groups. For kinetics measurements, two-fold serial dilutions of the antibody or Fab (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (K_{d}) is calculated as the ratio k_{off}/kₒₙ. See, e.g., Chen, Y., et al., (1999) J. Mol Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stir red cuvette.

An “on-rate” or “rate of association” or “association rate” or “kₒₙ” according to this invention can also be determined with the same surface plasmon resonance technique described above using a BIAcore™-2000 or a BIAcore™-3000 (BIAcore, Inc., Piscataway, N.J.) as described above.

Preferably, the antibody for use in the heterobifunctional molecule as a first or second binding domain does not significantly cross-react with other proteins.

The term “antigen-binding protein” and “binding domain” of the heterobifunctional molecule of the invention may be used interchangeably herein.

The term “epitope” is the portion of a molecule that is bound by respectively the first or second binding domain of the heterobifunctional molecule of the invention. The term includes any determinant capable of specifically binding to an antigen binding protein, e.g. specifically binding to a first or second domain of a heterobifunctional molecule as defined herein below. An epitope can be contiguous or non-contiguous (e.g., in a polypeptide, amino acid residues that are not contiguous to one another in the polypeptide sequence but that within in context of the molecule are bound by the antigen binding protein). Epitopes preferably reside on a transmembrane E3 ubiquitin ligase as defined herein or on a further transmembrane protein of interest as defined herein.

Epitope determinants may include chemically active surface groupings of molecules such as amino acids, sugar side chains, phosphoryl, sulfonyl or sulfate groups, and may have specific three dimensional structural characteristics, and/or specific charge characteristics. Generally, antibodies specific for a particular target antigen will preferentially recognize an epitope on the target antigen in a complex mixture of proteins and/or macromolecules.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody.

The term “Fc region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, a heterobifunctional molecule comprising an antibody having an Fc region according to this invention can comprise an antibody with K447 or with K447 removed.

“Amino acid sequence”: This refers to the order of amino acid residues of, or within a protein. In other words, any order of amino acids in a protein may be referred to as amino acid sequence.

“Nucleotide sequence”: This refers to the order of nucleotides of, or within a nucleic acid. In other words, any order of nucleotides in a nucleic acid may be referred to as nucleotide sequence.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide.

The term “complementarity” is herein defined as the sequence identity of a nucleotide sequence to a fully complementary strand (e.g. the second, or reverse, strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, word length=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the terms “prevent”, “preventing”, and “prevention” refer to the prevention or reduction of the recurrence, onset, development or progression of a disease, preferably a disease as defined herein below, or the prevention or reduction of the severity and/or duration of the disease or one or more symptoms thereof.

As used herein, the terms “therapies” and “therapy” can refer to any protocol(s), method(s) and/or agent(s) that can be used in the prevention, treatment, management or amelioration of the disease, preferably a disease as defined herein below, or one or more symptoms thereof.

As used herein, the terms “treat”, “treating” and “treatment” refer to the reduction or amelioration of the progression, severity, and/or duration of a disease, preferably a disease as defined herein below, and/or reduces or ameliorates one or more symptoms of the disease.

As used herein, the term “effective amount” refers to the amount of a therapy, e.g., a prophylactic or therapeutic agent, preferably a heterobifunctional molecule as defined herein, which is sufficient to reduce the severity, and/or duration of a disease, ameliorate one or more symptoms thereof, prevent the advancement of the disease, or cause regression of the disease, or which is sufficient to result in the prevention of the development, recurrence, onset, or progression of the disease or one or more symptoms thereof, or enhance or improve the prophylactic and/or therapeutic effect(s) of another therapy (e.g., another therapeutic agent). Preferably, the disease is a disease as defined herein below.

### DETAILED DESCRIPTION

The current invention concerns the inventive concept to employ heterobifunctional molecules for targeted internalisation and subsequent degradation of membrane-bound proteins. The heterobifunctional molecules of the invention can simultaneously bind a transmembrane ubiquitin ligase and a membrane-bound protein, such as a cancer-promoting receptor. Induced proximity (i.e. “forced dimerization”) of the ubiquitin ligase with the desired target transmembrane protein will result in ubiquitination of the target followed by its removal from the cell surface and subsequent degradation. E.g. as a consequence, cancer cell growth is compromised. A schematic representation of an exemplary embodiment of the invention is provided in FIG. 1.

The advantages of this approach include at least the following:
i) The heterobifunctional molecules of the invention allow for strong gains in potency, requiring only sub-stoichiometric amounts of the molecule compared to their target molecules when compared to conventional ‘occupancy-based’ therapeutics.
ii) The required specific binding of two proteins, i.e. a transmembrane E3 ubiquitin ligase as well as a membrane-bound protein also reduces potential off-target toxicity. Preferably, ubiquitin ligases that localize to the plasma membrane and display increased expression in cancer cells will be employed.
iii) Targeting protein degradation leads to a prolonged pharmacodynamic effect, due to the time required to synthesize sufficient amounts of a new transmembrane protein.

iv) The heterobifunctional molecules bind to the extracellular protein parts and thus do not need to cross the cell membrane.
v) Cancer cells are known to abundantly express several types of transmembrane E3 ubiquitin ligases, such as RNF43 and ZNRF3 in cancer cells with self-renewing properties. In this case 4 alleles generate proteins that perform ubiquitination activity, lowering the chances for mutational inactivation and resistance.
   The inventors further discovered that not all membrane-bound proteins can be effectively targeted by any transmembrane E3 ubiquitin ligase, i.e. bringing a membrane-bound protein in close proximity of a transmembrane E3 ubiquitin ligase does not necessarily result in cell surface removal of the membrane-bound protein. Thus for the development of effective heterobifunctional molecules, a screening method should be employed to determine those combinations that result in effective internalisation of the membrane-bound protein when brought in close proximity of a transmembrane E3 ubiquitin ligase.

The inventors discovered an efficient method to screen for effective combinations of a transmembrane E3 ubiquitin ligase and a membrane-bound protein, e.g. combinations wherein the induced proximity (“forced dimerization”) of the transmembrane ubiquitin E3 ligase and the membrane-bound protein results in cell surface removal of the membrane-bound protein. Using this straightforward method, effective heterobifunctional molecules can be constructed, targeting the effective combination of the transmembrane E3 ubiquitin ligase and a membrane-bound protein.

A particular advantage of the method described herein is that a single heterobifunctional molecule can be used to screen different combinations of a transmembrane E3 ubiquitin ligase and a membrane-bound protein, e.g. by using the same first epitope for all transmembrane E3 ubiquitin ligases and the same second epitope for all membrane-bound proteins. This provides for an objective method for determining effective combinations, without having to take into account any variabilities that may exist between different heterobifunctional molecules, such as a variable binding affinity.

Hence in an aspect, the invention pertains to a heterobifunctional molecule comprising a first and a second binding domain. The first binding domain is capable of specific binding to a transmembrane E3 ubiquitin ligase and the second binding domain is capable of binding to a specific membrane-bound protein. The combination of the transmembrane E3 ubiquitin ligase and the membrane-bound protein is preferably identified using a screening method as described herein.

Simultaneous binding of the transmembrane E3 ubiquitin ligase and the membrane-bound protein brings these two molecules in close proximity of each other. As a result, the transmembrane E3 ubiquitin ligase can subsequently ubiquitinate the membrane-bound protein.

Therefore preferably, simultaneous binding of the heterobifunctional molecule to the transmembrane E3 ubiquitin ligase and the membrane-bound protein results in ubiquitination of the membrane-bound protein.

Ubiquitination is known to result in degradation of the ubiquitinated protein. Therefore in addition, preferably simultaneous binding of the heterobifunctional molecule to the transmembrane E3 ubiquitin ligase and the membrane-bound protein results in degradation of the membrane-bound protein.

Simultaneous binding of the transmembrane E3 ubiquitin ligase and the membrane-bound protein brings these two molecules in close proximity of each other. As a result, the membrane-bound protein may be internalized and preferably subsequently degraded.

### Screening Method

Prior to the manufacture of a heterobifunctional molecule binding to native epitopes, such as a heterobifunctional molecule as defined herein for use in the treatment of a disease, first an effective combination of a transmembrane E3 ubiquitin ligase and a membrane-bound protein may be identified. Preferably the combination is considered an effective combination when the transmembrane E3 ubiquitin ligase is capable of decreasing the surface level of the membrane-bound protein, preferably by ubiquitination of the membrane-bound protein, preferably when the E3 ligase and the membrane-bound protein are brought in close proximity of each other, i.e. there is preferably a forced dimerization of the transmembrane E3 ubiquitin ligase and the membrane-bound protein. Hence preferably, the combination is considered an effective combination when the transmembrane E3 ubiquitin ligase is capable of decreasing the surface level of the membrane-bound protein, preferably by ubiquitination of the membrane-bound protein, upon forced dimerization of the transmembrane E3 ubiquitin ligase and the membrane-bound protein. Preferably, the transmembrane E3 ubiquitin ligase and the membrane-bound protein are brought in close proximity by simultaneous binding to a heterobifunctional molecule as defined herein. Hence preferably, the combination is considered an effective combination when the transmembrane E3 ubiquitin ligase is capable of decreasing the surface level of the membrane-bound protein upon simultaneous binding of the transmembrane E3 ubiquitin ligase and the membrane-bound protein to a heterobifunctional molecule, preferably a heterobifunctional molecule as defined herein.

The inventors have developed a method for effective screening for suitable combinations of a transmembrane E3 ubiquitin ligase and a membrane-bound protein, e.g. combinations that can be effectively targeted by a heterobifunctional molecule as defined herein.

In an aspect, the invention therefore pertains to a method for identifying an effective combination of a transmembrane E3 ubiquitin ligase and a membrane-bound protein, wherein a combination is an effective combination when the transmembrane E3 ubiquitin ligase is capable of decreasing the surface level of the membrane-bound protein. Preferably, the combination is an effective combination when the transmembrane E3 ubiquitin ligase can decrease the surface level of the membrane-bound protein by ubiquitination of the membrane-bound protein, preferably followed by internalisation of the ubiquitinated membrane-bound protein. The internalised ubiquitinated membrane-bound protein may subsequently be degraded, preferably in the lysosome. Preferably the method comprises the steps of:
a) Providing a cell, wherein the cell expresses the transmembrane E3 ubiquitin ligase and the membrane-bound protein at its cell surface;
b) Exposing the cell to a heterobifunctional molecule, wherein the heterobifunctional molecule comprises:
   i) a first binding domain capable of specific binding to an extracellular portion of the transmembrane E3 ubiquitin ligase; and
   ii) a second binding domain capable of specific binding to an extracellular portion of the membrane-bound protein; and
c) determining the surface level of the membrane-bound protein of the cell, wherein a decrease in the surface level of the membrane-bound protein indicates that the combination is an effective combination. Preferably, the decrease is a decrease as compared to the surface level of the membrane-bound protein of the cell prior to step b). Preferably the decrease in protein levels is a decrease as compared to the protein levels of the membrane-bound protein of a same or similar cell that is not exposed to the heterobifunctional molecule, e.g. as compared to the protein levels of the membrane-bound protein in the cell provided in step a) of the method of the invention.

The invention further pertains to a method for decreasing the surface level of a membrane-bound protein of a cell. The method preferably comprises the steps of
a) Providing the cell, wherein the cell expresses a transmembrane E3 ubiquitin ligase and the membrane-bound protein at its cell surface; and
b) Exposing the cell to a heterobifunctional molecule as defined herein. The heterobifunctional molecule comprises:
   i) a first binding domain capable of specific binding to an extracellular portion of the transmembrane E3 ubiquitin ligase; and
   ii) a second binding domain capable of specific binding to an extracellular portion of the membrane-bound protein.

The method preferably further comprises a step c) of determining the surface levels of the membrane-bound protein of the cell. The decrease is preferably a decrease as compared to the surface levels of the membrane-bound protein of the cell prior to step b). Preferably the decrease in protein levels is a decrease as compared to the protein levels of the membrane-bound protein of a same or similar cell that is not exposed to the heterobifunctional molecule, e.g. as compared to the protein levels of the membrane-bound protein in the cell provided in step a) of the method of the invention.

The method is preferably an ex vivo method, preferably an in vitro method.

### Step a): Providing a Cell

In step a) of the method of the invention a cell is provided. Any suitable cell for expression of the transmembrane E3 ubiquitin ligase and the membrane-bound protein may be used in the method of the invention. The cell preferably expresses the transmembrane E3 ubiquitin ligase and the membrane-bound protein at its cell surface. Preferably, the cell is an immortalized cell, preferably a cell line, preferably a cancer cell line. The cell may be a bacterial, yeast, plant or animal cell. Preferably, the cell is an animal cell. A preferred animal cell is a vertebrate cell, preferably a rodent or primate cell, preferably a mouse or human cell. The cell can be, or can be part of, a cell culture, a cell line, a biopsy and an organoid. Preferably, the cell is part of or derived from a patient derived tissue, preferably a cultured patient-derived tissue. The cell may be part of or derived from a biopsy or an organoid, preferably a tumor organoid. The biopsy can be an excisional biopsy, an incisional biopsy or core biopsy. The organoid is preferably a cancer organoid. The organoid is preferably a patient-derived organoid, preferably a tumor organoid.

A preferred cell is a human cell, preferably at least one of a cancer cell, an immune cell and a neural cell. A preferred cell is a human cell line, preferably a human cancer cell line, a human immune cell line and/or a human neural cell line. The cell line may be an immortalized cell line. Preferably the cell is a HEK293T cell.

The provided cell may express the membrane-bound protein and/or the transmembrane E3 ubiquitin ligase at endogenous levels or may be modified to induce or increase expression of the membrane-bound protein and/or the transmembrane E3 ubiquitin ligase. In addition or alternatively, the cell may be modified to express a transmembrane E3 ubiquitin ligase comprising one or more non-native epitope tags as defined herein and/or a membrane-bound protein comprising one or more non-native epitope tags as defined herein. The (non-native) epitope-comprising transmembrane E3 ubiquitin ligase and the (non-native) epitope comprising membrane-bound protein are preferably expressed in the same cell.

The provided cell, preferably a cell line, may express at least one of a wild type or “native” transmembrane E3 ubiquitin ligase and a wild type membrane-bound protein. At least one of the wild type transmembrane E3 ubiquitin ligase and the wild type membrane-bound protein may be overexpressed in the cell. The cell may transiently overexpress at least one of a wild type transmembrane E3 ubiquitin ligase and a wild type membrane-bound protein. Optionally, at least one of the wild type transmembrane E3 ubiquitin ligase and the wild type membrane-bound protein may be permanently overexpressed in the cell.

Alternatively or in addition, the provided cell, preferably a cell line, may express at least one of an engineered transmembrane E3 ubiquitin ligase and an engineered membrane-bound protein. The engineered transmembrane E3 ubiquitin ligase comprises a first, and optional fourth, non-native epitope tag as defined herein. The engineered membrane-bound protein comprises a second, and optional third, non-native epitope tag as defined herein. The provided cell may transiently overexpress at least one of an engineered transmembrane E3 ubiquitin ligase and an engineered membrane-bound protein. Optionally, the cell may permanently overexpress at least one of an engineered transmembrane E3 ubiquitin ligase and an engineered membrane-bound protein.

Expression, optionally permanent expression, of at least one of the engineered transmembrane E3 ubiquitin ligase and the engineered membrane-bound protein can be achieved using any conventional means known in the art by the skilled person. For example, permanent expression may be accomplished by e.g. integrating an expression cassette expressing at least one of the (engineered) transmembrane E3 ubiquitin ligase and the (engineered) membrane-bound protein into the genome of the cell.

Expression of the (optionally non-native epitope(s) comprising) transmembrane E3 ubiquitin ligase may be controlled by its native promoter or by a non-native promoter, such as, but not limited to, a constitutively active promoter. The expression of the (optionally non-native epitope(s) comprising) membrane-bound protein may be controlled by its native promoter or by a non-native promoter, such as, but not limited to, a constitutively active promoter.

The sequence encoding at least one of the (optionally non-native epitope(s) comprising) transmembrane E3 ubiquitin ligase and the (optionally non-native epitope(s) comprising) membrane-bound protein can be introduced into the provided cell for transient or permanent expression. Optionally, the coding sequence(s) are comprised in an expression cassette that is introduced into the cell. The expression cassette preferably further comprises one or more elements controlling the expression of the transmembrane E3 ubiquitin ligase and/or controlling the expression the membrane-bound protein. A preferred expression element is a native promoter or a non-native promoter. The expression cassette may be part of an expression vector. A preferred expression vector is a naked DNA, a DNA complex or a viral vector. A preferred naked DNA is a linear or circular nucleic acid molecule, e.g. a plasmid. A plasmid refers to a circular double stranded DNA loop into which additional DNA segments can be inserted, such as by standard molecular cloning techniques. A DNA complex can be a DNA molecule coupled to any carrier suitable for delivery of the DNA into the cell. A preferred carrier is selected from the group consisting of a lipoplex, a liposome, a polymersome, a polyplex, a viral vector, a dendrimer, an inorganic nanoparticle, a virosome and cell-penetrating peptides.

A provided cell may have been modified to express a non-native epitope tag(s)—comprising transmembrane E3 ubiquitin ligase and a non-native epitope tag(s)—comprising membrane-bound protein at endogenous levels. As non-limiting example, a sequence encoding a first, and optional fourth, non-native epitope tag may be incorporated into the genomic sequence of the provided cell. In addition in the same cell, a sequence encoding a second, and optional third, non-native epitope tag may be incorporated into the genomic sequence of the provided cell.

Thus a genomic sequence of the provided cell encoding the transmembrane E3 ubiquitin ligase can be modified to incorporate a sequence encoding the first, and optional fourth, non-native epitope tag. The modified genomic sequence preferably encodes and expresses a transmembrane E3 ubiquitin ligase comprising a first, and optional fourth, non-native epitope tag as defined herein. Preferably in the same provided cell, a genomic sequence encoding the membrane-bound protein can be modified to incorporate a sequence encoding a second, and optional third, non-native epitope tag. The modified genomic sequence preferably encodes and expresses a membrane-bound protein comprising a second, and optional third, non-native epitope tag as defined herein.

Methods for targeted genomic modification to incorporate a sequence encoding a first, second and optional third and fourth, non-native epitope tag are well known to the person skilled in the art and include, but are not limited to, a site-directed endonuclease that generates a double-stranded break at the genomic location to incorporate a sequence encoding the first, and optional fourth, non-native epitope tag, or to incorporate a sequence encoding a second, and optional third, non-native epitope tag. A preferred site-directed nuclease is a CRISPR-Cas system. Preferably, the generated double-stranded break is unique for a single location in the genome. Alternatively double-stranded breaks may be generated at two or more genomic locations, wherein at least one of the double-stranded breaks is in or nearby the sequence encoding a transmembrane E3 ubiquitin ligase or in or nearby the sequence encoding the membrane-bound protein, to incorporate the sequence encoding respectively the first or second, and optional third and/or fourth tag.

The skilled person straightforwardly understands that additional sequences may be incorporated into the genome, such as, but not limited to, a selection cassette to select for the modified cells. Such selection cassette is preferably incorporated in an intergenic or intronic region, preferably an intron of the transmembrane ubiquitin E3 ligase and/or an intron of the membrane-bound protein.

A first, and optional fourth, non-native epitope tag may thus be introduced into the genome of a cell by the step of introducing into a cell i) a site directed nuclease generating a double-stranded break in or nearby the sequence encoding a transmembrane E3 ubiquitin ligase, and ii) an oligonucleotide or donor plasmid comprising a sequence encoding a first and optional fourth tag. The double-stranded break is preferably located at a location such that the mature transmembrane E3 ubiquitin ligase comprises the first and optional fourth tag. Preferably the double-stranded break is located at a location such that the first, and optional fourth tag, is positioned in between a signal peptide and the mature transmembrane ubiquitin E3 ligase. Preferably the double-stranded break is located at a location such that the first and optional fourth tag, is positioned extracellularly. Preferably the double-stranded break is located at a location such that at least one of the first and optional fourth tag, is positioned at or nearby the N-terminus of the mature transmembrane ubiquitin E3 ligase. Alternatively or in addition, at least one of the first and optional fourth tag may be located at or nearby the C-terminus of the mature transmembrane ubiquitin E3 ligase. Alternatively or in addition, at least one of the first and optional fourth tag may be located in an extracellular loop region of the mature transmembrane ubiquitin E3 ligase.

The cell expressing the transmembrane ubiquitin ligase comprising the first, and optional fourth, non-native epitope tag may be used in the screening method as defined herein. In this embodiment, the heterobifunctional molecule may comprise a first binding domain for specific binding to the first non-native eptitope tag and a second binding domain is capable of binding a native epitope present in the wild type transmembrane protein.

Preferably in the same cell, a second, and optional third, non-native epitope tag may be introduced into the genome of a cell by the step of introducing into a cell i) a site-directed nuclease generating a double-stranded break in or nearby the sequence encoding a membrane-bound protein, and ii) an oligonucleotide or donor plasmid comprising a sequence encoding a second and optional third tag. Preferably the double-stranded break is located at a location such that the second and optional third tag, is positioned extracellularly. The double-stranded break is preferably located at a location such that the mature membrane-bound protein comprises the second and optional third tag at the N-terminus. Alternatively or in addition, the at least one of the second and optional third tag may be located at or nearby the C-terminus of the membrane-bound protein. Alternatively or in addition, the at least one of the second and optional third tag may be located in an extracellular loop region of the membrane-bound protein.

The oligonucleotide or donor plasmid preferably comprises sequences to facilitate homology-directed repair.

Alternatively or in addition, the first, second, and optional third and fourth, non-native epitope tags may be introduced into the genome using the CRISPR-Cas prime editing technology.

Optionally the CRISPR technology, such as the above-described CRISPR-technologies, may be used to generate an appropriate control for the method as defined herein, such as, but not limited to, the generation of a transmembrane E3 ubiquitin ligase lacking a functional ligase domain.

### Step b): Exposing the Cell to a Heterobifunctional Molecule

Step b) of exposing the cell to a heterobifunctional molecule is preferably under the conditions allowing a heterobifunctional molecule to simultaneously bind the transmembrane E3 ubiquitin ligase and the transmembrane protein. Such conditions are well-known to the person skilled in the art. As a non-limiting example, the heterobifunctional molecule may be straightforwardly added to the cell culture medium.

The concentration of the heterobifunctional molecule used in step b) of the method of the invention may vary, e.g. the concentration may be dependent on the heterobifunctional molecule and/or the epitopes present in the transmembrane E3 ubiquitin ligase and/or the membrane-bound protein. The concentration of the heterobifunctional molecule may be determined experimentally using standard techniques. Preferably the concentration of the heterobifunctional molecule exposed to the cell is about 0.1 nM-1000 nM, about 0.5 nM-500 nM, about 5 nM-100 nM, about 20 nM-80 nM, or about 40 nM-60 nM. The concentration of the heterobifunctional molecule is preferably about 50 nM. The heterobifunctional molecule as described herein is capable of simultaneous binding to a transmembrane E3 ubiquitin ligase and a membrane-bound protein. The transmembrane E3 ubiquitin ligase is preferably a transmembrane E3 ubiquitin ligase as described herein.

The membrane-bound protein is preferably a transmembrane protein. The transmembrane protein may be at least one of a type I, type II and type III transmembrane protein. The transmembrane protein may be a so-called “multispan” protein. The transmembrane protein may be a transmembrane protein as described herein.

The time period for exposing exposing the cell to a heterobifunctional molecule is preferably at least about 0.5, 1, 2, 3, 4, 5, 6, 7, 8, 9, 10, 11, 12, 14, 16, 18, 20, 22 or 24 hours, or preferably at least 1, 2, 3, 4, 5, 6 or 7 days.

At least one of the transmembrane E3 ubiquitin ligase and the membrane-bound protein may be a wild type protein, e.g. a protein that is naturally encoded in the genome and optionally present (expressed) in the provided cell. The transmembrane E3 ubiquitin ligase and the membrane-bound protein are preferably expressed in the same cell. Optionally, at least one of the wild type proteins is overexpressed in the provided cell. Thus optionally, the wild type transmembrane E3 ubiquitin ligase for use in the method of the invention has an induced or increased expression in the provided cell. Optionally, the wild type membrane-bound protein for use in the method of the invention has an induced or increased expression in the provided cell. The heterobifunctional molecule for use in the method of the invention preferably comprises a first binding domain capable of binding a (native) epitope present in the wild type transmembrane E3 ubiquitin ligase and/or comprises a second binding domain capable of binding a (native) epitope naturally present in the wild type membrane-bound protein, preferably a wild type transmembrane protein.

Alternatively, at least one of the transmembrane E3 ubiquitin ligase and the membrane-bound protein is not a wild type protein. Preferably, the transmembrane E3 ubiquitin ligase comprises a first non-native epitope tag. Preferably, the first non-native epitope tag is located in the extracellular portion of the ubiquitin ligase. Thus preferably, the first non-native epitope tag is exposed on the cell surface of the provided cell. Preferably, the first non-native epitope tag is located at or nearby
i) the N-terminus of the transmembrane E3 ubiquitin ligase;
ii) the C-terminus of the transmembrane E3 ubiquitin ligase; and/or
ii) an extracellular loop region of the transmembrane E3 ubiquitin ligase.

When the transmembrane E3 ubiquitin ligase comprises a first non-native epitope tag, the heterobifunctional molecule preferably comprises a first binding domain that selectively binds to the first non-native epitope tag.

Preferably, the membrane-bound protein comprises a second non-native epitope tag.

Preferably, the second non-native epitope tag is located in the extracellular portion of the membrane-bound protein. Thus preferably, the second non-native epitope tag is exposed on the cell surface of the provided cell.

Preferably, the second non-native epitope tag is located at or nearby
i) the N-terminus of the membrane-bound protein;
ii) the C-terminus of the membrane-bound protein; and/or
iii) an extracellular loop region of the membrane-bound protein.

When the membrane-bound protein comprises a second non-native epitope tag, the heterobifunctional molecule preferably comprises a second binding domain that selectively binds to the second non-native epitope tag.

In a preferred method of the invention the cell is exposed to a heterobifunctional molecule, wherein the heterobifunctional molecule comprises:

i) a first binding domain capable of specific binding to a first non-native epitope tag located in an extracellular portion of the transmembrane E3 ubiquitin ligase; and

ii) a second binding domain capable of specific binding to a second non-native epitope tag located in an extracellular portion of the membrane-bound protein.

A “non-native epitope tag” is understood herein as an epitope that is not normally present in the wild type, naturally occurring, protein. The terms “epitope” and “epitope tag” may be used interchangeably herein.

The first non-native epitope tag is preferably located in the extracellular portion of the transmembrane E3 ubiquitin ligase. In case the transmembrane E3 ubiquitin ligase comprises an extracellular N-terminal portion the first epitope, preferably the first non-native epitope, is preferably located at, or nearby, the original N-terminus of the transmembrane E3 ubiquitin ligase. Optionally there are additional amino acid residues located in between the original N-terminus and the first epitope. Optionally, there are about 10-100, 5-50 or about 1-10 amino acid residues located in between the original N-terminus of the transmembrane E3 ubiquitin ligase and the first, optionally non-native, epitope. These amino acid residues may be native to the transmembrane E3 ubiquitin ligase or the transmembrane E3 ubiquitin ligase may be further extended by these additional amino acid residues.

In case the transmembrane E3 ubiquitin ligase comprises an extracellular C-terminal portion, the first epitope, preferably the first non-native epitope, is preferably located at, or nearby, the original C-terminus of the transmembrane E3 ubiquitin ligase. Optionally there are additional amino acid residues located in between the original C-terminus and the first epitope. Optionally, there are about 10-100, 5-50 or about 1-10 amino acid residues located in between the original C-terminus of the transmembrane E3 ubiquitin ligase and the first, optionally non-native, epitope. These amino acid residues may be native to the transmembrane E3 ubiquitin ligase or the transmembrane E3 ubiquitin ligase may be further extended by these additional amino acid residues.

Independent of whether (or not) the transmembrane E3 ubiquitin ligase comprises a N-terminal and/or C-terminal extracellular portion, the first, optionally non-native, epitope tag may alternatively or in addition be located in an extracellular loop region of the transmembrane E3 ubiquitin ligase.

Alternatively or in addition, the first non-native epitope tag may be extended by one or more amino acid residues that are located adjacent to an intracellular portion of the transmembrane E3 ubiquitin ligase, such as, but not limited to, located at or nearby the original intracellular N-terminus and/or C-terminus. In this embodiment, the non-native epitope tag may be extended by a membrane spanning domain, wherein the membrane spanning domain causes extracellular expression of the tag, preferably a peptide-tag or a protein-tag as defined herein. Non-limiting examples of such extended tags are described in WO2012116076 and Brown et al (PLoS One, 2013 Sep. 2; 8(9):e73255 (the “Snorkel tag”)), which are incorporated herein by reference. The membrane spanning domain extending the non-native epitope tag preferably has a sequence as depicted in FIG. 1 of Brown et al (supra). Snorkel tag preferably has a sequence as depicted in FIG. 1 of Brown et al (supra).

The second non-native epitope tag is preferably located in the extracellular portion of the membrane-bound protein. In case the membrane-bound protein comprises an extracellular N-terminal portion the second epitope, preferably the second non-native epitope, is preferably located at, or nearby, the original N-terminus of the membrane-bound protein. Optionally there are additional amino acid residues located in between the original N-terminus and the second epitope. Optionally, there are about 10-100, 5-50 or about 1-10 amino acid residues located in between the original N-terminus of the membrane-bound protein and the second, optionally non-native, epitope. These amino acid residues may be native to the membrane-bound protein, or the membrane-bound protein may be further extended by these additional amino acid residues.

In case the membrane-bound protein comprises an extracellular C-terminal portion, the second epitope, preferably the second non-native epitope, is preferably located at, or nearby, the original C-terminus of the membrane-bound protein. Optionally there are additional amino acid residues located in between the original C-terminus and the second epitope. Optionally, there are about 10-100, 5-50 or about 1-10 amino acid residues located in between the original C-terminus of the membrane-bound protein and the second, optionally non-native, epitope. These amino acid residues may be native to the membrane-bound protein or the membrane-bound protein may be further extended by these additional amino acid residues.

Independent of whether (or not) the membrane-bound protein comprises a N-terminal and/or C-terminal extracellular portion, the second, optionally non-native, epitope tag may alternatively or in addition be located in an extracellular loop region of the membrane-bound protein.

Alternatively or in addition, the second non-native epitope tag may be extended by one or more amino acid residues that are located adjacent to an intracellular portion of the membrane-bound protein, such as, but not limited to, located at or nearby the original intracellular N-terminus and/or C-terminus. In this embodiment, the non-native epitope may be extended by a membrane spanning domain, wherein the membrane spanning domain causes extracellular expression of the tag, preferably a peptide-tag or protein-tag as defined herein. Non-limiting examples of such extended tags are described in WO2012116076 and Brown et al (PLoS One, 2013 Sep. 2; 8(9):e73255 (the “Snorkel tag”)), which are incorporated herein by reference. The membrane spanning domain extending the tag preferably has a sequence as depicted in FIG. 1 of Brown et al (supra). Snorkel tag preferably has a sequence as depicted in FIG. 1 of Brown et al (supra).

Incorporating the non-native epitope tag into respectively the transmembrane E3 ubiquitin ligase and the membrane-bound protein can be done using any conventional molecular biology technique known in the art. The first and second epitope tag can be any suitable tag. The epitope tag may be a linear or conformational epitope. The tag is preferably a peptide tag or protein tag. Preferably, the tag is a short amino acid sequence. The length of the first and/or second non-native epitope tag is preferably between about 2-50, 3-40, 4-30, 5-20 or 8-15 amino acid residues, Preferably, the tag is an amino acid sequence against which an antibody, or antibody fragment, preferably a nanobody, can be raised using any conventional means known to the skilled person. The non-native epitope tag can be a publicly available tag or a newly discovered sequence. The first and second epitope tags may be the same or different tags. Preferably, the first and second epitope tags are different tags.

The first and/or second non-native epitope tag may be a peptide tag selected from the group consisting of Alpha tag, E6 tag, V5 tag, VSV-tag, AviTag, C-tag, Calmodulin-tag, polyglutamate tag, polyarginine tag, E-tag, FLAG-tag, HA-tag, His-tag, Myc-tag, NE-tag, Rho1D4-tag, S-tag, SBP-tag, Softag 1, Spot-tag, Strep-tag, T7-tag, TC tag, Ty tag and Xpress tag

The first and/or second non-native epitope tag may be a protein tag selected from the group consisting of GFP-tag (Green fluorescent protein), RFP-tag (red fluorescent protein), YFP-tag (yellow fluorescent protein), BFP-tag (blue fluorescent protein), BCCP-tag (Biotin Carboxyl Carrier Protein), Glutathione-S-transferase-tag, HaloTag, SNAP-tag, CLIP-tag, HUH-tag Maltose binding protein-tag, Nus-tag, Thioredoxin-tag, Fc-tag, Carbohydrate Recognition Domain (CRD) and CRDSAT-tag.

The first and/or second non-native epitope tag may be extended by one or more amino acid residues that cause extracellular expression of the tag. The part that causes extracellular expression is preferably a membrane-spanning domain, preferably a transmembrane domain (TMD), preferably a TMD as depicted in FIG. 1 of Brown et al (supra). The membrane-spanning domain may result in the extracellular expression of a protein- or peptide-tag as defined herein.

The membrane-spanning domain may result in the extracellular expression of a 2, 3, 4, 5, 6, 7, 8, 9, 10 or more protein- or peptide-tag as defined herein. Preferably, the membrane-spanning domain may result in the extracellular expression of at least one of a myc-tag, a FLAG-tag, an alpha-tag and a E6 tag. Preferably, the membrane-spanning domain may result in the extracellular expression of at least an E6 tag and a FLAG-tag. Alternatively or in addition, the membrane-spanning domain may result in the extracellular expression of at least an alpha tag and a myc-tag.

The non-native epitope tag may be selected from the group consisting of an Alpha tag, an E6 tag, a myc tag, a FLAG tag, a His tag, a V5-tag, a VSV-tag, a GFP-tag and a RFP-tag.

The first epitope tag may be an Alpha tag, preferably as described in Gotzke et al (2019, Nature Communications, 10(1), 1-12). Alternatively, the first epitope tag may be an UBC6e tag (E6 tag), as described in Ling et al. (2019, Molecular Immunology, 114(July), 513-523). The second epitope tag may be an Alpha tag, preferably as described in Götzke et al (supra). Alternatively, the second epitope tag may be an UBC6e tag (E6 tag), as described in Ling et al. (supra). Preferably, the first tag may be an Alpha tag and the second tag may be a E6 tag. Alternatively, the first tag may be an E6 tag and the second tag may be an Alpha tag.

Any suitable combination of an epitope tag and the corresponding antibody, or antibody fragment, recognizing said epitope may be used in the method as defined herein. A preferred antibody fragment is a nanobody. Thus any suitable combination of an epitope and the corresponding nanobody recognizing said epitope may be used in the method as defined herein.

The skilled person is capable of selecting a suitable epitope—antibody, or antibody fragment, combination. For example, the skilled artisan may select a suitable epitope—antibody, or antibody fragment, combination that is known in the art. Alternatively or in addition, the epitope-antibody, or antibody fragment, combination may be a newly discovered combination and may be used in the method as defined herein.

The antibody, or antibody fragment, may be any suitable antibody, or antibody fragment, specifically binding to the first or the second epitope tag. A preferred antibody fragment is a nanobody. Preferably, the heterobifunctional molecule for use in the method of the invention is a bi-specific antibody, preferably a bi-specific nanobody.

Preferably, in case the first epitope tag is an Alpha tag and the second epitope tag is an E6 tag, the first binding domain of the heterobifunctional molecule may comprise an anti-Alpha VHH and the second binding domain may comprise an anti-E6 VHH. Preferably, in case the first epitope tag is an E6 tag and the second epitope tag is an Alpha tag, the first binding domain of the heterobifunctional molecule may comprise an anti-E6 VHH (Ling et al, supra) and the second binding domain may comprise an anti-Alpha VHH (Gotzke et al, supra). Hence a preferred epitope tag—binding domain combination is at least one of:

i) Alpha tag—anti-Alpha VHH (see e.g. Gotzke et al (supra); and

ii) E6 tag—anti-E6 VHH (see e.g. Ling et al, supra)

A preferred alpha tag has at least about 80%, 85%, 90%, 95%, 96%, 97%, 98%, 99% or 100% sequence identity with SEQ ID NO:96. A preferred E6 tag has at least about 80%, 85%, 90%, 95%, 96%, 97%, 98%, 99% or 100% sequence identity with SEQ ID NO:97. A preferred anti-alpha VHH has at least about 80%, 85%, 90%, 95%, 96%, 97%, 98%, 99% or 100% sequence identity with SEQ ID NO:98. A preferred CDR3 sequence of the anti-E6 VHH has at least about 80%, 85%, 90%, 95%, 96%, 97%, 98%, 99% or 100% sequence identity with SEQ ID NO:99.

The membrane-bound protein may comprise a third non-native epitope tag. The third non-native epitope tag may be used to determine the protein level of the membrane-bound protein, preferably to determine at least one of the protein cell surface level, the total protein level and the intracellular levels of the membrane-bound protein. The third non-native epitope tag is preferably located in the extracellular portion of the membrane-bound protein. Thus preferably, the third non-native epitope tag is exposed on the cell surface of the provided cell. Preferably, the third non-native epitope tag is located at or nearby:

i) the N-terminus of the membrane-bound protein;

ii) the C-terminus of the membrane-bound protein; and/or

iii) an extracellular loop region of the membrane-bound protein.

The location of the third non-native epitope tag may be as indicated above for the second epitope tag. The third non-native epitope tag may be located N-terminal and/or C-terminal of the second (non-native) epitope tag.

The transmembrane E3 ubiquitin ligase may comprise a fourth non-native epitope tag. The fourth non-native epitope tag may be used to determine the protein level of the transmembrane E3 ubiquitin ligase, preferably to determine at least one of the protein cell surface level, the total protein level and the intracellular levels of the transmembrane E3 ubiquitin ligase. The fourth non-native epitope tag is preferably located in the extracellular portion of the transmembrane E3 ubiquitin ligase. Thus preferably, the fourth non-native epitope tag is exposed on the cell surface of the provided cell. Preferably, the fourth non-native epitope tag is located at or nearby

i) the N-terminus of the transmembrane E3 ubiquitin ligase;

ii) the C-terminus of the transmembrane E3 ubiquitin ligase; and/or

ii) an extracellular loop region of the transmembrane E3 ubiquitin ligase.

The location of the fourth non-native epitope tag may be as indicated above for the first epitope tag. The fourth non-native epitope tag may be located N-terminal and/or C-terminal of the first (non-native) epitope tag.

Hence in an embodiment, the transmembrane E3 ubiquitin ligase may comprise a first and a fourth non-native epitope tag and the membrane-bound protein may comprise a second and a third non-native epitope tag. The third and/or fourth non-native epitope tag may be any conventional tag known to the skilled person for protein detection, such as, a peptide-tag or a protein-tag.

Optionally, the peptide tag selected from the group consisting of Alpha tag, E6 tag, V5 tag, VSV-tag, AviTag, C-tag, Calmodulin-tag, polyglutamate tag, polyarginine tag, E-tag, FLAG-tag, HA-tag, His-tag, Myc-tag, NE-tag, Rho1D4-tag, S-tag, SBP-tag, Softag 1, Spot-tag, Strep-tag, T7-tag, TC tag, Ty tag and Xpress tag. Optionally the protein tag selected from the group consisting of GFP-tag (Green fluorescent protein), RFP-tag (red fluorescent protein), YFP-tag (yellow fluorescent protein), BFP-tag (blue fluorescent protein), BCCP-tag (Biotin Carboxyl Carrier Protein), Glutathione-S-transferase-tag, HaloTag, SNAP-tag, CLIP-tag, HUH-tag Maltose binding protein-tag, Nus-tag, Thioredoxin-tag, Fc-tag, Carbohydrate Recognition Domain (CRD) and CRDSAT-tag.

The third non-native epitope tag may be selected from the group consisting of a myc-tag, his-tag, FLAG-tag, V5-tag, VSV-tag, HA-tag, GFP and RFP. The fourth non-native epitope tag may be selected from the group consisting of a myc-tag, his-tag, FLAG-tag, V5-tag, VSV-tag, HA-tag, GFP and RFP.

A preferred combination of the first and fourth tag are a myc-tag and an Alpha tag, a myc-tag and an E6 tag, a FLAG tag and an Alpha tag, or a FLAG tag and an E6 tag. A preferred combination of a second and third tag are a Flag tag and an E6 tag, a FLAG tag and an Alpha tag, a myc-tag and a E6 tag or a myc tag and an Alpha tag.

The third and/or fourth non-native epitope tag may further comprise a part that causes extracellular expression of the tag. The part that causes extracellular expression is preferably a membrane-spanning domain, preferably a transmembrane domain (TMD), preferably a TMD as depicted in FIG. 1 of Brown et al (supra). The extracellularly expressed tag is preferably a peptide-tag or a protein-tag as defined herein.

### Step c).: Determining the Surface Level of the Membrane-Bound Protein

The level, or amount, of the membrane-bound protein may be determined using any conventional means known to the skilled person. Such means include, but are not limited to microscopy, western blotting, optionally quantitative immunofluorescence and/or quantitative western blotting, cell surface biotinylation, FACS analysis, labelling of the membrane-bound protein using cell-impermeable fluorescent probes (e.g. SNAP labelling), and quantitative mass spectrometry.

The absolute amount or level of the membrane-bound protein may be determined, e.g. by direct comparison between the membrane-bound protein level before or after exposure to the heterobifunctional molecule, such as by determining the fluorescence intensity at the cell surface by e.g. either fluorescence microscopy or FACS analysis before and after exposure.

For microscopy-based analysis, one can label the surface-localized membrane-bound proteins using a fluorescence label for the membrane-bound protein, preferably in non-permeabilized cells. The total number of cells can be determined, e.g. by staining the nuclei of cells with DAPI or similar dyes. The mean fluorescence intensity values from multiple images taken with identical magnification, laser power, optical settings and exposure times can be analyzed in ImageJ or similar analysis programs. Each image is preferably normalized to the number of stained nuclei, e.g. normalized to its own DAPI value, and the resulting value represents the relative amount of membrane-bound proteins at the surface per cell.

Alternatively or in addition, one can use dual labeling of the membrane-bound protein, using first a fluorescence labeling for the surface-localized membrane-bound protein in non-permeabilized cells using a first epitope tag and subsequently a fluorescent labeling of the total membrane-bound protein using a second epitope tag in permeabilized cells. For analysis by microscopy, the fluorescence intensity values from multiple images taken with identical magnification, laser power, optical settings and exposure times can be analyzed in ImageJ or similar analysis programs. Background levels from non-transfected cells is preferably subtracted for each image and the intensity values is preferably averaged for each condition. The ratio of the mean fluorescence intensities of the surface-localized proteins versus the mean fluorescence intensity of the total (membrane-bound) protein is a measure of the relative amount of membrane-bound proteins at the surface (as described e.g. in Stüber et al, ACS Chem Biol, 2019, 14(6):1154-1163)

Alternatively or in addition, for analysis using flow cytometry dead cells and non-cellular components are preferably excluded by the Forward and Side Scatter plots and based on a negative DNA staining. Subsequently, doublet cells are preferably excluded using the Forward Side scatter area vs height. The fluorescence signals can be quantified based on the threshold set e.g. based on the fluorescence intensities determined for non-transfected cells using FACSdiva, FlowJo or similar analysis programs. The ratio of the mean fluorescence intensities of the surface-localized proteins versus the mean fluorescence intensity of the total (membrane-bound) protein is a measure of the relative amount of membrane-bound proteins at the surface (Stub& et al, supra).

Alternatively or in addition, the surface levels of the membrane-bound protein may be determined based on the mature versus immature forms of the protein. Membrane-bound proteins may undergo complex glycosylation during biosynthesis and additional posttranslational modifications resulting in distinct molecular weights (MW) as visualized e.g. by Western Blotting with the higher MW representing mature forms that are present at the cell surface. The mean pixel intensities of the bands representing the mature and the immature forms of the membrane-bound proteins can be determined by Western blot analysis software e.g. including ImageQuant (Zeiss), ImageJ or similar programs. The ratio of the intensities of mature forms versus the total amount of membrane-bound proteins is a measure of the relative amount of membrane-bound proteins at the surface. (as described e.g. in Koo et al 2021, Nature, 2012; 488(7413):665-9).

Alternatively or in addition, the relative level, or amount, of the membrane-bound protein may be determined, e.g. by comparison to a household protein or total cell protein levels before and after exposure, e.g. using the above mentioned Western blot analysis.

Alternatively or in addition, the surface levels of the membrane-bound protein may be determined by surface biotinylation. To this extend surface proteins are labeled with cell-impermeable Biotin after which all surface proteins are isolated by a Streptavidin pull down. Subsequently, the pools of total and surface-localized proteins are analyzed, e.g. using Western blotting. The ratio of the intensities of the membrane-bound proteins in the surface pool versus the intensity of the membrane-bound protein in the total protein pool, determined by analysis software, such as ImageQuant (Zeiss), ImageJ or similar programs, is a measure of the relative amount of membrane-bound proteins at the surface. (Dubey et al, Elife, 2020; 9:e54469; Hao et al, Nature 2012; 485(7397): 195-200).

Alternatively or in addition, the decrease in protein level of the membrane-bound protein may be determined by comparing different combinations of the membrane-bound protein and a transmembrane ubiquitin E3 ligase. As a non-limiting example, the protein level determined after forced dimerization of a membrane-bound protein and a first transmembrane ubiquitin E3 ligase may be compared with the protein level determined after forced dimerization of the same membrane-bound protein and a second transmembrane ubiquitin E3 ligase. Such method would allow for the determination of the most effective combination of the membrane-bound protein and the transmembrane ubiquitin E3 ligase, e.g. determination of the transmembrane ubiquitin E3 ligase that causes the strongest decrease of the surface level of the membrane-bound protein upon forced dimerization.

The transmembrane ubiquitin E3 ligase that causes the strongest decrease in the surface level of the membrane-bound protein upon forced dimerization preferably decreases the surface level at least about 10%, 20%, 30%, 40%, 50%, 60%, 70%, 80%, 90%, 95% or 100%, as compared to the transmembrane ubiquitin E3 ligase that causes the weakest, or not any, decrease in the surface level of the membrane-bound protein upon forced dimerization.

The method of the invention can be straightforwardly performed in a multiplexed manner. As a non-limiting example, a first cell can be contacted by a first heterobifunctional molecule and a second cell can be contacted by a second heterobifunctional molecule. The first and second cell are preferably physically separated, e.g. by maintaining them in separate wells. The first and second cell may have the same genetic background. The first and the second cell can be identical cells.

The first cell may be exposed to a first heterobifunctional molecule and a second cell may be exposed to a second heterobifunctional molecule. The first and second heterobifunctional molecule may differ in their first binding domain and/or in their second binding domain.

The first and second heterobifunctional molecule may differ in their first binding domain and their second binding domain.

The first and second heterobifunctional molecule may differ in their first binding domain, but do not differ in their second binding domain. The first binding domain of the first heterobifunctional molecule may selective bind to a native or non-native epitope tag located in a first transmembrane E3 ubiquitin ligase. The first binding domain of the second heterobifunctional molecule may selective bind to a native or non-native epitope tag located in a second transmembrane E3 ubiquitin ligase. The second binding domain of the first and second heterobifunctional molecule may selectively bind to a native or non-native epitope tag located in the membrane-bound protein. This allows for a straightforward approach to determine the transmembrane ubiquitin E3 ligase that causes the strongest, i.e. most effective, decrease in the surface level of the membrane-bound protein upon forced dimerization.

The first and second heterobifunctional molecule may differ in their second binding domain, but do not differ in their first binding domain. The second binding domain of the first heterobifunctional molecule may selective bind to a native or non-native epitope tag located in a membrane-bound protein. The second binding domain of the second heterobifunctional molecule may selective bind to a native or non-native epitope tag located in a second membrane-bound protein. The first binding domain of the first and second heterobifunctional molecule preferably binds to a native or non-native epitope tag located in the transmembrane E3 ubiquitin ligase. This allows for a straightforward approach to determine the membrane-bound protein that is effectively decreased upon forced dimerization with a certain transmembrane E3 ubiquitin ligase.

Alternatively, the first and second heterobifunctional molecule do not differ in their first and second binding domain, but the first cell expresses a first transmembrane ubiquitin E3 ligase comprising a non-native epitope tag that can be bound by the first binding domain of the heterobifunctional molecule, and the second cell expresses a second transmembrane ubiquitin E3 ligase comprising the same non-native epitope tag. The first and second cell preferably express a membrane-bound protein comprising a native or non-native epitope tag that can be bound by the second binding domain of the heterobifunctional molecule. The first and second transmembrane ubiquitin E3 ligases are preferably two different transmembrane ubiquitin E3 ligases. The first transmembrane ubiquitin E3 ligase is preferably selected from the group consisting of RNF43, RNF167, ZNRF3, RNF13, AMFR, MARCH1, MARCH2, MARCH4, MARCH8, MARCH9, RNF149, RNF145, RNFT1, RNF130 and RNF128. The second transmembrane ubiquitin E3 ligase is preferably selected from the group consisting RNF43, RNF167, ZNRF3, RNF13, AMFR, MARCH1, MARCH2, MARCH4, MARCH8, MARCH9, RNF149, RNF145, RNFT1, RNF130 and RNF128.

Alternatively, the first and second heterobifunctional molecule do not differ in their first binding domain and their second binding domain, but the first cell expresses a first membrane-bound protein comprising a non-native epitope tag that can be bound by the second binding domain of the heterobifunctional molecule, and the second cell expresses a second membrane-bound protein comprising same non-native epitope tag. The first and second cell preferably express a transmembrane E3 ubiquitin ligase comprising a native or non-native epitope tag that can be bound by the first binding domain of the heterobifunctional molecule. The first and second membrane-bound protein are preferably two different membrane-bound proteins.

The skilled person straightforwardly understands that the first and second cell can be straightforwardly extended to a third cell, a fourth cell, a fifth cell, etcetera. Equally, the first and second heterobifunctional molecule can be straightforwardly extended to a third heterobifunctional molecule, a fourth heterobifunctional molecule, a fifth heterobifunctional molecule etcetera.

Preferably, the method of the invention comprises a step a) wherein a first and a second cell is provided, wherein
the first cell expresses a first transmembrane E3 ubiquitin ligase and a first membrane-bound protein at its cell surface; and
the second cell expresses a second transmembrane E3 ubiquitin ligase and the first membrane-bound protein and its cell surface,

wherein the first and second transmembrane E3 ubiquitin ligase are different ligases comprising the same first extracellular non-native epitope tag;

wherein in step b) the first and the second cell is exposed the heterobifunctional molecule,

wherein the heterobifunctional molecule comprises:

i) a first binding domain capable of specific binding to the first non-native epitope tag; and
ii) a second binding domain capable of specific binding to an extracellular portion of the membrane-bound protein, preferably to the second non-native epitope tag; and

wherein in step c) the surface level of the membrane-bound protein of the first and second cell are determined. A combination is preferably effective when the cell surface levels of the membrane-bound protein in the first cell are decreased at least about 2%, 5%, 10%, 20%, 30%, 40%, 50%, 60, 70%, 80%, 90%, 95% or about 100% as compared to the cell surface levels of the membrane-bound protein in the second cell after step b). The effective combination is the combination of the first transmembrane E3 ubiquitin ligase and the membrane-bound protein.

Upon simultaneous binding to the heterobifunctional molecule, one combination of a transmembrane E3 ubiquitin ligase and a membrane-bound protein preferably results in a stronger decrease in the surface level of the membrane-bound protein than a combination of another transmembrane E3 ubiquitin ligase and the (same) membrane-bound protein. This more effective combination is herein indicated as the combination in “the first cell”. The skilled person however readily understands that the method concerns a difference between cells, e.g. it is equally feasible that the combination is effective when the cell surface levels of the membrane-bound protein in the second cell are decreased at least about 5%, 10%, 20%, 30%, 40%, 50%, 60%, 70%, 80%, 90%, 95% or about 100% as compared to the cell surface levels of the membrane-bound protein in the first cell after step b). In this scenario, the second cell thus comprises the effective combination.

Preferably a third, fourth or further cells are provided expressing respectively a third, a fourth or a further transmembrane E3 ubiquitin ligase and the first membrane-bound protein at their cell surface, wherein the transmembrane E3 ubiquitin ligases are different ligases comprising the same first extracellular non-native epitope tag, and wherein the combination is effective when the cell surface levels of the membrane-bound protein in the first cell are decreased at least about 2%, 5%, 10%, 20%, 30%, 40%, 50%, 60%, 70%, 80%, 90%, 95% or about 100% as compared to the cell surface levels of the membrane-bound protein in the second, third, fourth and further cells after step b). Preferably the method is performed in a multiplexed manner.

Using the method of the invention, the most effective combination can therefore be determined. The most effective combination is preferably the combination of a transmembrane E3 ubiquitin ligase and a membrane-bound protein, wherein the combination results in the strongest decrease in the cell surface levels of the membrane-bound protein upon simultaneous binding to the heterobifunctional molecule. Preferably, the most effective combination is the combination that results in a decrease in the cell surface levels of the membrane-bound protein of at least about 2%, 5%, 10%, 20%, 30%, 40%, 50%, 60%, 70%, 80%, 90% or at least about 95% as compared to all the other tested combinations. The most effective combination for a certain membrane-bound protein is preferably the transmembrane E3 ubiquitin ligase that most efficiently mediates removal of this membrane-bound protein from the cell surface, as compared to the other tested transmembrane E3 ubiquitin ligases, upon simultaneous binding of the transmembrane E3 ubiquitin ligase and the membrane-bound protein to the heterobifunctional molecule.

In addition, the skilled person straightforwardly understands that the method as detailed herein is not limited by these examples and variations are equally part of the present invention.

The protein level of the membrane-bound protein may be determined before and after exposure to the heterobifunctional molecule as defined herein.

The protein levels of the membrane-bound protein may be determined after exposing the cell to a heterobifunctional molecule for at least about 0.5, 1, 2, 3, 4, 5, 6, 7, 8, 9, 10, 11, 12, 14, 16, 18, 20, 22 or 24 hours, or preferably for at least 1, 2, 3, 4, 5, 6 or 7 days.

Preferably, the cell surface levels of the membrane-bound protein are decreased as compared to the cell surface levels of the membrane-bound protein of a same cell that is not exposed to the heterobifunctional molecule, e.g. the cell provided in step a) of the method of the invention.

The term “(protein) level” and “(protein) amount” may be used interchangeably herein. Preferably, the cell surface level is decreased at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or decreased about 100% as compared to the cell surface level of the membrane-bound protein prior to step b) of the method of the invention. It is understood herein that a decrease of 100% indicates that the transmembrane protein is no longer detectable on the surface of the cell. The decrease in cell surface protein levels may be determined by directly determining the level, or amount, of protein remaining on the surface of the cell after exposure to the heterobifunctional molecule. A preferred method for detecting the level of the membrane-bound protein on the cell surface level is immunofluorescence, preferably quantitative immunofluorescence.

The transmembrane ubiquitin E3 ligase preferably ubiquitinates the membrane-bound protein. Therefore alternatively or in addition, the decrease in surface level of the membrane-bound protein may be determined by determining the level of ubiquitination of the membrane-bound protein. An increase in ubiquitination levels is preferably reversely correlates with the cell surface level of the membrane-bound protein. Methods to determine the ability of a transmembrane E3 ubiquitin ligase to ubiquitinate a membrane-bound protein include, but are not limited to, immunoprecipitation of the membrane-bound protein, e.g. via the third epitope tag, and determination of the bound ubiquitin molecules using an anti-ubiquitin antibody. Alternatively or in addition, tagged, preferably His-tagged, ubiquitin can be co-expressed with the membrane-bound protein and levels of ubiquitination can be determined using anti-tag, preferably anti-His tag, antibodies. In addition or alternatively, proteomics approaches can be used to identify and quantify the ubiquitin chains (reviewed in Fulzele and Bennett 2018 Methods Mol Biol).

Ubiquitination preferably results in internalisation and degradation of the ubiquitinated membrane-bound protein. Therefore alternatively or in addition, the decrease in surface level of the membrane-bound protein may be determined by determining the cell's total protein level, or amount, of the membrane-bound protein after exposure to the heterobifunctional molecule, e.g. after step b). The cell's total protein level is preferably decreased at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or decreased about 100% as compared to the cell's total protein level of the membrane-bound protein prior to step b) of the method of the invention. It is understood herein that a decrease of 100% indicates that the transmembrane protein is no longer detectable in the cell. Methods for determining total protein levels are well known, and include e.g. standard biochemical analyses and FACS.

Alternatively or in addition, the decrease in cell surface levels of the membrane-bound protein may be determined by determining an increase in intracellular localization of the membrane-bound protein, preferably an increase in the endosomal localization of the membrane-bound protein. Ubiquitination of the membrane-bound protein preferably results in internalisation of the membrane-bound protein. The internalized protein may subsequently be degraded, preferably degraded in the lysosome. To determine the increase in intracellular protein localization of the protein, the method may comprise a step of inhibiting lysosomal turnover, prior to a step of determining the increase in intracellular localization of the membrane-bound protein, for example, but not limited to, by treating the cells with bafilomycin A1. Preferably the intracellular localization of the membrane-bound protein is increased at least about 1.5-, 2-, 3-, 4-, 5-, 6-fold or more as compared to the intracellular localization of the membrane-bound protein prior to step b) of the method of the invention.

Alternatively or in addition, reporter assays and/or downstream signalling read outs may be used to determine the decrease in the cell surface level of the membrane-bound protein. Such reporter assays and/or downstream signalling read outs are known in the art and are readily available for use in the method of the invention.

Alternatively or in addition, the co-localisation of the membrane-bound proteins and one or more lysosomal markers may be determined using standard technologies. The level of co-localisation preferably reversely correlates with the cell surface level of the membrane-bound protein.

The method as defined herein above may also be considered a method for selecting a combination of a transmembrane E3 ubiquitin ligase and a membrane-bound protein. Preferably, the method as defined herein is a method for selecting an effective combination of a transmembrane E3 ubiquitin ligase and a membrane-bound protein. Preferably the combination is an effective combination when the transmembrane E3 ubiquitin ligase is capable of ubiquitination of the membrane-bound protein when they are brought in close proximity. The ubiquitination of the membrane-bound protein preferably results in internalisation of said protein. Preferably, the transmembrane E3 ubiquitin ligase and the membrane-bound protein are brought in close proximity by simultaneous binding to a heterobifunctional molecule as defined herein.

The method of the invention as defined herein above may also be considered a method for determining the efficiency or potency of a heterobifunctional molecule, preferably a heterobifunctional molecule as defined herein, to decrease the surface level of a membrane-bound protein of a cell. The method preferably comprises the steps as outlined above. Preferably, the method comprises the steps of:
a) Providing the cell, wherein the cell expresses a transmembrane E3 ubiquitin ligase and the membrane-bound protein at its cell surface; and
b) Exposing the cell to a heterobifunctional molecule, preferably a heterobifunctional molecule as defined herein.
   The method preferably further comprises a step c) of determining the surface levels of the membrane-bound protein of the cell. The decrease is preferably a decrease as compared to the surface levels of the membrane-bound protein of the cell prior to step b).

The method as defined herein may also be considered at least one of:
a method for selecting an effective combination of a transmembrane E3 ubiquitin ligase and a membrane-bound protein, preferably a transmembrane protein, and wherein the combination is selected when the protein levels of the transmembrane protein are decreased after step c).
a method for screening for an effective combination of a transmembrane E3 ubiquitin ligase and a membrane-bound protein, preferably a transmembrane protein;
a method for manufacturing a heterobifunctional molecule as defined herein, wherein the heterobifunctional molecule selectively binds to a selected combination of a transmembrane E3 ubiquitin ligase and a membrane-bound protein, preferably a transmembrane protein;
a method for determining the ability of a transmembrane E3 ubiquitin ligase to ubiquitinate a membrane-bound protein, wherein the transmembrane E3 ligase is able to ubiquitinate the membrane-bound protein when the protein levels of the transmembrane protein are decreased after step c);
a method for targeting a membrane-bound protein for degradation by a heterobifunctional molecule; and
a method for determining the ubiquitination of a membrane-bound protein, and wherein a decrease in the surface levels of the membrane-bound protein indicates ubiquitination of the membrane-bound protein, wherein the decrease is preferably a decrease as compared to the surface levels of the membrane-bound protein of the cell prior to step b).

As indicated herein above, the method as defined herein may be used to manufacture an effective heterobifunctional molecule, e.g. a heterobifunctional molecule that targets an effective combination of a transmembrane E3 ubiquitin ligase and a membrane-bound protein. The invention therefore also pertains to a heterobifunctional molecule, preferably a heterobifunctional molecule as defined herein, wherein the transmembrane E3 ubiquitin ligase and the membrane-bound protein, that are selectively bound by said heterobifunctional molecule, are selected using a method, preferably a selection method, as defined herein above. The method thus preferably comprises the steps of:
a) Providing a cell expressing a transmembrane E3 ubiquitin ligase and a membrane-bound protein at its cell surface, and wherein
   the transmembrane E3 ubiquitin ligase comprises a first non-native epitope tag in the extracellular portion; and
   the membrane-bound protein comprises a second non-native epitope tag in the extracellular portion;
b) Exposing the cell to a heterobifunctional molecule, wherein the heterobifunctional molecule comprises:
   a first binding domain capable of specific binding to the first non-native epitope tag; and
   a second binding domain capable of binding to the second non-native epitope tag;
c) determining the surface levels of the membrane-bound protein of the cell; and
d) selecting the transmembrane E3 ubiquitin ligase and the transmembrane protein when the surface levels of the membrane-bound protein are decreased at least about 10%, 20%, 30%, 40%, 50%, 60%, 70%, 80%, 90% or about 100%, and wherein the decrease is a decrease as compared to the surface levels of the membrane-bound protein of the cell prior to step b).

In an aspect, the invention pertains to a transmembrane E3 ubiquitin ligase comprising a first, and optional fourth, non-native epitope tag as defined herein.

In another aspect, the invention pertains to a membrane-bound protein comprising a second, and optional third, non-native epitope tag as defined herein.

In an aspect, the invention pertains to a combination of
a transmembrane E3 ubiquitin ligase comprising a first, and optional fourth, non-native epitope tag as defined herein; and
a membrane bound protein comprising a second, and optional third, non-native epitope tag as defined herein.

In an aspect, the invention pertains to a host cell expressing the transmembrane E3 ubiquitin ligase comprising a first, and optional fourth, non-native epitope tag as defined herein. The host cell preferably may further express a membrane-bound protein comprising a second, and optional third, non-native epitope tag as defined herein. A preferred host cell of the invention is a cell provided in step a) the method of the invention.

### Transmembrane E3 Ubiquitin Ligase Bound by a First Binding Domain

The invention further pertains to a heterobifunctional molecule for use in the method of the invention. In addition, the invention concerns a heterobifunctional molecule targeting an effective combination of a transmembrane E3 ubiquitin ligase and a membrane-bound protein, preferably as identified by the method of the invention.

The heterobifunctional molecule of the invention comprises a first binding domain capable of specific binding to an extracellular portion of the transmembrane E3 ubiquitin ligase and a second binding domain capable of specific binding to an extracellular portion of the membrane-bound protein.

The first binding domain of the heterobifunctional molecule is capable of specific binding to a transmembrane E3 ubiquitin ligase. Preferably, the transmembrane E3 ubiquitin ligase comprises a native epitope that can be specifically bound by a heterobifunctional molecule as defined herein, preferably when the heterobifunctional molecule is for use as a medicament. Alternatively, the transmembrane E3 ubiquitin ligase may be engineered to comprise a non-native epitope, preferably a non-native epitope as defined herein. The non-native epitope may be specifically bound by the heterobifunctional molecule, preferably when the heterobifunctional molecule is used in a method of the invention, preferably a selection method of the invention.

The transmembrane E3 ubiquitin ligase may mediate ubiquitination and endocytosis of a membrane-bound protein, i.e. can mediate ubiquitination and endocytosis of the protein bound by the second binding domain of the heterobifunctional molecule as defined herein.

Ubiquitination and endocytosis of the substrate preferably leads to removal of the substrate from the cell surface. The internalised substrate may subsequently be degraded. Hence, preferably the transmembrane E3 ubiquitin ligase may mediate ubiquitination, cell surface removal and degradation of a membrane-bound protein, i.e. may mediate ubiquitination, cell surface removal and degradation of the protein bound by the second binding domain of the heterobifunctional molecule as defined herein.

Hence preferably, simultaneous binding of the heterobifunctional molecule to the transmembrane E3 ubiquitin ligase and the membrane-bound protein results in internalisation of the membrane-bound protein, thereby removing the membrane-bound protein from the cell surface.

Preferably, simultaneous binding of the heterobifunctional molecule to the transmembrane E3 ubiquitin ligase and the membrane-bound protein results in internalisation and degradation of the membrane-bound protein. Therefore preferably, the transmembrane E3 ubiquitin ligase and the membrane-bound protein are expressed in the same cell. Optionally, at least one of the transmembrane E3 ubiquitin ligase and the membrane-bound protein may be overexpressed in the cell.

Ubiquitination and degradation can be assessed using any suitable method known in the art. As a non-limiting example, ubiquitination and degradation can be assessed as described in Koo et al, Nature (2012), supra, which is incorporated herein by reference.

Substrate proteins are selected for modification of lysine residues by ubiquitin through interaction with an E3 ligase protein that recruits an E2-enzyme charged with ubiquitin (Clague M J and Urbé S (2010), Cell; 43(5):682-5). This can result in transfer of a single ubiquitin molecule (monoubiquitination) to the substrate or coupling of a further ubiquitin molecule to the previous ubiquitin molecule, e.g. through lysine residues present in the previous ubiquitin molecule, to form a chain. The seven lysines of ubiquitin provide for the formation of different isopeptide chain linkages, which adopt different three-dimensional structures, and all of which are represented in eukaryotic cells (Xu et al. (2009), Cell 137, 133-145). The specific combination of E2 and E3 enzymes recruited to a substrate dictates the chain linkage type.

Notably, lysosomal degradation may require a different ubiquitination pattern of the substrate protein than proteasomal degradation. For example, a substrate tagged with a lysine 48 (Lys48)-linked polyubiquitin chain often results in proteasomal targeting. Alternatively, substrates tagged with either mono-ubiquitin, multi-ubiquitin, Lys11-, Lys29-, Lys48-linked or Lys63-linked polyubiquitin are directed to the lysosome.

The degradation mediated by the transmembrane E3 ubiquitin ligase may be at least one of lysosomal degradation and proteasomal degradation. Preferably, the degradation mediated by the transmembrane E3 ubiquitin ligase is at least lysosomal degradation.

Hence, preferably simultaneous binding of the heterobifunctional molecule to the transmembrane E3 ubiquitin ligase and the membrane-bound protein results in internalization of the membrane-bound protein. Preferably simultaneous binding of the heterobifunctional molecule to the transmembrane E3 ubiquitin ligase and the membrane-bound protein results in internalization, and at least one of proteasomal and lysosomal degradation of the membrane-bound protein. Preferably simultaneous binding of the heterobifunctional molecule to the transmembrane E3 ubiquitin ligase and the membrane-bound protein results in internalization and lysosomal degradation of the membrane-bound protein.

Preferably, the transmembrane E3 ubiquitin ligase ubiquitinates the membrane-bound protein with monoubiquitin, multiubiquitin, Lys11-, Lys29-, Lys48- or Lys63-linked polyubiquitin chains. Preferably, the transmembrane E3 ubiquitin ligase polyubiquitinates the membrane-bound protein with monoubiquitin, multiubiquitin, or Lys63-linked polyubiquitin chains. Preferably, the transmembrane E3 ubiquitin ligase polyubiquitinates the membrane-bound protein with at least one of Lys11-, Lys29-, Lys48- and Lys63-linked polyubiquitin chains. Preferably, the transmembrane E3 ubiquitin ligase polyubiquitinates the membrane-bound protein with Lys63-linked polyubiquitin chains.

A number of transmembrane E3 ubiquitin ligases display tissue-specific expression or show overexpression in one or more cancer types. Hence preferably the transmembrane E3 ubiquitin ligase that can be bound by a heterobifunctional molecule as defined herein, is a transmembrane E3 ubiquitin ligase that is expressed in a selective tissue. As a non-limiting example, the transmembrane E3 ubiquitin ligases RNF43 and ZNRF3 are selectively expressed in the adult stem cell population of multiple tissues, such as, but not limited to, the intestine. As a further non-limited example, the transmembrane E3 ubiquitin ligases MARCH1 and MARCH9 show increased expression in immune cells.

Preferably the transmembrane E3 ubiquitin ligase is only expressed in a selective tissue, such as but not limited to, a cancerous tissue.

Alternatively or in addition, the transmembrane E3 ubiquitin ligase that can be bound by a heterobifunctional molecule as defined herein, is a transmembrane E3 ubiquitin ligase that shows expression, preferably overexpression, in one or more types of cancer.

Preferably, the transmembrane E3 ubiquitin ligase is selected from the group consisting of RNF43, RNF167, ZNRF3, RNF13, AMFR, MARCH1, MARCH2, MARCH4, MARCH8, MARCH9, RNF149, RNF145, RNFT1, RNF130 and RNF128. Preferably, the transmembrane E3 ubiquitin ligase is selected from the group consisting of RNF43, RNF167, ZNRF3, RNF13, AMFR, MARCH1, MARCH2, MARCH4, MARCH8, MARCH9, RNF145, RNFT1, RNF130 and RNF128. Preferably, the transmembrane E3 ubiquitin ligase is at least one of RNF43, RNF167, RNF128 and RNF130. Preferably, the transmembrane E3 ubiquitin ligase is at least one of RNF43, RNF128 and RNF167. Preferably, the transmembrane E3 ubiquitin ligase is at least one of RNF43 and RNF128.

The transmembrane E3 ubiquitin ligase may be overexpressed. As a non-limiting example, it is known in the art that RNF43, ZNRF3, RNF13, AMFR, MARCH1, MARCH2, MARCH4, MARCH8, MARCH9, RNF149, RNF145, RNFT1, RNF167, RNF130 and RNF128 show an increased expression in cancer.

In an embodiment, the heterobifunctional molecule comprises a first and a second binding domain, wherein

i) the first binding domain is capable of specific binding to a transmembrane E3 ubiquitin ligase, wherein the transmembrane E3 ubiquitin ligase is expressed, preferably selectively expressed, or overexpressed in a cancerous tissue; and

ii) the second binding domain is capable of specific binding to a membrane-bound protein, wherein the membrane-bound protein is known or expected to be involved in said cancerous tissue,

wherein simultaneous binding of the heterobifunctional molecule to the transmembrane E3 ubiquitin ligase and the membrane-bound protein preferably results in ubiquitination and internalisation of the transmembrane protein.

Preferably, the transmembrane E3 ubiquitin ligase and the membrane-bound protein are expressed in the same cell, preferably are expressed in the same cancerous cell.

Preferably, the E3 ubiquitin ligase and the membrane-bound protein are both expressed in a cancerous cell selected from the group consisting of lung cancer, colorectal cancer, hepatocellular carcinoma, osteosarcoma, pancreatic cancer, gastric cancer, liver cancer, skin cancer, breast cancer, bladder cancer, ovarian cancer, esophageal cancer, thyroid cancer, cervical cancer, glioblastoma, squamous cell carcinoma, prostate cancer (Gene expression atlas) and intestinal cancer and/or metastases thereof.

Preferably the membrane-bound protein is a transmembrane protein.

In an embodiment, the heterobifunctional molecule comprises a first and a second binding domain, wherein

i) the first binding domain is capable of specific binding to a transmembrane E3 ubiquitin ligase, wherein the transmembrane E3 ubiquitin ligase is expressed, preferably selectively expressed, or overexpressed in an immune cell; and

ii) the second binding domain is capable of specific binding to a membrane-bound protein, wherein the membrane-bound protein is expressed in the same immune cell, wherein simultaneous binding of the heterobifunctional molecule to the transmembrane E3 ubiquitin ligase and the membrane-bound protein preferably results in ubiquitination and internalisation of the membrane-bound protein.

Preferably the membrane-bound protein is a transmembrane protein.

In an embodiment, the heterobifunctional molecule comprises a first and a second binding domain, wherein

i) the first binding domain is capable of specific binding to a transmembrane E3 ubiquitin ligase, wherein the transmembrane E3 ubiquitin ligase is expressed, preferably selectively expressed, or overexpressed in a neural cell; and

ii) the second binding domain is capable of specific binding to a membrane-bound protein, wherein the membrane-bound protein is expressed in the same neural cell, wherein simultaneous binding of the heterobifunctional molecule to the transmembrane E3 ubiquitin ligase and the membrane-bound protein preferably results in ubiquitination and internalisation of the membrane-bound protein.

Preferably the membrane-bound protein is a transmembrane protein.

Preferably, the transmembrane E3 ubiquitin ligase has at least about 60%, 65%, 70%, 75%, 80%, 85%, 90%, 95%, 96%, 97%, 98%, 99% or 100% sequence identity with a sequence selected from the group consisting of SEQ ID NO: 1, 3, 5, 7, 9, 11, 13, 15, 17, 19, 21, 23, 25, 27 and 29.

Preferably, the transmembrane E3 ubiquitin ligase is encoded by a sequence having at least about 60%, 65%, 70%, 75%, 80%, 85%, 90%, 95%, 96%, 97%, 98%, 99% or 100% sequence identity with a sequence selected from the group consisting of SEQ ID NO: 2, 4, 6, 8, 10, 12, 14, 16, 18, 20, 22, 24, 26, 28 and 30.

Preferably, the transmembrane E3 ubiquitin ligase is at least one of RNF43 and ZNRF3. The proteins RNF43 and ZNRF3 preferably have at least about 60%, 65%, 70%, 75%, 80%, 85%, 90%, 95%, 96%, 97%, 98%, 99% or 100% sequence identity with respectively SEQ ID NO: 1 and 3. Preferably, the RNF43 and ZNRF3 proteins are encoded by a sequence having at least about 60%, 65%, 70%, 75%, 80%, 85%, 90%, 95%, 96%, 97%, 98%, 99% or 100% sequence identity with respectively SEQ ID NO: 2 and 4.

In preferred embodiments, the transmembrane E3 ubiquitin ligase is RNF43. Preferably, the RNF43 protein has at least about 60%, 65%, 70%, 75%, 80%, 85%, 90%, 95%, 96%, 97%, 98%, 99% or 100% sequence identity with SEQ ID NO: 1. Preferably, the RNF43 protein is encoded by a sequence having at least about 60%, 65%, 70%, 75%, 80%, 85%, 90%, 95%, 96%, 97%, 98%, 99% or 100% sequence identity with SEQ ID NO: 2.

In preferred embodiments, the transmembrane E3 ubiquitin ligase is ZNRF3. Preferably, the ZNRF3 protein has at least about 60%, 65%, 70%, 75%, 80%, 85%, 90%, 95%, 96%, 97%, 98%, 99% or 100% sequence identity with SEQ ID NO: 3. Preferably, the ZNRF3 protein is encoded by a sequence having at least about 60%, 65%, 70%, 75%, 80%, 85%, 90%, 95%, 96%, 97%, 98%, 99% or 100% sequence identity with SEQ ID NO: 4.

In preferred embodiments, the transmembrane E3 ubiquitin ligase is RNF13. Preferably, the RNF13 protein has at least about 60%, 65%, 70%, 75%, 80%, 85%, 90%, 95%, 96%, 97%, 98%, 99% or 100% sequence identity with SEQ ID NO: 5. Preferably, the RNF13 protein is encoded by a sequence having at least about 60%, 65%, 70%, 75%, 80%, 85%, 90%, 95%, 96%, 97%, 98%, 99% or 100% sequence identity with SEQ ID NO: 6.

In preferred embodiments, the transmembrane E3 ubiquitin ligase is AMFR. Preferably, the AMFR protein has at least about 60%, 65%, 70%, 75%, 80%, 85%, 90%, 95%, 96%, 97%, 98%, 99% or 100% sequence identity with SEQ ID NO: 7 (or SEQ ID NO: 51). Preferably, the AMFR protein is encoded by a sequence having at least about 60%, 65%, 70%, 75%, 80%, 85%, 90%, 95%, 96%, 97%, 98%, 99% or 100% sequence identity with SEQ ID NO: 8 (or SEQ ID NO: 52).

In preferred embodiments, the transmembrane E3 ubiquitin ligase is MARCH1. Preferably, the MARCH1 protein has at least about 60%, 65%, 70%, 75%, 80%, 85%, 90%, 95%, 96%, 97%, 98%, 99% or 100% sequence identity with SEQ ID NO: 9. Preferably, the MARCH1 protein is encoded by a sequence having at least about 60%, 65%, 70%, 75%, 80%, 85%, 90%, 95%, 96%, 97%, 98%, 99% or 100% sequence identity with SEQ ID NO: 10.

In preferred embodiments, the transmembrane E3 ubiquitin ligase is MARCH4. Preferably, the MARCH4 protein has at least about 60%, 65%, 70%, 75%, 80%, 85%, 90%, 95%, 96%, 97%, 98%, 99% or 100% sequence identity with SEQ ID NO: 11. Preferably, the MARCH4 protein is encoded by a sequence having at least about 60%, 65%, 70%, 75%, 80%, 85%, 90%, 95%, 96%, 97%, 98%, 99% or 100% sequence identity with SEQ ID NO: 12.

In preferred embodiments, the transmembrane E3 ubiquitin ligase is MARCH2. Preferably, the MARCH2 protein has at least about 60%, 65%, 70%, 75%, 80%, 85%, 90%, 95%, 96%, 97%, 98%, 99% or 100% sequence identity with SEQ ID NO: 13. Preferably, the MARCH2 protein is encoded by a sequence having at least about 60%, 65%, 70%, 75%, 80%, 85%, 90%, 95%, 96%, 97%, 98%, 99% or 100% sequence identity with SEQ ID NO: 14.

In preferred embodiments, the transmembrane E3 ubiquitin ligase is MARCH8. Preferably, the MARCH8 protein has at least about 60%, 65%, 70%, 75%, 80%, 85%, 90%, 95%, 96%, 97%, 98%, 99% or 100% sequence identity with SEQ ID NO: 15. Preferably, the MARCH8 protein is encoded by a sequence having at least about 60%, 65%, 70%, 75%, 80%, 85%, 90%, 95%, 96%, 97%, 98%, 99% or 100% sequence identity with SEQ ID NO: 16.

In preferred embodiments, the transmembrane E3 ubiquitin ligase is MARCH9. Preferably, the MARCH9 protein has at least about 60%, 65%, 70%, 75%, 80%, 85%, 90%, 95%, 96%, 97%, 98%, 99% or 100% sequence identity with SEQ ID NO: 17. Preferably, the MARCH9 protein is encoded by a sequence having at least about 60%, 65%, 70%, 75%, 80%, 85%, 90%, 95%, 96%, 97%, 98%, 99% or 100% sequence identity with SEQ ID NO: 18.

In preferred embodiments, the transmembrane E3 ubiquitin ligase is RNF149. Preferably, the RNF149 protein has at least about 60%, 65%, 70%, 75%, 80%, 85%, 90%, 95%, 96%, 97%, 98%, 99% or 100% sequence identity with SEQ ID NO: 19. Preferably, the RNF149 protein is encoded by a sequence having at least about 60%, 65%, 70%, 75%, 80%, 85%, 90%, 95%, 96%, 97%, 98%, 99% or 100% sequence identity with SEQ ID NO: 20.

In preferred embodiments, the transmembrane E3 ubiquitin ligase is RNF145. Preferably, the RNF145 protein has at least about 60%, 65%, 70%, 75%, 80%, 85%, 90%, 95%, 96%, 97%, 98%, 99% or 100% sequence identity with SEQ ID NO: 21. Preferably, the RNF145 protein is encoded by a sequence having at least about 60%, 65%, 70%, 75%, 80%, 85%, 90%, 95%, 96%, 97%, 98%, 99% or 100% sequence identity with SEQ ID NO: 22.

In preferred embodiments, the transmembrane E3 ubiquitin ligase is RNFT1. Preferably, the RNFT1 protein has at least about 60%, 65%, 70%, 75%, 80%, 85%, 90%, 95%, 96%, 97%, 98%, 99% or 100% sequence identity with SEQ ID NO: 23. Preferably, the RNFT1 protein is encoded by a sequence having at least about 60%, 65%, 70%, 75%, 80%, 85%, 90%, 95%, 96%, 97%, 98%, 99% or 100% sequence identity with SEQ ID NO: 24.

In preferred embodiments, the transmembrane E3 ubiquitin ligase is RNF167. Preferably, the RNF167 protein has at least about 60%, 65%, 70%, 75%, 80%, 85%, 90%, 95%, 96%, 97%, 98%, 99% or 100% sequence identity with SEQ ID NO: 25. Preferably, the RNF167 protein is encoded by a sequence having at least about 60%, 65%, 70%, 75%, 80%, 85%, 90%, 95%, 96%, 97%, 98%, 99% or 100% sequence identity with SEQ ID NO: 26.

In preferred embodiments, the transmembrane E3 ubiquitin ligase is RNF130. Preferably, the RNF130 protein has at least about 60%, 65%, 70%, 75%, 80%, 85%, 90%, 95%, 96%, 97%, 98%, 99% or 100% sequence identity with SEQ ID NO: 27. Preferably, the RNF130 protein is encoded by a sequence having at least about 60%, 65%, 70%, 75%, 80%, 85%, 90%, 95%, 96%, 97%, 98%, 99% or 100% sequence identity with SEQ ID NO: 28.

In preferred embodiments, the transmembrane E3 ubiquitin ligase is RNF128. Preferably, the RNF128 protein has at least about 60%, 65%, 70%, 75%, 80%, 85%, 90%, 95%, 96%, 97%, 98%, 99% or 100% sequence identity with SEQ ID NO: 29. Preferably, the RNF128 protein is encoded by a sequence having at least about 60%, 65%, 70%, 75%, 80%, 85%, 90%, 95%, 96%, 97%, 98%, 99% or 100% sequence identity with SEQ ID NO: 30.

### Protein Bound by the Second Binding Domain

As detailed herein, the heterobifunctional molecule of the invention has a first binding domain capable of binding a transmembrane E3 ubiquitin ligase, preferably a transmembrane E3 ubiquitin ligase as defined herein above.

The heterobifunctional molecule of the invention further comprises a second binding domain, wherein the second binding domain is capable of binding a membrane-bound protein. Preferably, the membrane-bound protein comprises a native epitope that can be specifically bound by a heterobifunctional molecule as defined herein, preferably when the heterobifunctional molecule is for use as a medicament. Alternatively, the membrane-bound protein may be engineered to comprise a non-native epitope, preferably a non-native epitope as defined herein. The non-native epitope may be specifically bound by the heterobifunctional molecule, preferably when the heterobifunctional molecule is used in a method of the invention, preferably a selection method of the invention.

Preferably, the protein that can be bound by the second binding domain of the heterobifunctional molecule is a protein that is at least partly exposed to the exterior of the cell. The protein may be attached to the cell membrane from one side or may span the entirety of the membrane, i.e. is a transmembrane protein. Preferably, the second binding domain is capable of specific binding to a transmembrane protein.

As the term “heterobifunctional” already suggests, the membrane-bound protein that can be bound by the second binding domain is distinct from the transmembrane E3 ubiquitin ligase that can be bound by the first binding domain. Preferably, the second binding domain does not specifically and/or effectively bind any transmembrane E3 ubiquitin ligase.

Preferably, the membrane-bound protein is a transmembrane protein, preferably at least one of a nutrient transporter, an ion channel and a cell surface receptor. Preferably the second binding domain of the heterobifunctional molecules is capable of specific binding to a transmembrane receptor. Preferably, the receptor is at least one of an ion channel-linked receptor, an enzyme-linked receptor, a G protein-coupled receptor and an Fc Receptor. The second binding domain of the heterobifunctional molecule may bind to the monomeric form and/or the dimerized form of a receptor. In addition or alternatively, the second binding domain may bind to the inactive and/or active conformation of a receptor.

The membrane-bound protein may be associated with, or involved in, the development, progression or severity of a disease. The membrane-bound protein may be known or expected to be involved in a cancer, an auto-immune disease, an inflammatory disease, a neurological disorder, a rare disease, an infectious disease and/or in a hereditary disease.

Preferably, the membrane-bound protein is not at least one of LGR4, LGR5 and LGR6.

In preferred embodiments, the membrane-bound protein is known or expected to be involved in a neurological disorder. In preferred embodiments, the membrane-bound protein is known or expected to be involved in a rare disease.

In preferred embodiments, the membrane-bound protein is known or expected to be involved in a disease selected from the group consisting of Charcot Marie Tooth Disease (CMT), Gaucher Disease (GD), Anti-Mag peripheral Neuropathy, CD38 associated neurodegenerative pathology, Myostatin associated neuromuscular disease, demyelating disease, MS, ALS and Gullain-Barre (GB). The CD38 associated neurodegenerative pathology may at least one of ALS, MS, PD and AD. The Myostatin associated neuromuscular disease may be at least one of Duschenne's and cachexia.

In preferred embodiments, the membrane-bound protein, preferably the membrane-bound receptor, is known or expected to be involved in cancer. “A receptor involved in cancer” is herein understood as a membrane-bound receptor which can directly or indirectly influence the malignancy of a cancer.

In an embodiment, the membrane-bound receptor involved in cancer can be a receptor which, upon activation or increased activity, induces or augments malignant properties of a cell. For example, but not limited to, activation of the membrane-bound receptor may have an impact on at least one of the stemness, differentiation capacity, metabolism, viability, proliferation and immune evasive capacity of a cell. Activation of a receptor as used herein includes, but is not limited to, a receptor having one or more activating mutations and/or a receptor having an increased expression and/or an increased availability of the receptor ligand and/or receptors having a decreased turnover, e.g. are stabilized on the cell membrane.

In addition or alternatively, the membrane-bound receptor known or expected to be involved in cancer can be a receptor present on e.g. immune cells and/or stromal cells. As a non-limiting example, inhibiting a receptor present on an immune cell can result in the activation of the immune cell to target the tumor cells and the inhibition of a receptor present on stromal cells can result in reduced tumor angiogenesis.

Hence, it is understood herein that the receptor involved in cancer can be a membrane-bound receptor present on a tumor cell, and/or a membrane-bound receptor present on a cell that has an, direct or indirect, effect on the tumor cell.

The phrase “receptor associated with or involved in cancer” includes, but is not limited to, proliferative diseases such as a cancer or malignancy or a precancerous condition such as a myelodysplasia, a myelodysplastic syndrome or a preleukemia.

In one embodiment, a cancer associated with the activation or increased activity of membrane-bound receptor as described herein is a hematological cancer. In one embodiment, a cancer associated with activation or increased activity of a membrane-bound receptor as described herein is a solid cancer. Further diseases associated with the activation or increased activity of a membrane-bound receptor as described herein include, but not is limited to, e.g., atypical and/or non-classical cancers, malignancies, precancerous conditions or proliferative diseases associated with the activation of a membrane-bound receptor as described herein. Non-cancer related indications associated with the activation or increased activity of a membrane-bound receptor as described herein include, but are not limited to, e.g., autoimmune disease, (e.g., lupus), inflammatory disorders (allergy and asthma) and transplantation.

Preferably a membrane-bound receptor that can be bound by the second domain of the heterobifunctional molecule is a receptor involved in cancer, whereby preferably the receptor has an increased activity, e.g. an increased downstream signalling. The downstream signalling is preferably increased as compared to an otherwise identical cell that does not have an activation or increased activity of the membrane-bound receptor. The increased activity may be due to, but not limited to, mutational activation of the receptor, upregulation of the receptor, an increased stabilization of the receptor and/or an increased availability of the receptor ligand.

The receptor may be involved in one specific type of cancer. Alternatively, the receptor may play be involved in many different cancer types. For example, the receptor may be involved in at least 1, 2, 3, 4, 5, 6, 7, 8, 9, 10 or more types of cancer. Alternatively or in addition, the receptor may be involved in cancer angiogenesis.

The receptor may be involved in a solid cancer or a hematologic cancer. The receptor may be involved in a solid cancer. Preferably, the solid cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

The receptor may be involved in a solid cancer or a hematologic cancer. Preferably, the hematologic cancer is chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia.

Preferably, the membrane-bound protein that can be bound by the second binding domain of the heterobifunctional molecule is involved in a cancer selected from the group consisting of colorectal cancer, ovarian cancer, breast cancer, esophagal cancer, gastric cancer, prostate cancer, lung cancer, melanoma, leukemia, pancreatic cancer and bladder cancer.

Preferably, the membrane-bound protein that can be bound by the second binding domain of the heterobifunctional molecule is activated or has an increased activity in colorectal cancer. Preferably, the membrane-bound protein is at least one of EGFR, IGF1R, MET, LRP6, WLS and ERBB2.

Preferably, the transmembrane protein that can be bound by the second binding domain of the heterobifunctional molecule is activated or has an increased activity in hepatocellular cancer. Preferably, the membrane-bound protein is LRP6.

Preferably, the transmembrane protein that can be bound by the second binding domain of the heterobifunctional molecule is activated or has an increased activity in colorectal cancer. Preferably, the membrane-bound protein is LRP6 or WLS.

Preferably, the transmembrane protein that can be bound by the second binding domain of the heterobifunctional molecule is activated or has an increased activity in breast cancer. Preferably, the membrane-bound protein is WLS, EGFR or ERBB2.

Preferably, the transmembrane protein that can be bound by the second binding domain of the heterobifunctional molecule is activated or has an increased activity in oesophageal cancer. Preferably, the membrane-bound protein is ERBB2 or VEGFR2.

Preferably, the transmembrane protein that can be bound by the second binding domain of the heterobifunctional molecule is activated or has an increased activity in gastric cancer. Preferably, the membrane-bound protein is WLS, ERBB2 or VEGFR2.

Preferably, the transmembrane protein that can be bound by the second binding domain of the heterobifunctional molecule is activated or has an increased activity in leukaemia. Preferably, the membrane-bound protein is FLT3.

Preferably, the transmembrane protein that can be bound by the second binding domain of the heterobifunctional molecule is activated or has an increased activity in melanoma. Preferably, the membrane-bound protein is KIT.

Preferably, the transmembrane protein that can be bound by the second binding domain of the heterobifunctional molecule is activated or has an increased activity in non-small cell lung cancer. Preferably, the membrane-bound protein is EGFR or MET.

Preferably, the transmembrane protein that can be bound by the second binding domain of the heterobifunctional molecule is activated or has an increased activity in ovarian cancer. Preferably, the membrane-bound protein is EGFR.

Preferably, the transmembrane protein that can be bound by the second binding domain of the heterobifunctional molecule is activated or has an increased activity in pancreatic cancer. Preferably, the membrane-bound protein is LRP6 or EGFR.

Preferably, the membrane-bound protein that can be bound by the second binding domain of the heterobifunctional molecule is selected from the group consisting of TGFβR1, TGFβR2, EGFR, ERBB2, ERBB3, IGF1R, MET, VEGFR2, KIT, FLT3, PDGFRA, PDGFRB, GHR, FZD1, FZD2, FZD3, FZD4, FZD5, FZD6, FZD7, FZD8, FZD9, FZD10, LRP5, LRP6, PD-1, PD-L1, CTLA4, CMTM6, CMTM4, WLS, SLC7A5, and SLC16A7.

Preferably, the membrane-bound protein that can be bound by the second binding domain of the heterobifunctional molecule has at least about 60%, 65%, 70%, 75%, 80%, 85%, 90%, 95%, 96%, 97%, 98%, 99% or 100% sequence identity with a sequence selected from the group consisting of SEQ ID NO: 31, 33, 35, 37, 39, 41, 43, 45, 47, 49, 53, 55, 57, 59, 61, 63, 65, 67, 69, 71, 73, 75, 77, 79, 84, 86, 88, 90, 92, 94, 100, 102 and 104.

Preferably, the membrane-bound protein that can be bound by the second binding domain of the heterobifunctional molecule is encoded by a sequence having at least about 60%, 65%, 70%, 75%, 80%, 85%, 90%, 95%, 96%, 97%, 98%, 99% or 100% sequence identity with a sequence selected from the group consisting of SEQ ID NO: 32, 34, 36, 38, 40, 42, 44, 46, 48, 50, 54, 56, 58, 60, 62, 64, 66, 68, 70, 72, 74, 76, 78, 80, 85, 87, 89, 91, 93, 95, 101, 103 and 105.

In preferred embodiments, the transmembrane protein that can be bound by the second binding domain of the heterobifunctional molecule is TGFβR1 or TGFβR2.

In preferred embodiments, the transmembrane protein that can be bound by the second binding domain of the heterobifunctional molecule is TGFβR1. Preferably, the TGFβR1 protein has at least about 60%, 65%, 70%, 75%, 80%, 85%, 90%, 95%, 96%, 97%, 98%, 99% or 100% sequence identity with SEQ ID NO: 31. Preferably, the TGFβR1 protein is encoded by a sequence having at least about 60%, 65%, 70%, 75%, 80%, 85%, 90%, 95%, 96%, 97%, 98%, 99% or 100% sequence identity with SEQ ID NO: 32.

In preferred embodiments, the transmembrane protein that can be bound by the second binding domain of the heterobifunctional molecule is TGFβR2. Preferably, the TGFβR2 protein has at least about 60%, 65%, 70%, 75%, 80%, 85%, 90%, 95%, 96%, 97%, 98%, 99% or 100% sequence identity with SEQ ID NO: 33. Preferably, the TGFβR2 protein is encoded by a sequence having at least about 60%, 65%, 70%, 75%, 80%, 85%, 90%, 95%, 96%, 97%, 98%, 99% or 100% sequence identity with SEQ ID NO: 34.

Preferably, the second binding domain of the heterobifunctional molecule is capable of specific binding to TGFβR2, and the first binding domain is capable of specific binding to RNF167.

In preferred embodiments, the transmembrane protein that can be bound by the second binding domain of the heterobifunctional molecule is EGFR. Preferably, the EGFR protein has at least about 60%, 65%, 70%, 75%, 80%, 85%, 90%, 95%, 96%, 97%, 98%, 99% or 100% sequence identity with SEQ ID NO: 35. Preferably, the EGFR protein is encoded by a sequence having at least about 60%, 65%, 70%, 75%, 80%, 85%, 90%, 95%, 96%, 97%, 98%, 99% or 100% sequence identity with SEQ ID NO: 36.

Preferably, the second binding domain of the heterobifunctional molecule is capable of specific binding to EGFR, and the first binding domain is capable of specific binding to RNF167.

In preferred embodiments, the transmembrane protein that can be bound by the second binding domain of the heterobifunctional molecule is ERBB2 or ERBB3.

In preferred embodiments, the transmembrane protein that can be bound by the second binding domain of the heterobifunctional molecule is ERBB2. Preferably, the ERBB2 protein has at least about 60%, 65%, 70%, 75%, 80%, 85%, 90%, 95%, 96%, 97%, 98%, 99% or 100% sequence identity with SEQ ID NO: 37. Preferably, the ERBB2 protein is encoded by a sequence having at least about 60%, 65%, 70%, 75%, 80%, 85%, 90%, 95%, 96%, 97%, 98%, 99% or 100% sequence identity with SEQ ID NO: 38.

In preferred embodiments, the transmembrane protein that can be bound by the second binding domain of the heterobifunctional molecule is ERBB3. Preferably, the ERBB3 protein has at least about 60%, 65%, 70%, 75%, 80%, 85%, 90%, 95%, 96%, 97%, 98%, 99% or 100% sequence identity with SEQ ID NO: 39. Preferably, the ERBB3 protein is encoded by a sequence having at least about 60%, 65%, 70%, 75%, 80%, 85%, 90%, 95%, 96%, 97%, 98%, 99% or 100% sequence identity with SEQ ID NO: 40.

In preferred embodiments, the transmembrane protein that can be bound by the second binding domain of the heterobifunctional molecule is IGF1R. Preferably, the IGF1R protein has at least about 60%, 65%, 70%, 75%, 80%, 85%, 90%, 95%, 96%, 97%, 98%, 99% or 100% sequence identity with SEQ ID NO: 41. Preferably, the IGF1R protein is encoded by a sequence having at least about 60%, 65%, 70%, 75%, 80%, 85%, 90%, 95%, 96%, 97%, 98%, 99% or 100% sequence identity with SEQ ID NO: 42.

In preferred embodiments, the transmembrane protein that can be bound by the second binding domain of the heterobifunctional molecule is MET. Preferably, the MET protein has at least about 60%, 65%, 70%, 75%, 80%, 85%, 90%, 95%, 96%, 97%, 98%, 99% or 100% sequence identity with SEQ ID NO: 43. Preferably, the MET protein is encoded by a sequence having at least about 60%, 65%, 70%, 75%, 80%, 85%, 90%, 95%, 96%, 97%, 98%, 99% or 100% sequence identity with SEQ ID NO: 44.

In preferred embodiments, the transmembrane protein that can be bound by the second binding domain of the heterobifunctional molecule is VEGFR2. Preferably, the VEGFR2 protein has at least about 60%, 65%, 70%, 75%, 80%, 85%, 90%, 95%, 96%, 97%, 98%, 99% or 100% sequence identity with SEQ ID NO: 45. Preferably, the VEGFR2 protein is encoded by a sequence having at least about 60%, 65%, 70%, 75%, 80%, 85%, 90%, 95%, 96%, 97%, 98%, 99% 01100% sequence identity with SEQ ID NO: 46.

In preferred embodiments, the transmembrane protein that can be bound by the second binding domain of the heterobifunctional molecule is KIT. Preferably, the KIT protein has at least about 60%, 65%, 70%, 75%, 80%, 85%, 90%, 95%, 96%, 97%, 98%, 99% or 100% sequence identity with SEQ ID NO: 47. Preferably, the KIT protein is encoded by a sequence having at least about 60%, 65%, 70%, 75%, 80%, 85%, 90%, 95%, 96%, 97%, 98%, 99% or 100% sequence identity with SEQ ID NO: 48.

In preferred embodiments, the transmembrane protein that can be bound by the second binding domain of the heterobifunctional molecule is FLT3. Preferably, the FLT3 protein has at least about 60%, 65%, 70%, 75%, 80%, 85%, 90%, 95%, 96%, 97%, 98%, 99% or 100% sequence identity with SEQ ID NO: 49. Preferably, the FLT3 protein is encoded by a sequence having at least about 60%, 65%, 70%, 75%, 80%, 85%, 90%, 95%, 96%, 97%, 98%, 99% or 100% sequence identity with SEQ ID NO: 50.

In preferred embodiments, the transmembrane protein that can be bound by the second binding domain of the heterobifunctional molecule is PDGFRA. Preferably, the PDGFRA protein has at least about 60%, 65%, 70%, 75%, 80%, 85%, 90%, 95%, 96%, 97%, 98%, 99% or 100% sequence identity with SEQ ID NO: 53. Preferably, the PDGFRA protein is encoded by a sequence having at least about 60%, 65%, 70%, 75%, 80%, 85%, 90%, 95%, 96%, 97%, 98%, 99% or 100% sequence identity with SEQ ID NO: 54.

In preferred embodiments, the transmembrane protein that can be bound by the second binding domain of the heterobifunctional molecule is PDGFRB. Preferably, the PDGFRB protein has at least about 60%, 65%, 70%, 75%, 80%, 85%, 90%, 95%, 96%, 97%, 98%, 99% or 100% sequence identity with SEQ ID NO: 55. Preferably, the PDGFRB protein is encoded by a sequence having at least about 60%, 65%, 70%, 75%, 80%, 85%, 90%, 95%, 96%, 97%, 98%, 99% or 100% sequence identity with SEQ ID NO: 56.

In preferred embodiments, the transmembrane protein that can be bound by the second binding domain of the heterobifunctional molecule is selected from the group consisting of FZD1, FZD2, FZD3, FZD4, FZD5, FZD6, FZD7, FZD8, FZD9 and FZD10.

In preferred embodiments, the transmembrane protein that can be bound by the second binding domain of the heterobifunctional molecule is FZD1. Preferably, the FZD1 protein has at least about 60%, 65%, 70%, 75%, 80%, 85%, 90%, 95%, 96%, 97%, 98%, 99% or 100% sequence identity with SEQ ID NO: 57. Preferably, the FZD1 protein is encoded by a sequence having at least about 60%, 65%, 70%, 75%, 80%, 85%, 90%, 95%, 96%, 97%, 98%, 99% or 100% sequence identity with SEQ ID NO: 58.

In preferred embodiments, the transmembrane protein that can be bound by the second binding domain of the heterobifunctional molecule is FZD2. Preferably, the FZD2 protein has at least about 60%, 65%, 70%, 75%, 80%, 85%, 90%, 95%, 96%, 97%, 98%, 99% or 100% sequence identity with SEQ ID NO: 59. Preferably, the FZD2 protein is encoded by a sequence having at least about 60%, 65%, 70%, 75%, 80%, 85%, 90%, 95%, 96%, 97%, 98%, 99% or 100% sequence identity with SEQ ID NO: 60.

In preferred embodiments, the transmembrane protein that can be bound by the second binding domain of the heterobifunctional molecule is FZD3. Preferably, the FZD3 protein has at least about 60%, 65%, 70%, 75%, 80%, 85%, 90%, 95%, 96%, 97%, 98%, 99% or 100% sequence identity with SEQ ID NO: 61. Preferably, the FZD3 protein is encoded by a sequence having at least about 60%, 65%, 70%, 75%, 80%, 85%, 90%, 95%, 96%, 97%, 98%, 99% or 100% sequence identity with SEQ ID NO: 62.

In preferred embodiments, the transmembrane protein that can be bound by the second binding domain of the heterobifunctional molecule is FZD4. Preferably, the FZD4 protein has at least about 60%, 65%, 70%, 75%, 80%, 85%, 90%, 95%, 96%, 97%, 98%, 99% or 100% sequence identity with SEQ ID NO: 63. Preferably, the FZD4 protein is encoded by a sequence having at least about 60%, 65%, 70%, 75%, 80%, 85%, 90%, 95%, 96%, 97%, 98%, 99% or 100% sequence identity with SEQ ID NO: 64.

In preferred embodiments, the transmembrane protein that can be bound by the second binding domain of the heterobifunctional molecule is FZD5. Preferably, the FZD5 protein has at least about 60%, 65%, 70%, 75%, 80%, 85%, 90%, 95%, 96%, 97%, 98%, 99% or 100% sequence identity with SEQ ID NO: 65. Preferably, the FZD5 protein is encoded by a sequence having at least about 60%, 65%, 70%, 75%, 80%, 85%, 90%, 95%, 96%, 97%, 98%, 99% or 100% sequence identity with SEQ ID NO: 66.

In preferred embodiments, the transmembrane protein that can be bound by the second binding domain of the heterobifunctional molecule is FZD6. Preferably, the FZD6 protein has at least about 60%, 65%, 70%, 75%, 80%, 85%, 90%, 95%, 96%, 97%, 98%, 99% or 100% sequence identity with SEQ ID NO: 67. Preferably, the FZD6 protein is encoded by a sequence having at least about 60%, 65%, 70%, 75%, 80%, 85%, 90%, 95%, 96%, 97%, 98%, 99% or 100% sequence identity with SEQ ID NO: 68.

In preferred embodiments, the transmembrane protein that can be bound by the second binding domain of the heterobifunctional molecule is FZD7. Preferably, the FZD7 protein has at least about 60%, 65%, 70%, 75%, 80%, 85%, 90%, 95%, 96%, 97%, 98%, 99% or 100% sequence identity with SEQ ID NO: 69. Preferably, the FZD7 protein is encoded by a sequence having at least about 60%, 65%, 70%, 75%, 80%, 85%, 90%, 95%, 96%, 97%, 98%, 99% or 100% sequence identity with SEQ ID NO: 70.

In preferred embodiments, the transmembrane protein that can be bound by the second binding domain of the heterobifunctional molecule is FZD8. Preferably, the FZD8 protein has at least about 60%, 65%, 70%, 75%, 80%, 85%, 90%, 95%, 96%, 97%, 98%, 99% or 100% sequence identity with SEQ ID NO: 71. Preferably, the FZD8 protein is encoded by a sequence having at least about 60%, 65%, 70%, 75%, 80%, 85%, 90%, 95%, 96%, 97%, 98%, 99% or 100% sequence identity with SEQ ID NO: 72.

In preferred embodiments, the transmembrane protein that can be bound by the second binding domain of the heterobifunctional molecule is FZD9. Preferably, the FZD9 protein has at least about 60%, 65%, 70%, 75%, 80%, 85%, 90%, 95%, 96%, 97%, 98%, 99% or 100% sequence identity with SEQ ID NO: 73. Preferably, the FZD9 protein is encoded by a sequence having at least about 60%, 65%, 70%, 75%, 80%, 85%, 90%, 95%, 96%, 97%, 98%, 99% or 100% sequence identity with SEQ ID NO: 74.

In preferred embodiments, the transmembrane protein that can be bound by the second binding domain of the heterobifunctional molecule is FZD10. Preferably, the FZD10 protein has at least about 60%, 65%, 70%, 75%, 80%, 85%, 90%, 95%, 96%, 97%, 98%, 99% or 100% sequence identity with SEQ ID NO: 75. Preferably, the FZD10 protein is encoded by a sequence having at least about 60%, 65%, 70%, 75%, 80%, 85%, 90%, 95%, 96%, 97%, 98%, 99% or 100% sequence identity with SEQ ID NO: 76.

In preferred embodiments, the transmembrane protein that can be bound by the second binding domain of the heterobifunctional molecule is LRP5 or LRP6.

In preferred embodiments, the transmembrane protein that can be bound by the second binding domain of the heterobifunctional molecule is LRP5. Preferably, the LRP5 protein has at least about 60%, 65%, 70%, 75%, 80%, 85%, 90%, 95%, 96%, 97%, 98%, 99% or 100% sequence identity with SEQ ID NO: 77. Preferably, the LRP5 protein is encoded by a sequence having at least about 60%, 65%, 70%, 75%, 80%, 85%, 90%, 95%, 96%, 97%, 98%, 99% or 100% sequence identity with SEQ ID NO: 78.

In preferred embodiments, the transmembrane protein that can be bound by the second binding domain of the heterobifunctional molecule is LRP6. Preferably, the LRP6 protein has at least about 60%, 65%, 70%, 75%, 80%, 85%, 90%, 95%, 96%, 97%, 98%, 99% or 100% sequence identity with SEQ ID NO: 79. Preferably, the LRP6 protein is encoded by a sequence having at least about 60%, 65%, 70%, 75%, 80%, 85%, 90%, 95%, 96%, 97%, 98%, 99% or 100% sequence identity with SEQ ID NO: 80.

In preferred embodiments, the transmembrane protein that can be bound by the second binding domain of the heterobifunctional molecule is Growth Hormone Receptor (GHR). Preferably, the GHR protein has at least about 60%, 65%, 70%, 75%, 80%, 85%, 90%, 95%, 96%, 97%, 98%, 99% or 100% sequence identity with SEQ ID NO: 84. Preferably, the GHR protein is encoded by a sequence having at least about 60%, 65%, 70%, 75%, 80%, 85%, 90%, 95%, 96%, 97%, 98%, 99% or 100% sequence identity with SEQ ID NO: 85.

In preferred embodiments, the transmembrane protein functions as an immune checkpoint inhibitor. Preferably, the transmembrane protein that can be bound by the second binding domain of the heterobifunctional molecule is selected from the group consisting of PD-1, PD-L1, CTLA4, CMTM6, CMTM4 and WLS.

In preferred embodiments, the transmembrane protein that can be bound by the second binding domain of the heterobifunctional molecule is PD-1. Preferably, the PD-1 protein has at least about 60%, 65%, 70%, 75%, 80%, 85%, 90%, 95%, 96%, 97%, 98%, 99% or 100% sequence identity with SEQ ID NO: 86. Preferably, the PD-1 protein is encoded by a sequence having at least about 60%, 65%, 70%, 75%, 80%, 85%, 90%, 95%, 96%, 97%, 98%, 99% or 100% sequence identity with SEQ ID NO: 87.

In preferred embodiments, the transmembrane protein that can be bound by the second binding domain of the heterobifunctional molecule is PD-L1. Preferably, the PD-L1 protein has at least about 60%, 65%, 70%, 75%, 80%, 85%, 90%, 95%, 96%, 97%, 98%, 99% or 100% sequence identity with SEQ ID NO: 88. Preferably, the PD1L1 protein is encoded by a sequence having at least about 60%, 65%, 70%, 75%, 80%, 85%, 90%, 95%, 96%, 97%, 98%, 99% or 100% sequence identity with SEQ ID NO: 89.

In preferred embodiments, the transmembrane protein that can be bound by the second binding domain of the heterobifunctional molecule is CTLA4. Preferably, the CTLA4 protein has at least about 60%, 65%, 70%, 75%, 80%, 85%, 90%, 95%, 96%, 97%, 98%, 99% or 100% sequence identity with SEQ ID NO: 90. Preferably, the CTLA4 protein is encoded by a sequence having at least about 60%, 65%, 70%, 75%, 80%, 85%, 90%, 95%, 96%, 97%, 98%, 99% or 100% sequence identity with SEQ ID NO: 91.

In preferred embodiments, the transmembrane protein that can be bound by the second binding domain of the heterobifunctional molecule is CMTM6. Preferably, the CMTM6 protein has at least about 60%, 65%, 70%, 75%, 80%, 85%, 90%, 95%, 96%, 97%, 98%, 99% or 100% sequence identity with SEQ ID NO: 92. Preferably, the CMTM6 protein is encoded by a sequence having at least about 60%, 65%, 70%, 75%, 80%, 85%, 90%, 95%, 96%, 97%, 98%, 99% or 100% sequence identity with SEQ ID NO: 93.

In preferred embodiments, the transmembrane protein that can be bound by the second binding domain of the heterobifunctional molecule is CMTM4. Preferably, the CMTM4 protein has at least about 60%, 65%, 70%, 75%, 80%, 85%, 90%, 95%, 96%, 97%, 98%, 99% or 100% sequence identity with SEQ ID NO: 94. Preferably, the CMTM4 protein is encoded by a sequence having at least about 60%, 65%, 70%, 75%, 80%, 85%, 90%, 95%, 96%, 97%, 98%, 99% or 100% sequence identity with SEQ ID NO: 95.

In preferred embodiments, the transmembrane protein that can be bound by the second binding domain of the heterobifunctional molecule is WLS/GPR177. Preferably, the WLS protein has at least about 60%, 65%, 70%, 75%, 80%, 85%, 90%, 95%, 96%, 97%, 98%, 99% or 100% sequence identity with SEQ ID NO: 100. Preferably, the WLS protein is encoded by a sequence having at least about 60%, 65%, 70%, 75%, 80%, 85%, 90%, 95%, 96%, 97%, 98%, 99% or 100% sequence identity with SEQ ID NO: 101.

In preferred embodiments, the transmembrane protein that can be bound by the second binding domain of the heterobifunctional molecule is SLC7A5. Preferably, the SLC7A5 protein has at least about 60%, 65%, 70%, 75%, 80%, 85%, 90%, 95%, 96%, 97%, 98%, 99% or 100% sequence identity with SEQ ID NO: 102. Preferably, the SLC7A5 protein is encoded by a sequence having at least about 60%, 65%, 70%, 75%, 80%, 85%, 90%, 95%, 96%, 97%, 98%, 99% or 100% sequence identity with SEQ ID NO: 103.

In preferred embodiments, the transmembrane protein that can be bound by the second binding domain of the heterobifunctional molecule is SLC16A7 (MCT2). Preferably, the SLC16A7 protein has at least about 60%, 65%, 70%, 75%, 80%, 85%, 90%, 95%, 96%, 97%, 98%, 99% or 100% sequence identity with SEQ ID NO: 104. Preferably, the SLC16A7 protein is encoded by a sequence having at least about 60%, 65%, 70%, 75%, 80%, 85%, 90%, 95%, 96%, 97%, 98%, 99% or 100% sequence identity with SEQ ID NO: 105.

Preferably, the second binding domain of the heterobifunctional molecule binds an extracellular portion of the membrane-bound protein. Hence preferably, the heterobifunctional molecule does not have to cross the cell membrane to bind the membrane-bound protein.

Preferably, the first and second binding domain of the heterobifunctional molecule binds an extracellular portion of respectively the transmembrane E3 ubiquitin ligase and the membrane-bound protein. Preferably, the heterobifunctional molecule binds extracellularly.

### First Binding Domain

The heterobifunctional molecule of the invention comprises at least a first binding domain and a second binding domain. The first binding domain is capable of specific binding to a transmembrane E3 ubiquitin ligase. Preferably, the first binding domain is capable of specific binding to a transmembrane E3 ubiquitin ligase as specified above in the section “Protein bound by the first binding domain”. The first binding domain of the heterobifunctional molecule may selectively bind to a native epitope of the transmembrane E3 ubiquitin ligase, preferably when the heterobifunctional molecule is for use in a medicament. Alternatively, the first binding domain of the heterobifunctional molecule may selectively bind to a non-native epitope engineered into the transmembrane E3 ubiquitin ligase, preferably when the heterobifunctional molecule is used in a method of the invention, preferably a selection method of the invention.

The first binding domain of the heterobifunctional molecule can be any domain capable of specific binding to the transmembrane E3 ubiquitin ligase. Preferably, the first binding domain of the heterobifunctional molecule binds to an extracellular portion of the transmembrane E3 ubiquitin ligase.

The skilled person understands how to generate a first binding domain of a heterobifunctional molecule of the invention, e.g. by means of screening compound libraries, immunization studies and/or hybridoma technology to generate antibodies or functional fragments thereof. A preferred functional antibody fragment is a nanobody. Details of these techniques are e.g. described in (Antibodies: A Laboratory Manual, Harlow et al., Cold Spring Harbor Publications, p. 726, 1988), or are described by (Campbell, A. M. “Monoclonal Antibody Technology Techniques in Biochemistry and Molecular Biology,” Elsevier Science Publishers, Amsterdam, The Netherlands, 1984) or by (St. Groth et al., J. Immunol. Methods 35:1-21, 1980). Details for the generation of VHH/nanobodies against native epitopes is e.g. described in Pardon et al, Nature Protocols 2014, which is incorporated herein by reference.

In preferred embodiments, the molecule that can bind to a transmembrane E3 ubiquitin ligase is an antibody. Hence preferably, the antibody may function as a first binding domain in the heterobifunctional molecule of the invention.

Preferably, the antibody is an antibody fragment. Preferably, the antibody fragment is a nanobody. Hence in preferred embodiments the molecule that can bind to a transmembrane E3 ubiquitin ligase is a nanobody. Hence preferably, a nanobody may function as a first binding domain in the heterobifunctional molecule of the invention.

In preferred embodiments, the first binding domain is a small organic molecule.

In preferred embodiments, the first binding domain is an aptamer.

In preferred embodiments, the first binding domain is a proteinaceous molecule. The proteinaceous molecule may be cyclised, hence preferably the proteinaceous molecule is a cyclic peptide. The peptide may be cyclised by direct covalent linkage between two amino acid residues or by using a cross-linking moiety. Such cross-linking moieties are well-known in the art, such as, but not limited to the cross linking moieties described in WO2012/057624, which is incorporated herein by reference. The proteinaceous molecule can be proteinaceous molecule previously known in the art.

The invention thus extends to molecules known in the art for specific binding to a transmembrane E3 ubiquitin ligase, which molecules can function as a first binding domain of the heterobifunctional molecule of the invention. Such known molecules include, but are not limited to, at least one of a known antibody, proteinaceous molecule, aptamer or known small organic molecule. Preferably, the antibody, proteinaceous molecule, aptamer or small organic molecule is known in the art for binding to an extracellular portion of the transmembrane E3 ubiquitin ligase.

Antibodies binding to a transmembrane E3 ubiquitin ligase are known in the art and the skilled person would have no difficulties retrieving such antibodies. Any known antibody capable of specific binding to a transmembrane E3 ubiquitin ligase, preferably capable of specific binding to the extracellular portion of a transmembrane E3 ubiquitin ligase, will be suitable for use as a first binding domain in the heterobifunctional molecule of the current invention.

A preferred known molecule that can bind to a transmembrane E3 ubiquitin ligase is a nanobody. Hence preferably, a nanobody may function as a first binding domain in a heterobifunctional molecule of the invention.

In preferred embodiments, the first binding domain is a natural ligand of the transmembrane E3 ubiquitin ligase, or a functional fragment thereof, i.e. a fragment of the natural ligand that remains capable of binding the transmembrane E3 ubiquitin ligase.

As a non-limiting example, natural ligands for RNF43 and ZNRF3 are Rspondin (RSPO)-1, -2, -3 and -4. Hence in an embodiment, the heterobifunctional molecule comprises a first binding domain that is capable of binding to RNF43, wherein the first binding domain is selected from the group consisting of Rspondin 1, Rspondin 2, Rspondin 3 and Rspondin 4, or a functional fragment thereof. Alternatively, the heterobifunctional molecule comprises a first binding domain that is capable of binding to ZNRF3, wherein the first binding domain is selected from the group consisting of Rspondin 1, Rspondin 2, Rspondin 3 and Rspondin 4, or a functional fragment thereof.

### Second Binding Domain

The heterobifunctional molecule of the invention comprises at least a first binding domain and a second binding domain. The second domain is capable of specific binding to a membrane-bound protein, preferably a transmembrane protein. Preferably, the second binding domain is capable of specific binding to a membrane-bound protein as specified above in the section “Protein bound by the second binding domain”. The second binding domain of the heterobifunctional molecule may selectively bind to a native epitope of the membrane-bound protein, preferably when the heterobifunctional molecule is for use in a medicament. Alternatively, the second binding domain of the heterobifunctional molecule may selectively bind to a non-native epitope engineered into the membrane-bound protein, preferably when the heterobifunctional molecule is used in a method of the invention, preferably a selection method of the invention.

The second binding domain of the heterobifunctional molecule can be any domain capable of specific binding to the membrane-bound protein, preferably to a transmembrane membrane protein. Preferably, the second binding domain of the heterobifunctional molecule binds to an extracellular portion of the membrane-bound protein.

The second binding domain can be an antibody, a peptide, an aptamer or a small organic molecule.

The skilled person understands how to generate a second binding domain of a heterobifunctional molecule of the invention e.g. by means of screening compound libraries, immunization studies and/or hybridoma technology to generate antibodies or functional fragments thereof. A preferred functional antibody fragment is a nanobody. Details of these techniques are e.g. described in (Antibodies: A Laboratory Manual, Harlow et al., Cold Spring Harbor Publications, p. 726, 1988), or are described by (Campbell, A. M. “Monoclonal Antibody Technology Techniques in Biochemistry and Molecular Biology,” Elsevier Science Publishers, Amsterdam, The Netherlands, 1984) or by (St. Groth et al., J. Immunol. Methods 35:1-21, 1980).

In preferred embodiments, the molecule that can bind to a membrane-bound protein is an antibody. Hence preferably, the antibody may function as a second binding domain in the heterobifunctional molecule of the invention.

Preferably, the antibody is an antibody fragment. Preferably, the antibody fragment is a nanobody. Hence in preferred embodiments the molecule that can bind to a membrane-bound protein is a nanobody. Hence preferably, a nanobody may function as a second binding domain in the heterobifunctional molecule of the invention.

In preferred embodiments, the first binding domain is a small organic molecule.

In preferred embodiments, the first binding domain is an aptamer.

In preferred embodiments, the second binding domain is a proteinaceous molecule. The proteinaceous molecule may be cyclised, hence preferably the proteinaceous molecule is a cyclic peptide. The peptide may be cyclised by direct covalent linkage between two amino acid residues or by using a cross-linking moiety. Such cross-linking moieties are well-known in the art, such as, but not limited to the cross linking moieties described in WO2012/057624, which is incorporated herein by reference. The proteinaceous molecule can be proteinaceous molecule previously known in the art.

The invention thus extends to molecules known in the art for specific binding to a membrane-bound protein, preferably a membrane-bound protein as defined herein above. Such molecules can function as a second binding domain of the heterobifunctional molecule of the invention.

Such known molecules include, but are not limited to, at least one of a known antibody, proteinaceous molecule, aptamer or known small organic molecule. Preferably, the antibody, proteinaceous molecule, aptamer or small organic molecule is known in the art for binding to an extracellular portion of a membrane-bound protein as defined herein.

Antibodies binding to membrane-bound proteins, preferably transmembrane proteins as defined herein above, are known in the art and the skilled person would have no difficulties retrieving such antibodies. Any known antibody capable of specific binding to a membrane-bound protein as defined herein, preferably capable of specific binding to the extracellular portion of a membrane-bound protein as defined herein, will be suitable for use as a second binding domain in the heterobifunctional molecule of the current invention.

A preferred known molecule that can bind to a membrane-bound protein is a nanobody. Hence preferably, a nanobody may function as a second binding domain in a heterobifunctional molecule of the invention.

In preferred embodiments, the second binding domain is a natural ligand of the membrane-bound protein, preferably a transmembrane protein as defined herein. Preferable, the natural ligand is an antagonist of the transmembrane protein.

### Heterobifunctional Molecule

The first and second binding domain are capable to specifically bind respectively a transmembrane protein or a membrane-bound protein, i.e. the target proteins.

Specific binding is herein understood as that the extent of binding of the domains of a “non-target” protein will be less than about 10%, 9%, 8%, 7%, 6%, 5%, 4%, 3%, 2% or 1% of the binding of the domains to its particular target protein as determined by fluorescence activated cell sorting (FACS) analysis, ELISA or radioimmunoprecipitation (RIA). With regard to the binding of the domains to a target protein, the term “specific binding” or “specifically binds to” or is “specific for” a particular polypeptide or an epitope on a particular polypeptide target means binding that is measurably different from a non-specific interaction. Specific binding can be measured, for example, by determining binding of a target protein as compared to binding of a control protein, which generally is a protein of similar structure that does not have binding activity. For example, specific binding can be determined by competition with a control protein that is similar to the target, for example, an excess of non-labelled target. In this case, specific binding is indicated if the binding of the labelled target to a probe is competitively inhibited by excess unlabelled target.

The term “specific binding” or “specifically binds to” or is “specific for” a particular polypeptide or an epitope on a particular polypeptide target as used herein can be exhibited, for example, by a binding domain having a K_{d} for the target (which may be determined as described above) of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a binding domain binds to a particular polypeptide or epitope on a particular polypeptide without substantially binding to any other polypeptide or polypeptide epitope.

The result of simultaneous binding of the heterobifunctional molecule to the transmembrane E3 ubiquitin ligase and the membrane-bound protein results in ubiquitination and degradation of the transmembrane protein. Preferably, the membrane-bound protein is a transmembrane protein. Hence preferably, the result of simultaneous binding of the heterobifunctional molecule to the transmembrane E3 ubiquitin ligase and the transmembrane protein results in ubiquitination and degradation of the transmembrane protein. Preferably, the degradation is at least one of proteasomal degradation and lysosomal degradation. Preferably, the degradation is lysosomal degradation.

A heterobifunctional molecule as defined herein thus may knock-down or knock-out the presence of a membrane-bound protein on the cell membrane by bringing the transmembrane E3 ubiquitin ligase in close proximity of the target, i.e. the membrane-bound protein. Put differently, the steady state level of the membrane-bound protein will be reduced.

The steady state level may be defined herein as the abundance of the protein per cell. As compared to a reference cell, the steady state level of the membrane-bound protein may be reduced at least about 1%, 5%, 10%, 15%, 20%, 25%, 30%, 35%, 40%, 45%, 50%, 55%, 60%, 65%, 70%, 75%, 80%, 85%, 90%, 95% or may be reduced about 100%, i.e. binding of the heterobifunctional molecule leads to a complete absence of the membrane-bound protein.

In preferred embodiments, the heterobifunctional molecule is a bispecific antibody. Bispecific antibodies are e.g. described in Wu X and Demarest S J, Methods. (2019)154:3-9. Hence preferably, the first binding domain is an antibody capable of specific binding to a transmembrane E3 ubiquitin ligase, preferably a transmembrane E3 ubiquitin ligase as specified in the section “Transmembrane E3 ubiquitin ligase bound by a first binding domain” above. Preferably the second binding domain is also an antibody, wherein the antibody is capable of specific binding to a membrane-bound protein, preferably a transmembrane protein, preferably a transmembrane protein as specified in the section “Protein bound by the second binding domain” above. The two antibodies (i.e. the first and second binding domains) can be coupled directly together, or there can be a linker present between the two antibodies, preferably a linker as specified herein.

In preferred embodiments, the heterobifunctional molecule is a bi-specific nanobody. Bi-specific nanobodies are e.g. disclosed in WO 2015/044386 and Conrath et al (Camel Single-domain Antibodies as Modular Building Units in Bispecific and Bivalent Antibody Constructs, JBC, 2001). Preferably, the first binding domain is a nanobody capable of specific binding to a transmembrane E3 ubiquitin ligase, preferably a transmembrane E3 ubiquitin ligase as specified in the section “Transmembrane E3 ubiquitin ligase bound by a first binding domain” above. Preferably the second binding domain is also a nanobody, wherein the nanobody is capable of specific binding to a membrane-bound protein, preferably a transmembrane protein, preferably a transmembrane protein as specified in the section “Protein bound by the second binding domain” above. The two nanobodies (i.e. the first and second binding domains) can be coupled directly together, or there can be a linker present between the two nanobodies, preferably a linker as specified herein.

In preferred embodiments, the heterobifunctional molecule is a bicyclic peptide. Preferably, the first binding domain is a cyclic peptide capable of specific binding to a transmembrane E3 ubiquitin ligase, preferably a transmembrane E3 ubiquitin ligase as specified in the section “Transmembrane E3 ubiquitin ligase bound by a first binding domain” above. Preferably the second binding domain is also a cyclic peptide, wherein the cyclic peptide is capable of specific binding to a membrane-bound protein, preferably a transmembrane protein, preferably a transmembrane protein as specified in the section “Protein bound by the second binding domain” above. The two cyclic peptides (i.e. the first and second binding domains) can be coupled directly together, e.g. by using the same cross-linking moiety, or there can be a linker present between the two cyclic peptides, preferably a linker as specified herein.

The heterobifunctional molecule may optionally comprise a moiety to increase the stability of the molecule. Such moiety includes, but is not limited to, a binding domain for the specific binding of albumin.

The heterobifunctional molecule may optionally comprise a tag, preferably a peptide- or protein-tag as defined herein, for purification or detection of the heterobifunctional molecule. A preferred purification tag is a His-tag or an Avi-tag. A preferred detection tag is a V5-tag. Such heterobifunctional molecule would be particularly useful for use in the (screening) method of the invention.

The heterobifunctional molecule of the invention may comprise a linker between the first binding domain and the second binding domain. The linker may be any suitable linker known in the art. Preferably, the linker is a Gly-Ser sequence. The skilled person knows how to select the linker, dependent on the first binding domain and the second binding domain. The linker may be e.g. a very flexible linker in the form (GGGGS)n, (GGS)n, and (G)n to more rigid linkers of the form (EAAAK)n, (SPKKKRKVEAS)n (SEQ ID NO: 81), or (SGSETPGTSESATPES)n (SEQ ID NO: 82), or (KSGSETPGTSESATPES)n (SEQ ID NO: 83), or any variant thereof, wherein n preferably is between 1 and 15, preferably between 1 and 7, i.e. 1, 2, 3, 4, 5, 6, 7, 8, 9, 10, 11, 12, 13, 14 or 15.

The linker preferably has a length between 2 and 250 amino acids, between 2 and 30 amino acids, or between 3 and 23 amino acids, or between 3 and 18 amino acids.

The linker may be a cleavable linker, for example by introducing a 3C protease cleavage site in the linker. Such linker would be particularly useful for use in the (screening) method of the invention as it provides for a control, e.g. by cleaving the linker the forced dimerization should be abolished.

### Therapeutic Uses

The heterobifunctional molecule as defined herein can be used for decreasing the level of any selected membrane-bound protein by simultaneous binding of a transmembrane ubiquitin E3 ligase and the selected membrane-bound protein. The combination of the transmembrane ubiquitin ligase and the selected membrane-bound protein is preferably an effective combination, preferably selected using the selection method of the invention.

In an aspect, the heterobifunctional molecule as defined herein is for use as a medicament. The heterobifunctional molecule for use as a medicament preferably binds to a native epitope present on the transmembrane ubiquitin E3 ligase and to a native epitope present on the selected membrane-bound protein. The medical use herein described is formulated as a heterobifunctional molecule as defined herein for use as a medicament for treatment of the stated disease(s) by administration of an effective amount of the heterobifunctional molecule, but could equally be formulated as a method of treatment of the stated disease(s) using a heterobifunctional molecule as defined herein comprising a step of administering to a subject an effective amount of the heterobifunctional molecule, a heterobifunctional molecule as defined herein for use in the preparation of a medicament to treat the stated disease(s) wherein the heterobifunctional molecule is to be administered in an effective amount and use of a heterobifunctional molecule as defined herein for the treatment of the stated disease(s) by administering an effective amount. Such medical uses are all envisaged by the present invention.

The skilled person understands that an increased activity of any membrane-bound protein involved in the onset, severity, or duration of a disease can be a suitable target for a heterobifunctional molecule as defined herein. Hence, the heterobifunctional molecule is not limited to any specific membrane-bound protein or any specific disease. Preferably, the disease is characterized in that the activity of a membrane-bound protein, preferably a receptor, is increased, wherein the increased activity of the membrane-bound receptor preferably influences or dictates the onset, severity or duration of a disease.

As a non-limiting example, the heterobifunctional molecule may be used for the treatment of at least one of cancer, dementia, heart disease, neural disorder, rare disease and an infectious disease.

Increased activity of membrane-bound receptors are well-known in the art to play a significant role in e.g. the onset, severity or duration of cancer. Hence in an embodiment, the heterobifunctional molecule is used for the treatment, prophylaxis, reduction, or suppression of symptoms associated with cancer.

Preferably, the cancer is a cancer as defined herein above in the section “Protein bound by the second binding domain”.

Preferably, the cancer is a solid cancer or a hematologic cancer. Alternatively or in addition, the receptor may be involved in cancer angiogenesis.

Preferably, the solid cancer is a solid cancer as defined herein above in the section “Protein bound by the second binding domain”.

Preferably, the hematologic cancer is a hematologic cancer as defined herein above in the section “Protein bound by the second binding domain”.

In an aspect, the invention pertains to a composition comprising a heterobifunctional molecule as defined herein. The heterobifunctional molecule preferably binds to an effective combination of a transmembrane ubiquitin E3 ligase and a membrane-bound protein, preferably selected using the selection method of the invention. The composition may be suitable for use in cell culture, preferably animal cell culture, more preferably mammalian cell culture. In addition or alternatively, the composition preferably is a pharmaceutical composition or a cosmetic composition.

The composition may comprise one type of heterobifunctional molecule or may comprise at least two different types of heterobifunctional molecules, e.g. to knock-down or knock-out the presence of two or more different membrane-bound proteins. The composition may comprise at least 1, 2, 3, 4, 5, 6, 7, 8, 9, 10 or more different types of heterobifunctional molecules.

The compositions comprising a heterobifunctional molecule as described above, can be prepared as a medicinal or cosmetic preparation or in various other media, such as foods for humans or animals, including medical foods and dietary supplements.

A “medical food” is a product that is intended for the specific dietary management of a disease or condition for which distinctive nutritional requirements exist. By way of example, but not limitation, medical foods may include vitamin and mineral formulations fed through a feeding tube (referred to as enteral administration).

A “dietary supplement” shall mean a product that is intended to supplement the human diet and is typically provided in the form of a pill, capsule, and tablet or like formulation. By way of example, but not limitation, a dietary supplement may include one or more of the following ingredients: vitamins, minerals, herbs, botanicals; amino acids, dietary substances intended to supplement the diet by increasing total dietary intake, and concentrates, metabolites, constituents, extracts or combinations of any of the foregoing. Dietary supplements may also be incorporated into food, including, but not limited to, food bars, beverages, powders, cereals, cooked foods, food additives and candies.

The subject composition thus may be compounded with other physiologically acceptable materials which can be ingested including, but not limited to, foods. In addition or alternatively, the compositions for use as described herein may be administered orally in combination with (the separate) administration of food.

The compositions may be administered alone or in combination with other pharmaceutical or cosmetic agents and can be combined with a physiologically acceptable carrier thereof. In particular, the heterobifunctional molecule described herein can be formulated as pharmaceutical or cosmetic compositions by formulation with additives such as pharmaceutically or physiologically acceptable excipients carriers, and vehicles.

Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003), 21^{st} edition (2005) and 22^{nd} edition (2012), incorporated herein by reference.

Pharmaceutical or cosmetic compositions containing the heterobifunctional molecule for use according to the invention may be in any form suitable for the intended method of administration, including, for example, a solution, a suspension, or an emulsion. In a preferred embodiment, the heterobifunctional molecule is administered in a solid form or in a liquid form.

Solid dosage forms for oral administration may include capsules, tablets, pills, powders, and granules. In such solid dosage forms, the heterobifunctional molecule may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water or saline. Such compositions may also comprise adjuvants, such as wetting agents, emulsifying and suspending agents, cyclodextrins, and sweetening, flavoring, and perfuming agents.

Liquid carriers are typically used in preparing solutions, suspensions, and emulsions. In a preferred embodiment, liquid carriers/liquid dosage forms contemplated for use in the practice of the present invention include, for example, water, saline, pharmaceutically acceptable organic solvent(s), pharmaceutically acceptable oils or fats, and the like, as well as mixtures of two or more thereof. In a preferred embodiment, the heterobifunctional molecule of the invention as defined herein is admixed with an aqueous solution prior to administration. The aqueous solution should be suitable for administration and such aqueous solutions are well known in the art. It is further known in the art that the suitability of an aqueous solution for administration may be dependent on the route of administration.

In a preferred embodiment, the aqueous solution is an isotonic aqueous solution. The isotonic aqueous solution preferably is almost (or completely) isotonic to blood plasma. In an even more preferred embodiment, the isotonic aqueous solution is saline.

The liquid carrier may contain other suitable pharmaceutically acceptable additives such as solubilizers, emulsifiers, nutrients, buffers, preservatives, suspending agents, thickening agents, viscosity regulators, stabilizers, flavorants and the like. Preferred flavorants are sweeteners, such as monosaccharides and/or disaccharides. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like.

For parenteral administration, the carrier can also be an oily ester such as ethyl oleate, isopropyl myristate, and the like. Compositions for use in the present invention may also be in the form of microparticles, microcapsules, liposomal encapsulates, and the like, as well as combinations of any two or more thereof.

Time-release, sustained release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The heterobifunctional molecules of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a heterobifunctional molecule as defined herein, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N. Y., p. 33 et seq (1976).

A pharmaceutical or cosmetic composition can comprise a unit dose formulation, where the unit dose is a dose sufficient to have a therapeutic or suppressive effect of a disorder or condition as defined herein, and/or an amount effective to reduce, or knock out, the expression of a membrane-bound protein. The unit dose may be sufficient as a single dose to have a therapeutic or suppressive effect of a disorder or condition as defined herein and/or an amount effective to reduce the expression of the target membrane-bound protein. Alternatively, the unit dose may be a dose administered periodically in a course of treatment or suppression of a disorder or condition as defined herein. During the course of the treatment, the concentration of the subject compositions may be monitored to insure that the desired level is maintained.

The heterobifunctional molecule or composition comprising a heterobifunctional molecule as defined herein may be administered enterally, orally, parenterally, sublingually, by inhalation (e. g. as mists or sprays), rectally, or topically, preferably in dosage unit formulations containing conventional nontoxic pharmaceutically or physiologically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e. g. via nasal mucosa), subdural, rectal, gastrointestinal, and the like, and directly to a specific or affected organ or tissue, e.g. a cancerous tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrasternal injection, or infusion techniques.

The heterobifunctional molecule can be mixed with pharmaceutically acceptable carriers, adjuvants, and vehicles appropriate for the desired route of administration. The heterobifunctional molecules of the invention may be administered by supplementation via gastric or percutaneous tubes.

In a preferred embodiment the invention pertains to a heterobifunctional molecule as defined herein above, for use in treating, preventing, or suppressing symptoms associated with a cancer by administration of an effective total daily dose.

The dosage form for oral administration can be a solid oral dosage form. The class of solid oral dosage forms consists primarily of tablets and capsules, although other forms are known in the art and can be equally suitable. When used as a solid oral dosage form, the heterobifunctional molecule as defined herein may e.g. be administered in the form of an immediate release tablet (or a capsule and the like) or a sustained release tablet (or a capsule and the like). Any suitable immediate release or sustained release solid dosage forms can be used in the context of the invention as will be evident for the skilled person.

The heterobifunctional molecule described for use as described herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The c heterobifunctional molecule can also be administered in liposome formulations. The compounds can also be administered as prodrugs, where the prodrug undergoes transformation in the treated subject to a form which is therapeutically effective. Additional methods of administration are known in the art.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the heterobifunctional molecule can be prepared by mixing the heterobifunctional molecule with a suitable nonirritating excipient such as cocoa butter and polyethylene glycols that are solid at room temperature but liquid at the rectal temperature and will therefore melt in the rectum and release the heterobifunctional molecule.

While the heterobifunctional molecules for use as described herein can be administered as the sole active pharmaceutical (or cosmetic) agent, they can also be used in combination with one or more other agents used in the treatment or suppression of a disease or a disorder. Representative agents useful in combination with the heterobifunctional molecule of the invention for the treating, preventing, or suppressing symptoms associated with a disease or disorder include, but are not limited to, Coenzyme Q, vitamin E, idebenone, MitoQ, EPI-743, vitamin K and analogues thereof, naphtoquinones and derivatives thereof, other vitamins, and antioxidant compounds.

When additional active agents are used in combination with the heterobifunctional molecule of the present invention, the additional active agents may generally be employed in therapeutic amounts as indicated in the Physicians' Desk Reference (PDR) 53rd Edition (1999), which is incorporated herein by reference, or such therapeutically useful amounts as would be known to one of ordinary skill in the art. The heterobifunctional molecule of the invention and the other therapeutically active agent or agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

### Manufacturing the Heterobifunctional Molecule

In an aspect, the invention pertains to a method for manufacturing a heterobifunctional molecule of the invention, wherein the method comprises the steps of:
Selecting an effective combination of a transmembrane E3 ubiquitin ligase and a membrane-bound protein, preferably using the method of the invention;
constructing a first binding domain capable of specific binding to the transmembrane E3 ubiquitin ligase;
constructing a second binding domain capable of specific binding to the membrane-bound protein; and
coupling the first binding domain to the second binding domain, wherein preferably the coupling is a direct coupling or through a linker, preferably a linker as defined herein.

It is understood herein that the step of constructing a first binding domain and a second binding domain can be performed using any conventional means in the art. As a non-limiting example, at least one of the first and second binding domain is a binding domain previously known in the art, e.g. an antibody known in the art for specific binding to a transmembrane E3 ubiquitin ligase or an antibody known in the art for specific binding to a membrane-bound protein.

Alternatively, at least one of the first and second binding domain is a de novo binding domain, for example but not limited to antibody or nanobody binding domains discovered by immunization studies.

The step of selecting a transmembrane E3 ubiquitin ligase and a membrane-bound protein may be accomplished by incorporating a first non-native epitope tag in the transmembrane E3 ubiquitin ligase and incorporating a second non-native epitope tag in the membrane-bound protein. Upon expression in the cell, the first and second epitope tags are preferably displayed at the respective extracellular portion, i.e. are displayed extracellularly. A heterobifunctional molecule having a first binding domain capable of binding the first epitope tag and a second binding domain capable of binding the second epitope tag may subsequently be used to assess the potency of targeting the transmembrane E3 ubiquitin ligase to the membrane-bound protein. The potency may be assessed by determining to which extent the transmembrane E3 ubiquitin ligase is capable of ubiquitination and internalisation of the membrane-bound protein after forced interaction between the E3 ubiquitin ligase and the membrane-bound protein using a heterobifunctional molecule. In addition or alternatively, the potency may be assessed by determining to which extent the cell surface levels and/or total protein levels of the membrane-bound protein are decreased after forced interaction between the E3 ubiquitin ligase and the membrane-bound protein using this selection system. The step of selecting a transmembrane E3 ubiquitin ligase and membrane-bound protein may also be considered a method for decreasing the surface level of a membrane-bound protein of a cell as detailed herein below.

Following the selection of an effective combination of a transmembrane E3 ubiquitin ligase and a membrane-bound protein, a heterobifunctional molecule may be constructed comprising a first binding domain capable of specific binding to the extracellular portion of a (native) transmembrane E3 ubiquitin ligase and a second binding domain capable of specific binding to the extracellular portion of a (native) membrane-bound protein.

### FIGURE LEGENDS

FIG. 1. Schematic representation of an exemplary embodiment of the invention. A heterobifunctional molecule of the invention simultaneously binds to a transmembrane E3 ubiquitin ligase and a transmembrane protein. As a consequence, the transmembrane protein will become ubiquitinated, internalized and degraded.

FIG. 2. Functional assessment of the A/C dimerizer. HEK293T cells were transfected with RNF43-FKBP and TβRII-Flag-FRB and treated with the A/C dimerizer or a similar volume of 100% ethanol overnight. Flag-M2 beads were used to immunoprecipitate the TβRII construct from the cell lysates. IP samples and whole cell lysates were separated by SDS-page, blotted and stained for Flag and RNF43 to detect binding between the two constructs.

FIG. 3. Forced dimerization of RNF43 and TβRII induces the relocalization of both proteins to perinuclear lysosomes. (a) Confocal images of HEK293T cells transfected with TβRII-Flag-FRB. (B) Confocal images of HEK293T cells transfected with RNF43-FKBP and TβRII-Flag-FRB. Cells were treated with A/C dimerizer or a similar volume of 100% ethanol overnight. TβRII and RNF43 were visualized by Flag and RNF43 staining, respectively. (C) Confocal images of HEK293T cells transfected with CD63-GFP, RNF43-FKBP and TβRII-Flag-FRB. Cells were treated with A/C dimerizer overnight and TβRII-Flag-FRB was visualized by Flag staining. Arrows indicate perinuclear lysosomes.

FIG. 4. TβRII is degraded upon forced dimerization of RNF43 and TβRII. HEK293T cells were transfected with RNF43-FKBP and TβRII-Flag-FRB and treated with the A/C dimerizer or a similar volume of 100% ethanol overnight. Cell lysates were separated by SDS-page, blotted and stained for Flag and RNF43 to visualize protein levels.

FIG. 5. VHH-mediated dimerization of RNF43 or RNF167 with the receptors TβRII or EGFR induces receptor internalization and co-clustering in the perinuclear area. Cells were treated for 5h with 100 nM of the bi-VHH before fixation. E3 ligases were visualized by Myc staining and the receptors by Flag staining. Confocal images of HEK293T cells transfected with (A) E6-Flag-TβRII and Alpha-Myc-RNF43, (B) E6-Flag-TβRII and Alpha-Myc-RNF167, (C) E6-Flag-EGFR and Alpha-Myc-RNF43 and (D) E6-Flag-EGFR and Alpha-Myc-RNF167. Arrows indicate co-clustering of the E3 ligases and receptors in the perinuclear area.

FIG. 6. Bi-functional VHH treatment promotes membrane-bound E3 ligase-mediated internalization of transmembrane receptors from the cell surface. HEK293T cells were transfected with one of the E3 ligases RNF43, RNF128, RNF130 or RNF167 and with the receptors CTLA-4, FLT-3, PD-1 and PD-L1. Cells were left untreated or were treated overnight with 50 nM of the bi-VHH before fixation. The receptors present at the cell surface were visualized by Flag staining in unpermeabilized cells. Confocal images are shown of HEK293T cells transfected with (A) E6-Flag-CTLA-4 and Alpha-Myc-RNF43 or Alpha-Myc-RNF167, (B) E6-Flag-FLT-3 and Myc-RNF43, Alpha-Myc-RNF128 or Alpha-Myc-RNF167, (C) E6-Flag-PD-1 and Alpha-Myc-RNF43, Alpha-Myc-RNF128, Alpha-Myc-RNF130 or Alpha-Myc-RNF167 and (D) E6-Flag-PD-L1 and Alpha-Myc-RNF43, Alpha-Myc-RNF128, Alpha-Myc-RNF130 or Alpha-Myc-RNF167.

FIG. 7. Bi-functional VHH treatment promotes membrane-bound E3 ligase-mediated internalization of type III multispan protein CMTM6 from the cell surface. (A) Schematic of the Snorkel tag for the detection of surface expression of multispan proteins. (B) HEK293T cells were transfected with one of the E3 ligases RNF43, RNF128, RNF130 or RNF167 and with the multispan receptor CMTM6. Cells were left untreated or were treated overnight with 50 nM of the bi-VHH before fixation. The receptors present at the cell surface were visualized by Flag staining of unpermeabilized cells. Confocal images are shown of HEK293T cells transfected with E6-Flag-Snorkel-CMTM6 and Alpha-Myc-RNF43, Alpha-Myc-RNF128, Alpha-Myc-RNF130 or Alpha-Myc-RNF167.

FIG. 8. Validation effect bi-VHH on E3 ligase-target combinations at the endogenous level. (A) Strategy to generate endogenously tagged proteins. (B) Schematic of the approach for cell surface removal of targets by bi-VHH using endogenously tagged versions of E3 ligase-target combinations.

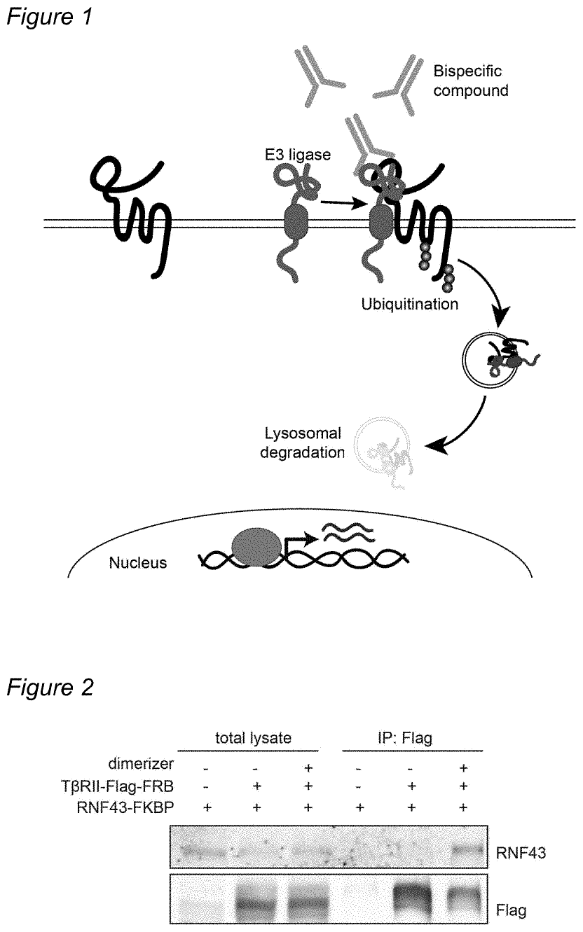

### EXAMPLES

### Example 1

### Materials and Methods

### Cell Culture and Transfection

Human Embryonic Kidney (HEK) 293 T cells were cultured in RPMI (Invitrogen) supplemented with 10% fetal bovine serum (GE Healthcare), 2 mM UltraGlutamine (Lonza), 100 units/mL penicillin and 100 μg/mL streptomycin (Invitrogen). Cells were cultured at 37° C. in 5% CO2. Transfections were performed using FuGENE 6 (Promega) according to the manufacturer's protocol for microscopy, or using PEI for biochemistry. The A/C Heterodimerizer (Takara Bio, #635056) was used at 1 uM overnight at 37° C., control conditions were treated with an equal volume of 100% ethanol. TGFβ was used at 1.5 ng/mL for 45 minutes.

### Constructs and Antibodies

TGF-β type II serine/threonine kinase receptor (TβRII)-Flag-FKBP and -Flag-FRB were provided by Peter ten Dijke (LUMC, Leiden). RNF43-FKBP and —FRB were obtained by inserting the coding sequence for FKBP^{36V} or FRB, respectively, in the C-tail of human RNF43 using Q5 High-Fidelity 2x Master Mix (NEB). All constructs were sequence verified. CD63-GFP was a gift of J. Klumperman (UMCU, Utrecht). The following primary antibodies were used for immunoblotting (IB), immunofluorescence (IF) or immunoprecipitation (IP): rabbit anti-FLAG (Sigma-Aldrich), rat anti-HA (Roche), mouse anti-FLAG (M2; Sigma-Aldrich), mouse anti-Actin (MP Biomedicals) and rabbit anti-RNF43 (Sigma-Aldrich). Primary antibodies were diluted conform manufacturer's instructions. Secondary antibodies used for IB or IF were used 1:8000 or 1:300 respectively and obtained from either Rockland or Invitrogen.

Immunofluorescence and Confocal microscopy. HEK293T cells were grown on glass coverslips coated with laminin (Sigma) in 24-well plates. After overnight transfection cells were fixed in 4% formaldehyde in phosphate buffered saline (PBS). Cells were blocked in buffer containing 2% BSA and 0.1% saponin in PBS for 30 min at room temperature (RT). Subsequently, cells were incubated with primary and secondary antibodies for 1h at RT in blocking buffer. Cells were mounted in Prolong Diamond (Life technologies) and images were acquired with a LSM700 confocal microscopes. Images were analysed and processed with ImageJ.

Immunoprecipitation and western blotting. After transfection, cells were grown to 80% confluency in 10 cm dishes. After washing cells with PBS, cells were scraped and lysed in cell lysis buffer containing 100 mM NaCL, 50 mM Tris pH 7.5, 0.25% Triton X-100, 10% Glycerol, 50 mM NaF, 10 mM Na₃VO₄, 10 μM leupeptin, 10 μM aprotinin and 1 mM PMSF. Lysates were cleared by centrifugation at 16.000×g for 15 min at 4° C. Lysates were taken up in SDS sample buffer and heated for 1 hour at 37° C. For immunoprecipitation, lysates were incubated with 25 μl pre-coupled Flag-M2 beads (Sigma) and incubated overnight at 4° C. After washing, beads were eluted with sample buffer and heated for 1 hour at 37° C. After SDS-PAGE, proteins were transferred via Western blotting onto Immobilon-FL PVDF membranes (Milipore). After blocking with Odyssey blocking buffer (LI-COR), proteins were labelled with the indicated primary antibodies that were detected with goat anti-mouse/rabbit Alexa 680 (Invitrogen), donkey anti-rat Alexa 680 (Invitrogen) or goat anti-mouse/rabbit IRDye 800 (Rockland) using a Amersham Typhoon Biomolecular Imager (GE Health Care).

### Results and Discussion

Forced Dimerization of RNF43 and TGF-β Type II Serine/Threonine Kinase Receptor (TβRII) induces lysosomal localization and degradation of TβRII

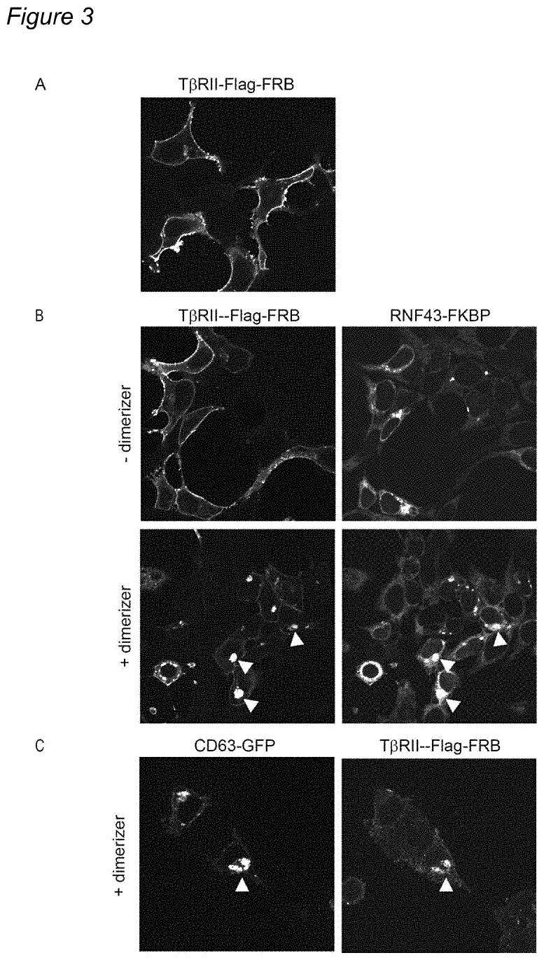

To show proof of concept for redirection of a transmembrane E3 ligase to target a selected cell surface protein for internalization and lysosomal degradation, we used the FKBP/FRB dimerization system. We fused either the FKBP domain or the FRB domain to the C-terminal tails of both TβRII and RNF43. When co-expressed in HEK293T cells, these proteins do not interact (FIG. 2). Upon addition of the A/C dimerizer, however, co-immunoprecipitation of RNF43 and TβRII is induced (FIG. 2). The dimerizer by itself does not interfere with TβRII or RNF43 stability (FIG. 2, whole cell lysate). We next asked whether the forced interaction of RNF43 with TβRII changes the subcellular localization of TβRII. In the absence of dimerizer, TβRII predominantly localizes to the plasma membrane, both in the absence of RNF43 (FIG. 3A) and upon co-expression of RNF43 (FIG. 3B). The addition of the dimerizer, however, strongly induced relocalization of both TβRII and RNF43 to a perinuclear vesicle cluster that is positive for the lysosomal marker CD63 (FIG. 3C). These findings indicate that forced dimerization of RNF43 and TβRII directs increased levels of TβRII to the lysosome.

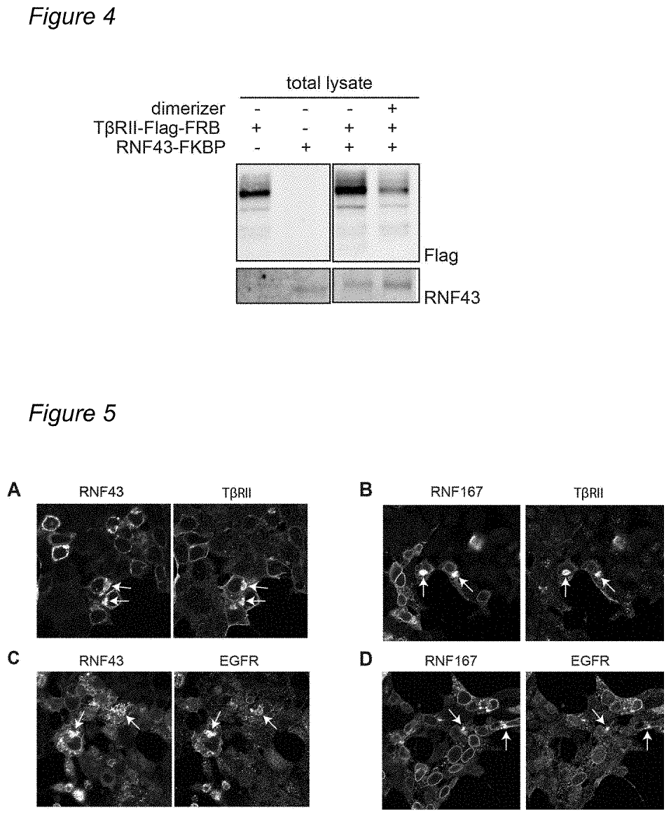

To determine if enhanced lysosomal localization of TβRII results in lower amounts of functional TβRII, we analyzed protein levels using Western Blot. Although RNF43 expression itself has no effect on the stability of TβRII, induced dimerization of RNF43 and TβRII clearly leads to reduced TβRII protein levels (FIG. 4). Together these results indicate that forced dimerization of the transmembrane E3 ligase RNF43 and a normally unrelated transmembrane receptor TβRII targets TβRII for lysosomal degradation.

### Example 2

### Materials and Methods

Cell culture and Transfection

Human Embryonic Kidney (HEK) 293 T cells were cultured in RPMI (Invitrogen) supplemented with 10% fetal bovine serum (GE Healthcare), 2 mM UltraGlutamine (Lonza), 100 units/mL penicillin and 100 μg/mL streptomycin (Invitrogen). Cells were cultured at 37° C. in 5% CO2. Transfections were performed using FuGENE 6 (Promega) according to the manufacturer's protocol.

### Constructs and Antibodies

E6-Flag-TGF-β type II serine/threonine kinase receptor (TβRII) and -Epidermal Growth Factor Receptor (EGFR) and Alpha-myc-RNF43 and RNF167 were obtained by subcloning using Q5 High-Fidelity 2x Master Mix (NEB). All constructs were sequence verified. The following primary antibodies were used for immunofluorescence (IF): rabbit anti-Flag (Sigma-Aldrich) and mouse anti-Myc (hybridoma 9E10). Primary antibodies were diluted conform manufacturer's instructions. Secondary antibodies used for IF were used at 1:300 (Life technologies).

Immunofluorescence and Confocal microscopy. HEK293T cells were grown on glass coverslips coated with laminin (Sigma-Aldrich) in 24-well plates. After overnight transfection cells were incubated 1h with 20 nM Bafilomycin A1 (Sigma-Aldrich) before and during a 5h treatment with 100 nM of the bi-VHHs (VHH Alpha-(G4S)3-VHH E6). After treatment cells were washed two times with warm medium and fixed in 4% formaldehyde in 0.05 M Phosphate buffer pH 7.4. Cells were blocked in buffer containing 2% BSA and 0.1% saponin in PBS for 30 min at room temperature (RD. Subsequently, cells were incubated with primary antibodies against either Flag or Myc for 1 h at RT, followed by the secondary antibodies for 1 h at RT in blocking buffer. Cells were mounted in Prolong Diamond (Life technologies) and images were acquired with an LSM700 confocal microscope. Images were analysed and processed with ImageJ.

### Results and Discussion

### RNF43 and RNF167 Induce Cell Surface Removal of TβRII and EGFR Upon Forced Dimerization Using Bi-Specific VHHs.

To further confirm the functionality of the heterobifunctional molecules of the invention, selected receptors were targeted with E3 ligases by VHH-mediated dimerization of extracellular regions. To this end, we fused epitope tags to the extracellular domains of both the targets (E6 tag) and E3 ligases (Alpha tag). We selected these epitope tags for recognition by VHHs (Gotzke et al., 2019, Nature Communications, 10(1), 1-12; Ling et al., 2019, Molecular Immunology, 114(July), 513-523) and we generated bi-specific VHHs (bi-VHHs) against these two epitopes to allow for VHH-mediated dimerization. To determine alterations in the localization of proteins, we also incorporated a Myc epitope tag for the E3 ligases and a Flag epitope tag for the receptors. When co-expressed in HEK293T cells, none of the receptors colocalized with any of the E3 ligases: E3 ligases mainly localized to intracellular compartments and the receptors mainly at the plasma membrane (data not shown). However, upon 5 h treatment with bi-VHH both RNF43 and RNF167 induced removal of TβRII as well as EGFR from the plasma membrane. Moreover, internalized proteins co-clustered in the perinuclear area in bafilomycin-treated cells, which inhibits lysosomal turnover, indicating accumulation of E3 ligases and their targets in late endosomal/lysosomal structures (FIG. 5A-D). These findings show that heterobifunctional molecules, such as bi-VHHs, can be used to deliberately dimerize a transmembrane E3 ligase with a selected transmembrane receptor, thereby inducing receptor removal from the cell surface.

### Example 3

### Materials and Methods

### Cell Culture and Transfection

Human Embryonic Kidney (HEK) 293 T cells were cultured in RPMI (Invitrogen) supplemented with 10% fetal bovine serum (GE Healthcare), 2 mM UltraGlutamine (Lonza), 100 units/mL penicillin and 100 μg/mL streptomycin (Invitrogen). Cells were cultured at 37° C. in 5% CO2. Transfections were performed using FuGENE 6 (Promega) or Effectene (Qiagen) according to the manufacturer's protocol.

### Constructs and Antibodies

E6-Flag-Cytotoxic T-lymphocyte-associated antigen 4 (CTLA-4), Receptor-type tyrosine-protein kinase FLT3 (FLT-3), Programmed cell death protein 1 (PD-1) and Programmed cell death 1 ligand 1 (PD-L1) and Alpha-myc-RNF43, RNF128, RNF130 and RNF167 were obtained by subcloning using Q5 High-Fidelity 2x Master Mix (NEB). All constructs were sequence verified. The following primary antibodies were used for immunofluorescence (IF): rabbit anti-Flag or mouse anti-Flag (Sigma-Aldrich). Primary antibodies were diluted conform manufacturer's instructions. Secondary antibodies used for IF were used at 1:300 (Life technologies).

Immunofluorescence and Confocal microscopy. HEK293T cells were grown on glass coverslips coated with laminin (Sigma-Aldrich) in 24-well plates. Six hours after transfection cells were incubated overnight with 50 nM of the bi-VHHs (VHH Alpha-(G4S)3-VHH E6). After treatment cells were washed two times with warm medium and fixed in 4% formaldehyde in 0.05 M Phosphate buffer pH 7.4. Cells were blocked in buffer containing 2% BSA in PBS for 30 min at room temperature (RT). Subsequently, cells were incubated with primary antibody against Flag for 1 h at RT, followed by the secondary antibody for 1h at RT in blocking buffer. Cells were mounted in Prolong Diamond (Life technologies) and images were acquired with an LSM700 confocal microscope using a 5× objective lens or with an EVOS-M5000 microscope using a 20× objective lens. Images were analyzed and processed with ImageJ.

### Results and Discussion

### Specific E3 Ligase-Target Combinations Allow for Surface Removal of a Target Upon Forced Dimerization Using Bi-Specific VHHs.

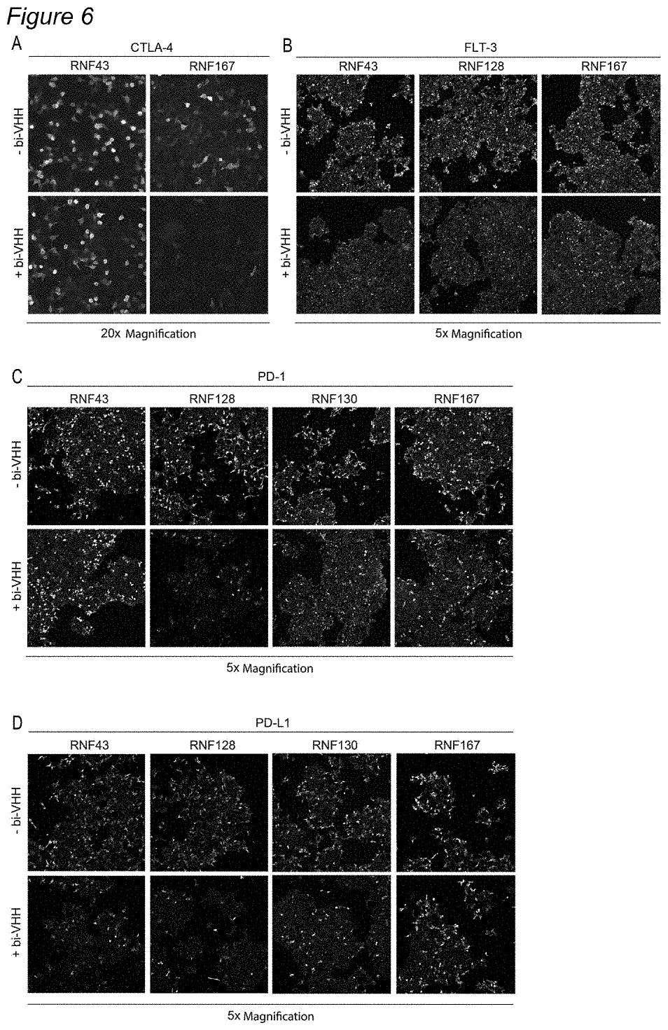

To screen for additional candidate E3 ligase-receptor combinations, we generated the following constructs Alpha-Myc-RNF128, Alpha-Myc-RNF130, E6-Flag-CTLA-4, E6-Flag-FLT-3, E6-Flag-PD-1 and E6-Flag-PD-L1 in addition to the previously generated constructs. When co-expressed in HEK293T cells, CTLA-4, FLT-3, PD-1 and PD-L1 all localized to the cell surface. Upon overnight treatment with bi-VHHs, the following E3-target combinations lead to target removal of the surface: CTLA-4 and RNF167; FLT-3 and RNF43, RNF128 or RNF167, PD-1 and RNF128, RNF130 or RNF167 and PD-L1 and RNF43, RNF128 or RNF130 (FIG. 6A-D). These findings expand the range of use for heterobifunctional molecules, such as bi-VHHs, to deliberately dimerize various transmembrane E3 ligases with a selection of transmembrane receptors, thereby inducing removal of these receptors from the cell surface. These findings also underline that not all combinations are effective.

### Example 4

### Materials and Methods

### Cell Culture and Transfection

Human Embryonic Kidney (HEK) 293 T cells were cultured in RPMI (Invitrogen) supplemented with 10% fetal bovine serum (GE Healthcare), 2 mM UltraGlutamine (Lonza), 100 units/mL penicillin and 100 μg/mL streptomycin (Invitrogen). Cells were cultured at 37° C. in 5% CO2. Transfections were performed using FuGENE 6 (Promega) or Effectene (Qiagen) according to the manufacturer's protocol.

### Constructs and Antibodies

E6-Flag-Snorkel CKLF-like MARVEL transmembrane domain-containing family protein 6 (CMTM6) was obtained by subcloning using Q5 High-Fidelity 2x Master Mix (NEB). The construct was sequence verified. Mouse anti-Flag (Sigma-Aldrich) primary antibody was used for immunofluorescence (IF). Primary antibody was diluted conform manufacturer's instructions. Secondary antibody used for IF were used at 1:300 (Life technologies).

Immunofluorescence and Confocal microscopy.

HEK293T cells were grown on glass coverslips coated with laminin (Sigma-Aldrich) in 24-well plates. Six hours after transfection cells were incubated overnight with 50 nM of the bi-VHHs (VHH Alpha-(GGGGS)3-VHH E6). After treatment cells were washed two times with warm medium and fixed in 4% formaldehyde in 0.05 M Phosphate buffer pH 7.4. Cells were blocked in buffer containing 2% BSA in PBS for 30 min at room temperature (RT). Subsequently, cells were incubated with primary antibody against Flag for 1 h at RT, followed by the secondary antibody for 1 h at RT in blocking buffer. Cells were mounted in Prolong Diamond (Life technologies) and images were acquired with an LSM700 confocal microscope using a 5× objective. Images were analyzed and processed with ImageJ.

### Results and Discussion

### RNF43 and RNF128 Induce Cell Surface Removal of the Multispan Receptor CMTM6 Upon Forced Dimerization Using Bi-Specific VHHs.

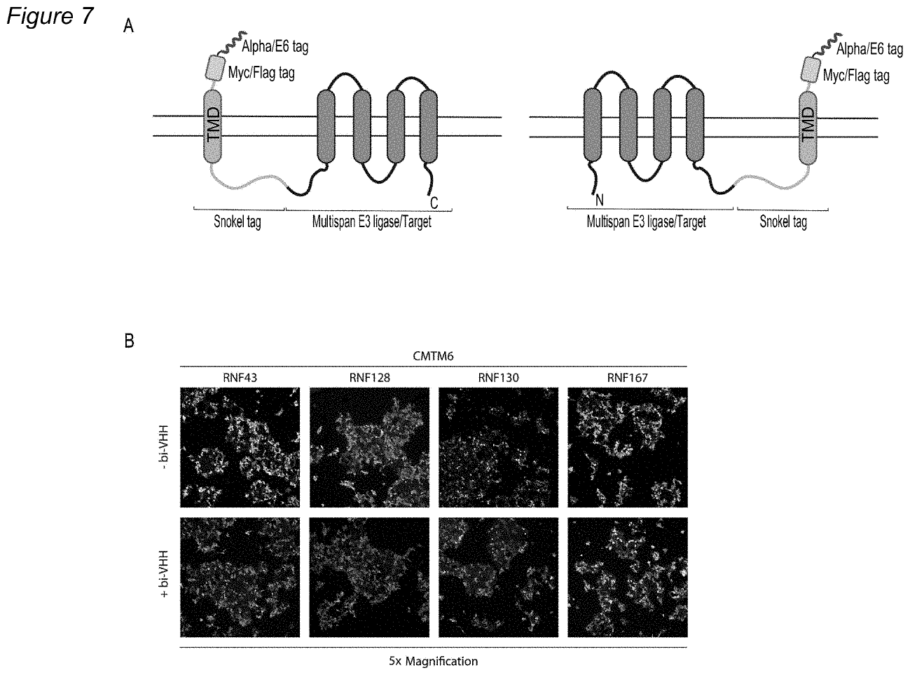

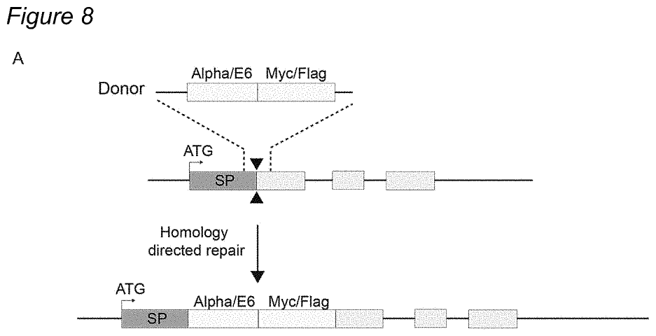

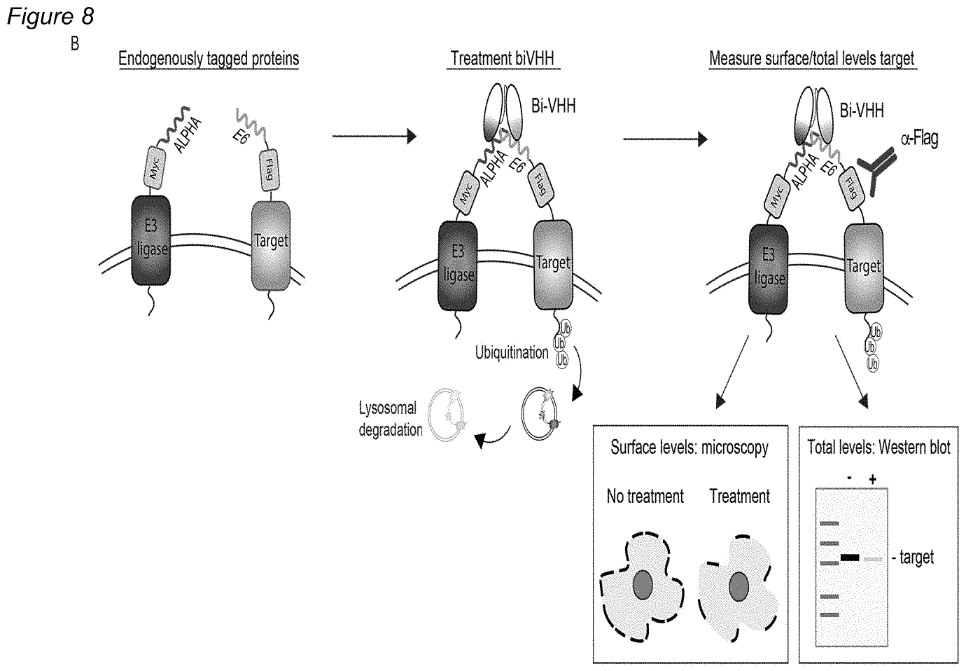

To expand the applicability of the heterobifunctional molecules of the invention to type-2 or type 3 transmembrane proteins, we made use of the Snorkel tag. Type 2 and 3 transmembrane proteins have their N-terminus or both their N- and C-terminus located intracellularly respectively, In order to detect their surface expression a Snorkel tag can be added to the intracellular N-or C-terminal region, allowing the tagging and the extracellular detection of the proteins without perturbing their structure or sub-cellular localization. The Snorkel tag is composed of a transmembrane domain (TMD) flanked by a linker region and two epitope tags. Depending on the multispan protein of interested, we incorporated the Alpha-Myc tags for multispan E3 ligases or the E6-Flag tags for multispan targets (FIG. 7A). As an example, we generated E6-Flag-Snorkel-CMTM6 in addition to the previously generated constructs. When co-expressed in HEK293T cells, CMTM6 localized to the cell surface. Upon overnight treatment with bi-VHHs, RNF43 and to a lesser extend RNF128 were able to remove CMTM6 from the surface upon treatment with the biVHH (FIG. 7 B).

These findings expand the range of use for heterobifunctional molecules, such as bi-VHHs, to deliberately dimerize various transmembrane E3 ligases with multispan receptors such as CMTM6, thereby inducing removal of these receptors from the cell surface. In addition, these findings again underline the need for screening effective E3 ligase-target combinations as not all combinations are effective.

### Example 5

To validate the promising combinations emerging from the above screening in a physiological setting, we will use CRISPR/Cas9 technology to generate (cancer) cell lines expressing endogenously tagged E3 ligases and targets. We will use guide RNAs in combination with donor DNA for the Alpha-Myc or E6-Flag tags that will facilitate the insertion of these tags between the signal peptide (SP) and the coding sequence for the first mature amino acid in the endogenous locus of the E3 ligase or the target (FIG. 7A). Using these cell lines, we will assess the removal of the endogenous target from the cell surface upon forced dimerization with the bi-VHH by either microscopy, FACS or Western blotting (FIG. 7B).

1. A method for identifying an effective combination of a transmembrane E3 ubiquitin ligase and a membrane-bound protein, wherein the combination is effective when the transmembrane E3 ubiquitin ligase is capable of decreasing the surface level of the membrane-bound protein upon simultaneous binding to a heterobifunctional molecule, preferably by ubiquitination of the membrane-bound protein, and wherein the method comprises the steps of:
a) Providing a cell, wherein the cell expresses the transmembrane E3 ubiquitin ligase and the membrane-bound protein at its cell surface;
b) Exposing the cell to the heterobifunctional molecule, wherein the heterobifunctional molecule comprises:
i) a first binding domain capable of specific binding to an extracellular portion of the transmembrane E3 ubiquitin ligase; and
ii) a second binding domain capable of specific binding to an extracellular portion of the membrane-bound protein; and
c) determining the surface level of the membrane-bound protein of the cell,
wherein a decrease in the surface level of the membrane-bound protein indicates that the combination is an effective combination, and wherein the decrease is preferably a decrease as compared to the surface level of the membrane-bound protein of the cell prior to step b).

2. The method according to claim 1, wherein the membrane-bound protein is a transmembrane protein.

3. The method according to claim 1, wherein the transmembrane E3 ubiquitin ligase is selected from the group consisting RNF43, RNF167, ZNRF3, RNF13, AMFR, MARCH1, MARCH2, MARCH4, MARCH8, MARCH9, RNF149, RNF145, RNFT1, RNF130 and RNF128.

4. The method according to claim 1, wherein at least one of:
the transmembrane E3 ubiquitin ligase comprises a first extracellular non-native epitope tag, and wherein the first binding domain of the heterobifunctional molecule binds to the first non-native epitope tag; and
the membrane-bound protein comprises a second extracellular non-native epitope tag, and wherein the second binding domain of the heterobifunctional molecule binds to the second non-native epitope tag.

5. The method according to claim 4, wherein the first and second non-native epitope tags are different tags.

6. The method according to claim 4, wherein the first non-native epitope tag is at least one of an alpha tag and an E6 tag, and/or wherein the second non-native epitope tag is at least one of an alpha tag and an E6 tag.

7. The method according to claim 4, wherein at least one of the first and second non-native epitope tag is located in at least one of
i) the N-terminus;
ii) the C-terminus; and/or
iii) an extracellular loop region,
of respectively the transmembrane E3 ubiquitin ligase and the membrane-bound protein.

8. The method according to claim 1, wherein the heterobifunctional molecule is a bi-specific antibody, preferably a bi-specific nanobody.

9. The method according to claim 8, wherein the first binding domain of the heterobifunctional molecule is an anti-Alpha VHH and the second binding domain is an anti-E6 VHH, or wherein the first binding domain of the heterobifunctional molecule is an anti-E6 VHH and the second binding domain is an anti-Alpha VHH.

10. The method according to claim 1, wherein the membrane-bound protein comprises a third non-native epitope tag and/or wherein the transmembrane ubiquitin E3 ligase comprises a fourth non-native epitope tag, preferably wherein the third and/or fourth epitope tag is at least one of a His-tag, FLAG-tag, and a myc-tag.

11. The method according to claim 1, wherein the cell surface levels of the membrane-bound protein in step c) are determined by detecting the protein on the cell surface, preferably by immunofluorescence.

12. The method according to claim 1, wherein the combination is effective when the cell surface levels of the membrane-bound protein are decreased at least about 10%, 20%, 30%, 40%, 50%, 60%, 70%, 80%, 90% or at least about 95% as compared to the cell surface levels of the membrane-bound protein prior to step b), preferably at least about 60%, 70%, 80%, 90% or at least about 95% as compared to the cell surface levels of the membrane-bound protein prior to step b).

13. The method according to claim 4, wherein in step a) a first and a second cell is provided, wherein
the first cell expresses a first transmembrane E3 ubiquitin ligase and a first membrane-bound protein at its cell surface; and
the second cell expresses a second transmembrane E3 ubiquitin ligase and the first membrane-bound protein and its cell surface,
wherein the first and second transmembrane E3 ubiquitin ligase are different ligases comprising the same first extracellular non-native epitope tag;
wherein in step b) the first and the second cell is exposed the heterobifunctional molecule,
wherein the heterobifunctional molecule comprises:
i) a first binding domain capable of specific binding to the first extracellular non-native epitope tag; and
ii) a second binding domain capable of specific binding to an extracellular portion of the membrane-bound protein, preferably to the second non-native epitope tag; and
wherein in step c) the surface level of the membrane-bound protein of the first and second cell are determined, and wherein a combination is effective when the cell surface levels of the membrane-bound protein in the first cell are decreased at least about 5%, 10%, 20%, 30%, 40%, 15 50%, 60%, 70%, 80%, 90% or at least about 95% as compared to the cell surface levels of the membrane-bound protein in the second cell after step b).

14. The method according to claim 13, wherein a third, fourth or further cells are provided expressing respectively a third, a fourth or a further transmembrane E3 ubiquitin ligase and the first membrane-bound protein at their cell surface,
wherein the transmembrane E3 ubiquitin ligases are different ligases comprising the same first extracellular non-native epitope tag,
and wherein the combination is effective when the cell surface levels of the membrane-bound protein in the first cell are decreased at least about 5%, 10%, 20%, 30%, 40%, 50%, 60%, 70%, 80%, 90% or at least about 95% as compared to the cell surface levels of the membrane-bound protein in the second, third, fourth and further cells after step b),
and wherein preferably the method is performed in a multiplexed manner.

15. The method according to claim 1, wherein the decrease in the surface level of the membrane-bound protein is determined by a decrease in the total amount of the membrane-bound protein in the cell, preferably as determined by microscopy, biochemical analysis and/or FACS.

16. The method according to claim 1, wherein the cell provided in step a) overexpresses, optionally permanently overexpresses, at least one of the transmembrane E3 ubiquitin ligase and the membrane-bound protein.

17. The method according to claim 1, wherein the cell provided in step a) expresses the transmembrane E3 ubiquitin ligase and the membrane-bound protein at endogenous levels.

18. The method according to claim 17, wherein in the cell provided in step a) a genomic sequence encoding the transmembrane E3 ubiquitin ligase has been modified to incorporate a sequence encoding the first, and optional fourth, non-native epitope tag.

19. The method according to claim 17, wherein in the cell provided in step a) a genomic sequence encoding the membrane-bound protein has been modified to incorporate a sequence encoding the second, and optional third, non-native epitope tag.

20. The method according to claim 1, wherein the heterobifunctional molecule comprises a peptide linker between the first binding domain and the second binding domain, and wherein preferably the peptide linker is (GGGGS)n, wherein n is preferably 1, 2, 3, 4, 5, 6, 7, 8, 9, 10, 11, 12, 13, 14 or 15, preferably wherein n is 3 or 5.

21. A method for producing a heterobifunctional molecule comprising a first and a second binding domain, wherein
i) the first binding domain is capable of specific binding to an extracellar portion of a transmembrane E3 ubiquitin ligase; and
ii) the second binding domain is capable of specific binding to an extracellular portion of a transmembrane protein, and wherein the transmembrane E3 ligase and the transmembrane protein are an effective
combination as determined in the method of claim 1.

22.-25. (canceled)
